(19) United States
(12) Patent Application Publication
(10) Pub. No.: US 2013/0059783 A1
(43) Pub. Date: Mar. 7, 2013

(54) COMPOSITIONS AND METHODS FOR EXPANDING BFU-E CELLS

(71) Applicants: Johan Flygare, Lund (SE); Harvey F. Lodish, Brookline, MA (US)

(72) Inventors: Johan Flygare, Lund (SE); Harvey F. Lodish, Brookline, MA (US)

(21) Appl. No.: 13/634,399

(22) PCT Filed: Mar. 12, 2011
(86) PCT No.: PCT/US11/28248

§ 371 Date: Nov. 20, 2012

Related U.S. Application Data
(60) Provisional application No. 61/313,623, filed on Mar. 12, 2010.

(51) Int. Cl. C12N 5/078; A61K 38/02; A61P 7/06; C12Q 1/02

(52) U.S. Cl. 514/13.5; 435/372; 435/29

(57) ABSTRACT

The invention provides methods of expanding BFU-E cells comprising contacting one or more BFU-E cells with a hypoxia inducible factor 1 activator and a glucocorticoid receptor (GR) activator, e.g., a GR agonist. In some embodiments, the FIIF-1 activator is a prolyl hydroxylase inhibitor (PHI). In some embodiments, the method comprises culturing BFU-E in medium containing a HIF-1a activator and a glucocorticoid receptor (GR) activator, e.g., a GR agonist. In some embodiments the BFU-E cells are human cells. The invention provides cell culture media useful for expanding BFU-Es, wherein the cell culture media comprise a HIF-1 activator and a GR activator (e.g., a GR agonist) The invention provides a method comprises administering a HIF-1 activator and a GR agonist to a subject in need thereof. In some embodiments, the subject suffers from anemia. In some embodiments, the anemia is an Epo-resistant anemia. In some embodiments, the anemia is Diamond-Blackfan anemia.

### RELATED APPLICATIONS

This application claims the benefit of, and priority to, U.S. provisional application Ser. No. 61/313,623, filed Mar. 12, 2010, the entire content of which is incorporated by reference herein.

### GOVERNMENT FUNDING

This invention was made in part with government support from National Institutes of Health grant NIH grant P01 HL 32262. The government has certain rights in the invention.

### BACKGROUND OF THE INVENTION

Blood cells are normally produced continuously from a relatively small number of hematopoietic stem cells (HSCs) that have the ability to both self-renew and to generate progenitor cells that subsequently give rise to all mature blood cell types. The differentiation pathways leading to formation of mature blood cells are regulated in part by specific growth factors binding to their receptors. Multi-potential and lineage-restricted progenitors can be identified based on the morphological characteristics of discrete cell clusters or colonies to which they give rise during in vitro culture in suitable materials such as methylccllulose in the presence of various growth factor(s).

Red blood cells (also referred to as RBCs or erythrocytes) are the most abundant blood cell type. They are found in virtually all vertebrates and fulfill the essential function of transporting oxygen (O2) from the lungs (or gills) to other body tissues via the circulatory system. The RBC cytoplasm contains large amounts of hemoglobin, an iron-containing biomolecule that binds O2 during RBC transit through pulmonary capillaries and releases it under conditions of lower O2 partial pressure that prevail in capillaries elsewhere in the body.

The process by which RBCs are produced (erythropoiesis) has been intensively studied. HSCs give rise to multipotent common myeloid progenitor cells, which in turn give rise to more developmentally restricted progenitors, leading to progenitor cells that are committed to the erythroid lineage. Erythropoietin (Epo) is a 34 kDa glycoprotein growth factor that plays a crucial role in promoting the survival, proliferation, and differentiation of mammalian erythroid progenitor cells. The BFU-E (burst-forming unit erythroid) is the earliest committed red blood cell progenitor and responds to growth factors such as SCF, IL-3, and Epo. CFU-E (colony-forming unit erythroid) are committed erythroid progenitor cells that specifically respond to Epo.

Anemia associated with chronic renal insufficiency or malignancy can often be successfully treated with recombinant erythropoietin (Epo). Epo promotes survival, proliferation, and terminal differentiation of CFU-E cells and more mature erythroblasts. Since normal Epo levels are very low, red cell output from CFU-E cells can often be increased more than one order of magnitude by injection of recombinant Epo. However because each CFU-E cell can typically undergo only a few (e.g., 3-5) cell divisions under maximum Epo-stimulation, the number of CFU-E cells can eventually limit the response to Epo. Furthermore, anemias associated with a number of other conditions do not respond well (if at all) to Epo. There is a need in the art for drugs that could allow improved treatment of anemia, e.g., anemias that cannot be appropriately treated with Epo.

### SUMMARY OF THE INVENTION

The invention provides a method of expanding BFU-E cells comprising contacting one or more BFU-E cells with a hypoxia inducible factor 1 activator and a glucocorticoid receptor (GR) activator, e.g., a GR agonist. In some embodiments, the HIF-1 activator is a prolyl hydroxylase inhibitor (PHI). In some embodiments, the method comprises culturing BFU-E in medium containing a HIF-1a activator and a glucocorticoid receptor (GR) activator, e.g., a GR agonist. In some embodiments the BFU-E cells are human cells. In some embodiments the method comprises administering a HIF-1 activator and a glucocorticoid receptor (GR) activator, e.g., a GR agonist, to a subject, e.g., an individual suffering from or at risk of anemia. In some embodiments the subject is human.

In another aspect, the invention provides a method of expanding BFU-E cells in vitro comprising culturing or more BFU-E cells in media comprising a HIF-1 activator, a GR activator (e.g., a GR agonist), or both. In some embodiments, BFU-E cells are obtained from a starting population comprising fetal liver cells, fetal spleen cells, peripheral blood cells, bone marrow cells, or umbilical cord blood cells. In some embodiments, BFU-Es are obtained by in vitro culture and differentiation of hematopoietic stem cells (HSCs). In some embodiments, BFU-Es are obtained from human CD34+ cells. In some embodiments, the method results in an increased number of CFU-Es. In some embodiments, the method results in an increased number of mature, functional RBCs. In some embodiments, the media contains both a HIF-1 activator and a GR agonist. In some embodiments, the method further comprises administering expanded cells to a subject in need thereof.

In another aspect, the invention provides a method of treating a subject suffering from or at risk of anemia, the method comprising administering a HIF-1 activator and a GR activator, e.g., a GR agonist, to the subject. In some embodiments, the anemia is anemia of chronic disease, anemia associated with chemotherapy, anemia associated with renal disease, anemia associated with infection, anemia associated with aging, or anemia associated with blood loss. In some embodiments, the subject is at risk of anemia, e.g., because the subject is expected to undergo surgery within 4 weeks following administration of the HIF-1 activator and the GR activator (e.g., GR agonist). In some embodiments, the anemia is due to bone marrow failure. In some embodiments, the anemia is Diamond-Blackfan syndrome. In some embodiments, the subject has experienced acute or chronic blood loss. In some embodiments, the anemia results at least in part from hemolysis.

In another aspect, the invention provides a method of treating a subject suffering from or at risk of an Epo-resistant anemia comprising administering a HIF-1 activator to the subject. In some embodiments, the method comprises administering a GR activator, e.g., a GR agonist, in combination with the HIF-1 activator.

In some embodiments, the Epo-resistant anemia is anemia of chronic disease, anemia associated with chemotherapy, anemia associated with renal disease, or anemia associated with infection, or anemia due to bone marrow failure. In some embodiments, the Epo-resistant anemia is Diamond-Blackfan syndrome.

In another aspect, the invention provides a method of treating a subject suffering from an Epo-resistant anemia, wherein the Epo-resistant anemia is an anemia that is not currently treated with a GR agonist, the method comprising administering to the subject a compound that enhances survival or self-renewal of BFU-E cells. In some embodiments, the compound that enhances survival or self-renewal of BFU-E cells is a HIF-1 activator. In some embodiments, the compound that enhances survival or self-renewal of BFU-E cells is GR agonist. In some embodiments, the method comprises administering a HIF-1 activator and a GR agonist to the subject.

In another aspect, the invention provides a composition comprising a HIF-1 activator and a GR activator, e.g., a GR agonist. In some embodiments, the HIF-1 activator is a PHI. In some embodiments, the GR agonist is a glucocorticoid, e.g., prednisone. In some embodiments, the glucocorticoid is a synthetic glucocorticoid or other GR ligand. In some embodiments, the composition is a cell culture medium. In some embodiments, the composition is a pharmaceutical composition which, in some embodiments, is suitable for oral administration.

In some aspects, the invention provides a cell culture medium comprising a HIF-1a activator and a GR activator, e.g., a GR agonist. In some embodiments, the HIF-1a activator is a PHI. In some embodiments, the HIF-1a activator is DMOG. In some embodiments, the GR agonist is a glucocorticoid, e.g., dexamethasone. In some embodiments, the HIF-1a activator is a prolyl hydroxylase inhibitor and the GR agonist is a glucocorticoid. In some embodiments, the cell culture medium comprises Epo, SCF, and/or IGF-1. In some embodiments, the medium comprises Epo and SCF. In some embodiments, the medium comprises Epo, SCF, and IGF-1. In some embodiments, the medium is serum-free.

The invention further provides a container containing a cell culture medium of the invention, wherein said container is suitable for expanding cells. In some embodiments, the container further contains erythroid progenitor cells. In some embodiments, the cells comprise human erythroid progenitor cells. In some embodiments, the cells comprise human CD34+ cells.

In another aspect, the invention provides a purified cell population, wherein at least 75% of the cells are BFU-E cells. In some embodiments, the purified cell population contains no more than 2% CFU-G/M/Mk cells and/or no more than 1% GEMM cells.

In another aspect, the invention provides a purified cell population, wherein at least 50% of the cells are CFU-E cells.

In another aspect, the invention provides a method of purifying BFU-E cells from a population of cells that comprises one or more BFU-E cells, the method comprising steps of: (a) depleting the population of cells that are positive for Ter119, CD16, CD32, Sca-1, and/or CD41; and (b) selecting cells that are (i) c-kit positive and (ii) CD71^{10% low} or CD24a^{10%} low. In some embodiments, step (b) comprises selecting cells that are CD71^{10% low} and CD24a^{10% low}. In another aspect, the invention provides a method of purifying CFU-E cells from a population of cells that comprises one or more CFU-E cells, the method comprising steps of: (a) depleting the population of cells that are positive for Ter119, CD16, CD32, Sca-1, and/or CD41; and (b) selecting cells that are (i) c-kit positive and (ii) CD71^{20% high}. In some embodiments of the afore-mentioned purification methods, the population of cells are selected from the group consisting of bone marrow cells, umbilical cord blood cells, peripheral blood cells, and fetal liver cells.

In another aspect, the invention provides a method for determining whether a compound promotes expansion of BFU-E cells comprising (a) providing a purified cell population comprising at least 80% BFU-E cells; (b) contacting the purified cell population of (a) with a test compound; (c) assessing the extent to which the cell population increases during a subsequent culture period, wherein if the cell population increases to a greater extent than would be expected had the cell population not been contacted with the compound, then the compound promotes expansion of BFU-E cells. The method can comprise assessing the number of BFU-E-derived colonies cells that develop from at least some of the cells contacted with the compound. In some embodiments, the compound is a GR agonist.

In certain embodiments of aspects of the invention that relate to a HIF-1 activator, the HIF-1 activator is a prolyl hydroxylase inhibitor (PHI). In some embodiments, the PHI specifically inhibits one or more HIF-1 prolyl hydroxylases. In some embodiments of aspects of the invention that relate to GR activators, the GR activator is a GR agonist, e.g., a glucocorticoid, e.g., prednisone. In some embodiments, the GR agonist is a synthetic glucocorticoid.

In one aspect, the invention provides kits useful for expanding BFU-Es. The kit can comprise components of any of the inventive media described herein, and optionally including instructions for expanding BFU-Es in vitro. In some embodiments, a kit comprises a GR activator, a HIF-1a activator, and, optionally, SCF, IGF-1, and/or Epo (e.g., any 1, 2, or 3 of SCF, IGF-1, and Epo). For example, in some embodiments, a kit comprises a GR activator, a HIF-1a activator, SCF, IGF-1, and Epo. In some embodiments, components are provided as individual aliquots suitable for addition to a culture medium. In some embodiments, components are provided as a “cocktail” comprising two or more components suitable for addition to a culture medium. In some embodiments, a base culture medium is provided as part of a kit or separately.

The practice of the present invention will typically employ, unless otherwise indicated, conventional techniques of molecular biology, cell culture, recombinant nucleic acid (e.g., DNA) technology, immunology, nucleic acid and polypeptide synthesis, detection, manipulation, and quantification, and RNA interference that are within the skill of the art. See, e.g., Ausubel, F., et al., (eds.), Current Protocols in Molecular Biology, Current Protocols in Immunology, Current Protocols in Protein Science, and Current Protocols in Cell Biology, all John Wiley & Sons, N.Y., editions as of December 2008; Sambrook, Russell, and Sambrook, Molecular Cloning: A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988. Other useful references include Abraham, D J (ed.), Burger's Medicinal Chemistry and Drug Discovery, 6^{th} ed. Wiley, 2003; Goodman and Gilman's The Pharmacological Basis of Therapeutics, 11th Ed., McGraw Hill, 2006, Katzung, B. (ed.) Basic and Clinical Pharmacology, McGraw-Hill/Appleton & Lange; 11th edition (July 2009); Fauci, et al. (eds.) Harrison's principles of internal medicine, 17^{th} ed. New York: McGraw-Hill, 2008; Lichtman, M, et al. Williams Hematology, 7^{th} edition, McGraw-Hill, 2005; Kronenberg, H., et al. (eds.), Williams Textbook of Endocrinology, Saunders; 11^{th} ed. 2007)). All patents, patent applications, publications, references, web sites, databases, etc., cited in the instant patent application are incorporated by reference in their entirety. In the event of a conflict or inconsistency with the specification, the specification shall control. The Applicants reserve the right to amend the specification based on any of the incorporated references. None of the content of the incorporated references shall limit the invention.

### BRIEF DESCRIPTION OF THE DRAWINGS

FIGS. 1A-1F. Glucocorticoids stimulate stress erythropoiesis by enhancing erythroblast output predominantly from BFU-E progenitors. We determined if addition of 100 nM Dex changes the maximum number of erythroblasts that sorted CFU-E and BFU-E cells produce in serum-free liquid erythroid expansion medium (SFELE) with or without 100 nM Dex. (A) Production of erythroblasts from sorted CFU-E (CD71^{20% high}) cells peaks at day 3 with 28 and 15-fold expansion with and without Dex respectively (n=4). Error bars show one standard deviation. (B) BFU-E (CD71^{10% low}) cells cultured with 100 nM Dex expand 8,800-fold with a peak at day 8, and expand 650-fold (9 cell divisions) without Dex with the peak at day 5 (n=5). Error bars show one standard deviation. (C) Sorted BFU-E (CD71^{10% low}) cells were cultured in SFELE medium with different concentrations of Dex. Shown is the fraction of c-kit+ cells after 72 hours. (D) A second Dex dose-response curve was generated based on the total number of BFU-E daughter cell divisions at day 6. (E) 180 single BFU-E cells were sorted into individual wells of 96 well plates containing SFELE medium with 100 nM Dex and 180 into medium without Dex. Cell numbers in each clone were estimated under the microscope on Day 8. 80% of sorted BFU-E cells form more than 100 daughter cells in erythroid expansion culture. (F) To determine how Dex affects early-BFU-E cell proliferation, cells from 53 BFU-E clones (50 cultured with Dex and 3 without Dex) that were scored as 1000+ at day 8 were counted using a hemocytometer. While individual BFU-E cells were able to generate more than 300,000 erythroblasts in presence of 100 nM Dex, the most productive BFU-E cell without Dex generated 4,100 erythroblasts.

FIGS. 2A-2B. The effect of glucocorticoids on miRNA and mRNA expression in BFU-E cells determined by next generation sequencing. Sorted CD71^{10% low} BFU-E cells were cultured as detailed in FIG. 1 for 4 hours either in the absence or presence of 100 nM Dex, and RNA extracted. Both mRNAs and miRNAs were subjected to Illumina deep sequencing. (A) The miRNA expression R-I plot shows on the Y axis the log 2 ratio of expression of individual miRNAs in Dex-stimulated versus nonstimulated cells. Positive values mean higher expression in BFU-E cells treated with 100 nM Dex. The X axis shows the average expression of individual miRNAs, plotted as the log 2 of the product of expression of the miRNA in stimulated and nonstimulated cells. (B) A plot similar to that in A for expression of individual mRNAs. mRNA expression was normalized using the RPKM method, which determines the relative expression of each gene to all expressed genes by giving each gene an RPKM count (number of uniquely aligning reads per kb exon per million total reads).

FIGS. 3A-3B. DMOG synergizes with Dexamethasone to increase the number of erythroblasts formed from a BFU-E cell 300-fold. The experimental protocol was similar to that used in FIGS. 1A and B except that the CFU-E and BFU-E cells were purer (CD71 and CD24a^{20% high} and CD71 and CD24a^{10% low}, respectively). Cultures contained or not 100 nM Dex and/or 333 μM DMOG, as indicated. (A) Proliferation of sorted CFU-E cells. Cells do not significantly increase proliferation in response to Dex or DMOG alone, while addition of both Dex and DMOG increases proliferation less than 2-fold (n=4). Error bars show one standard deviation. (B) Proliferation of sorted BFU-E cells. Cells increase proliferation 2-fold with DMOG, 42-fold with Dex and 306-fold with both Dex and DMOG. The synergistic effect of Dex and DMOG is shown by the fact that DMOG increases the stimulatory effect of Dex on BFU-E proliferation 7.3-fold (n=4). Error bars show one standard deviation.

FIG. 4. DMOG stimulation of BFU-E cell proliferation is greatly enhanced by the presence of glucocorticoids. As in FIG. 3, cultures of CD71^{10% low} BFU-E cells were established in SFELE medium containing 0 nM, 1 nM, 10 nM, or 100 nM Dex with different concentrations of DMOG dissolved in water. Cells were counted from day 4 until the day cell counts dropped. Without Dex 333 μM DMOG had relatively modest effect on BFU-E proliferation, while addition of 1 nM Dex allows 333 μM DMOG to enhance BFU-E proliferation 12-fold as compared with DMOG alone (n=4).

FIGS. 5A-5C. Proliferation of erythroid progenitor in the Lin-Sca-1-c-kit+ bone marrow progenitor population is synergistically enhanced by DMOG and Dex. (A) Lin^{neg}, c-kit^{pos}, Sca-1^{neg} bone marrow cells were cultured in SFELE medium with no additions (control), 333 μM DMOG, 1 nM or 100 nM Dex, 333 μM DMOG plus 1 nM Dex, or 333 μM DMOG plus 100 nM Dex. Total cell number was counted daily and normalized to the number of cells added to the culture. (n=4). Error bars show one standard deviation. (B) and (C) May-Grünewald Giemsa staining of bone marrow Lin^{neg}, c-kit^{pos}, Sca-1^{neg} cells after 11 days of culture in medium with 333 μM DMOG plus 100 nM Dex (B), and fetal liver BFU-E cells after 10 days culture in the same medium (C). In both cultures only erythroid cells are present.

FIG. 6. Erythroblast formation from of human peripheral blood erythroid progenitors is synergistically enhanced by DMOG and Dex. Human mobilized CD34+ cells were first thawed and cultured in serum free medium supplemented with StemSpan® CC100 cytokine mix containing rhSCF, rhFlt-3L, rhIL-3 and rhIL-6 for 5 days. At 5 days cells were switched to culture in the equivalent of SFELE medium containing rhEpo, rhSCF and rhIGF-1, with or without 100 μM DMOG, and with or without 1 nM Dex. Total cell number was counted every other day until cell numbers dropped at day 5+19. More than 95% of cells at the end of the culture have erythroblast morphology (data not shown). The graph shows the maximum number of erythroblasts obtained from each condition (n=2). Error bars show one standard deviation.

FIGS. S1 A-B. Dexamethasone promotes formation of larger BFU-E colonies. (A) When scoring BFU-E colonies at day 8-9 we call the large BFU-E-derived colonies with more than 20 CFU-E clusters “early BFU-E colonies” and smaller BFU-E-derived colonies with 5-20 CFU-E clusters “late BFU-E colonies”. Representative early and late BFU-E colonies from cultures with and without 100 nM Dex are shown. When Dex was added to the medium, colonies arising from early BFU-Es were generally larger and contained more CFU-E clusters. (B) Three day CFU-E colony assays were performed in methylcellulose medium containing 10U EPO/mL, with and without 100 nM Dex. In addition to single CFU-E colonies that are formed from CFU-E cells, larger multi-CFU-E colonies formed by earlier progenitor cells were scored. Dex does not affect the size of single CFU-E colonies. Micrographs were acquired by SPOT Advanced v3.5.2 software, using an inverted Nikon Eclipse TS100 microscope, equipped with a RT Monochrome camera. A Nikon 10×/0.25 lens was used in A and a Nikon 20×/0.40 lens was used in B. Scales were derived from pictures of a 0.01 mm objective micrometer.

FIG. S2. Enrichment of BFU-E and CFU-E cells by flow cytometry cell sorting. (A) Day 14.5-15.5 mouse fetal liver cells stained with biotin conjugated antibodies against murine Ter119, B220, Mac-1, CD3, Gr-1, CD32/16, Sca-1, CD41 and CD34 were first depleted by magnetic beads. The enriched negative fraction (FLEP) was then stained with streptavidin-PE, CD71 FITC and CD117 APC antibodies and the PE-negative cells were sorted by FACS into two fractions, called “BFU-E” and “CFU-E”. The “BFU-E” fraction is the 10% lowest CD71 expressing part of the c-kit (CD117)+ fraction. The “CFU-E” fraction is the 20% highest CD71 expressing part of the c-kit fraction. The same FACS setup was later used to separate BFU-E and CFU-Es using CD24a and a combination of CD24a and CD71.

FIG. S3. Morphology of BFU-E and CFU-E cells. Micrographs of sorted CFU-E (c-kit⁺ CD71 and CD24a^{20% high}) cells and BFU-E (c-kit⁺ CD71 and CD24a^{10% low}) cells stained with May-Grünewald Giemsa. BFU-E cells have a high nuclear/cytoplasmic ratio and very fine nuclear chromatin. “CFU-E” cells are larger than “BFU-E” cells with a lower nuclear/cytoplasmic ratio and more regions of heterochromatin. CFU-E cells have multiple, large, well-defined nucleoli (red arrows) in the nuclei. The CFU-E cytoplasm is very basophilic and sometimes bulges out from the cell. Micrographs were acquired by QCapture software, using a Nikon Eclipse E800 microscope, equipped with a QImaging Micro Publisher 3.3 RTV camera. Nikon 100×/0.1.40 and Nikon 20×/0.50 lenses were used. Scales were derived from pictures of a 0.01 mm objective micrometer.

FIGS. S4 A-C. microRNA expression in BFU-E cells treated 4 hours with or without 100 nM Dexamethasone. (A) The read length distribution for solexa reads mapping to known miRNA hairpins is the same for BFU-E cells with and without 100 nM Dex. B) Percent of total miRNA expression of the 20 highest expressed miRNAs. C) Percent of total miRNA expression of the 20 highest expressed miRNA-families.

FIG. S5. As determined by Whole Genome rVISTA, Dexamethasone enhances expression of genes in BFU-E cells enriched for HIF1 and MYC binding sites in their promoters. The figure shows the result of motif enrichment analysis of the 83 genes whose expression in BFU-E cells is increased more than 2-fold in response to Dex. Whole Genome rVISTA recognized 77 of the 83 genes and predicted a very high enrichment of HIF1 and MYC binding sites, conserved from mice to humans, in the 5,000 bp upstream “promoter” regions. The software did not detect significant enrichment of GRE elements. The p value is calculated based on the number of hits in the submitted genomic regions for each motif, compared to the number of conserved hits in the genome. The average base distributions within the called hits for each enriched motif are shown below the table.

FIG. S6. The most enriched motif in promoters of genes upregulated by Dex, as determined by WebMOTIFS, is the shared HIF1 and MYC binding motif caCGTGga. We used WebMOTIFS to analyze “promoter” regions −2000 to +200 bp of the 83 genes upregulated by Dex. The software did not detect significant enrichment of GRE elements. The most enriched motif is caCGTGga, which is both a possible MYC and an HIF1 binding motif.

### DESCRIPTION OF THE TABLES

Table 1.

Dexamethasone prevents BFU-E cell exhaustion, which allows more CFU-E cells to be formed. Sorted “BFU-E” (CD71^{10% low}) cells were cultured in SFELE medium with or without 100 nM Dex. (A) The total number of cells formed from 1000 BFU-E cells at different time points. (B) At different time points CFU-E and BFU-E colony forming assays were performed in the presence of 100 nM Dex. The table shows the number of different colonies formed (±1 standard deviation) per 1000 sorted “BFU-E” cells plated at 0 h. The results demonstrate that Dex reduces differentiation of “BFU-E” cells, which results in increased CFU-E colony formation over time. Standard deviation between four separate biological experiments is shown in parenthesis (n/d=not determined).

Table 2.

DMOG and Dexamethasone synergize to prevent BFU-E cell exhaustion, and enhance CFU-E regeneration. Sorted BFU-E (CD24 and CD71^{10% low}) cells were cultured in SFELE medium with or without 100 nM Dex and with our without 333 μM DMOG. (A) The panel shows the number of cells formed in culture from 1000 BFU-E cells. (B) As in Table 1, CFU-E and BFU-E colony forming assays were performed at 24 hour intervals in the presence of 100 nM Dex. The table shows the number of different colonies formed per 1000 “BFU-E” cells plated at 0 h (±1 standard deviation). The results demonstrate that a combination of DMOG and Dex reduces differentiation and increases self-renewal of “BFU-E” cells, which over time results in increased CFU-E colony formation. While the table shows the average of 4 experiments, CFU-E colony-forming assays at day 7 and 8 were only performed twice.

Table S1.

Efficient fetal liver erythroid progenitor (FLEP) enrichment using only magnetic depletion. Embryonic day 14.5-15.5 mouse fetal livers were resuspended and stained with a lineage-negative (Lin-) cocktail containing biotin conjugated antibodies against murine Ter119 (erythroid), B220 (B cell), Mac-1 (monocyte/granulocyte), CD3 (T cell) and Gr-1 (granulocyte). Biotin conjugated antibodies against CD32/16 (myeloid), Sca-1 (multi-potent progenitors), CD41 (megakaryocyte) and CD34 (myeloid) were sequentially added to the Lin-cocktail to increase the purity of erythroid progenitor cells. The negatively enriched cell populations were subjected to colony forming assays under two different conditions. (A) 1000 cells were plated in methylcellulose medium containing 10U EPO/mL with and without 100 nM Dex. CFU-E colonies were counted 3 days later. (B) 1000 cells were plated in methylcellulose medium containing 10U EPO/mL, 50 ng SCF/mL, 20 ng mIL-3/mL and 20 ng IL-6/mL with and without 100 nM Dex. These cells were cultured 8-9 days before late BFU-E, early BFU-E, CFU-G/M/Mk (granulocyte, monocyte, megakaryocyte), and CFU-GEMM (granulocyte, erythrocyte, monocyte, megakaryocyte) colonies were scored. BFU-Es consisting of a cluster of only 5-20 CFU-Es were scored as late, while larger BFU-Es were scored as early. 2,7-fluorenediamine was used to stain hemoglobin-containing colonies in the plates. (Kaiho and Mizuno, 1985) The table shows the number of colonies per 1000 plated cells. Standard deviation between experiments is shown in parenthesis.

Table S2.

Separation of BFU-E and CFU-E cells by expression of CD71, CD24a or both CD71 and CD24a. C-kit positive BFU-E and CFU-E cells were sorted from FLEP cells based on expression of CD71, CD24a or both CD71 and CD24a. In all experiments the BFU-E fraction was the subset of c-kit^{positive} FLEP cells with the 10% lowest expression of CD71 and/or CD24a, while the CFU-E fraction was the subset with 20% highest expression of CD71 and/or CD24a. Sorted BFU-E and CFU-E populations were subjected to colony forming assays under two different conditions. A fraction of the cells were plated in methylcellulose medium containing 10U EPO/mL with and without 100 nM Dex. The number of CFU-E colonies (±standard deviation) is shown. The sorted “BFU-E” populations mostly formed small CFU-E clusters rather than single CFU-E colonies in these assays. These CFU-E clusters are formed by precursors to CFU-Es and were scored separately as multi-CFU-Es. The number of “multi-CFU-Es” is not shown since these cells likely represent the same cells as those forming small BFU-E colonies.

The potential of the sorted cells to form large BFU-E, small BFU-E, CFU-G/M/Mk, and CFU-GEMM colonies (±standard deviation) was determined by a second assay in methyl cellulose containing 10U Epo, 20 nM IL-3, 20 nM IL-6 and 50 nM SCF per mL, and was scored at day 8-9. Addition of Dex significantly increases the number of large (early) BFU-E colonies (* p-value<0.05, using students t-test). Using both CD71 and CD24a resulted in the most pure BFU-E enrichment. To determine the effect of DMOG, colony assays of sorted BFU-E (CD71 and CD24a^{10% low}) cells were also performed in the presence of 333 μM DMOG. In agreement with the finding that DMOG enhances BFU-E self-renewal BFU-E colonies were larger with a more immature appearance in the presence of DMOG and Dex. Therefore, in the presence of DMOG and Dex most BFU-E colonies were scored as “large BFU-Es.”

Table S3.

The 83 genes expressed more than 2-fold higher in BFU-E cells cultured with 100 nM Dexamethasone. The 83 upregulated genes are listed with relative expression values (RPKM). The score in motif prediction analysis for each gene is listed. In the column listing HIF1 targets predicted by Whole Genome rVISTA we list PubMed ID of publications describing genes (e.g. Egln3=PubMed ID 15823097) (Pescador et al., 2005) as HIF1 targets, even though they were not predicted as such by Whole Genome rVISTA.

### DETAILED DESCRIPTION OF CERTAIN EMBODIMENTS OF THE INVENTION

### I. General

The present invention relates in part to compounds and methods that promote expansion of burst forming unit erythroid (BFU-E) cells. In some aspects, the invention relates to compounds and methods for treating a subject suffering from or at risk of anemia. In some embodiments, the anemia is one that does not respond adequately to administration of Epo. The invention encompasses the recognition that a compound that stimulates erythropoiesis by increasing the number of CFU-E cells would be of use in the treatment of Epo-resistant anemias and bone marrow failure syndromes. The invention also encompasses the recognition that a compound that stimulates erythropoiesis by increasing the number of CFU-E cells could reduce the required Epo dose when Epo is used to treat a subject suffering from or at risk of anemia.

CFU-Es are rapidly dividing cells that are highly responsive to low concentrations of Epo and give rise to erythroblast colonies in ˜7 days (human) or in ˜2 days (mouse) in semi-solid matrices under appropriate culture conditions (see discussion and references in Section II and the Examples). BFU-Es are less mature cells. These cells develop into grouped clusters of erythroblasts or larger colonies (bursts) of more than 500 erythrocytes by ˜15 days (human) or ˜7-10 days (mouse) in culture. As described herein, the inventors developed new techniques to purify BFU-Es and CFU-Es and discovered, among other things, that glucocorticoid receptor agonists, e.g., glucocorticoids (GCs), stimulate BFU-Es to undergo self-renewal. This stimulation increases formation of CFU-E cells and thus the eventual number of differentiated erythroid cells formed from each BFU-E more than 20-fold.

Glucorticoid receptor agonists are steroids or other compounds that bind to the glucocorticoid receptor (GR), a transcription factor expressed by most vertebrate cells that regulates expression of numerous genes by mechanisms that involve binding to sites in DNA called GR response elements (GREs), interacting with coactivator or corepressor proteins, and/or interacting with other transcription factors such as nuclear factor kappa B (NFkB) (Heitzer, M D, et al., Rev Endocr Metab Disord., 8(4):321-30, 2007). The inventors identified genes whose expression is induced in BFU-Es by GCs and analyzed their promoter regions. It was discovered that the promoter regions of genes induced in BFU-Es by treatment with GCs are highly enriched for binding sites for the transcription factors myelomatosis oncogene (Myc) and hypoxia-inducible factor 1 (HIF1) but, surprisingly, not for GR binding sites. HIF-1 is the master regulator of the response to hypoxia in mammals (and various other species) and activates transcription of more than 100 genes involved in adaptation to hypoxic conditions. HIF-1 is composed of two subunits: an ARNT subunit (also called HIF1 beta, abbreviated HIF1b) and HIF1alpha, HIF2alpha or HIF3alpha, abbreviated as HIF1a, HIF2a, and HIF3a, respectively). ARNT assists in DNA binding, while transactivation is mediated through the two transactivation domains on HIF1a or HIF2a, while HIF3a lacks a c-terminal transactivation domain and is thought to mainly inhibit HIF-mediated activation (Lando et al., 2002). It is noted that there are 3 distinct HIF-1 beta chains (ARNT1, 2, and 3, with ARNT1 being most widely expressed). See, e.g., Webb, J D, et al., Cell. Mol. Life Sci., 66:3539-3554 (2009).

Without wishing to be bound by theory, the presence of HIF1 response elements in the promoter regions of most genes upregulated in BFU-Es by GCs suggested to the inventors that GCs and hypoxia, which results in induction of HIF1 activity, (Yei, et al., supra) might cooperate to induce expression of genes that enhance BFU-E self-renewal. Treatment of BFU-Es with a stabilizer of HIF-1 alpha (the prolyl hydroxylase inhibitor dimethyloxalylglycine (DMOG)) enhanced BFU-E self-renewal and led to an increase in CFU-Es, while treatment with a GC (dexamethasone, abbreviated herein as “Dex”), together with DMOG, leads to considerably greater enhancement of BFU-E self-renewal and a dramatic ˜300-fold total increase in production of erythroblasts from each BFU-E.

In some aspects, the present invention relates to the discovery that GR agonists, e.g., GCs, stimulate BFU-E self-renewal, thereby leading to increased production of CFU-Es. In some aspects, the invention relates to the discovery that HIF-1 activators stimulate BFU-E self-renewal, thereby leading to increased production of CFU-Es. In some aspects, invention relates to the discovery that GR agonists, e.g., GCs, and HIF-1 activators can act synergistically to stimulate BFU-E self-renewal, thereby leading to increased production of CFU-Es, with a combined effect that exceeds the sum of the individual effects of these agents. In some aspects, the invention provides methods of expanding BFU-E cells. Certain of the methods comprise contacting one or more BFU-E cells with a HIF-1 activator and a GR agonist. In some aspects, the invention provides methods of treating a subject suffering from or at risk of anemia. Certain of the methods of treatment comprise administering a GR agonist to the subject, wherein the subject has or is at risk of developing an anemia that is not currently treated with a GR agonist such as a GC. In some embodiments, the methods comprise administering a GR agonist and an EpoR agonist (e.g., Epo, an Epo analog such as darbepoetin alpha, or another compound that activates the Epo receptor). Certain of the methods of treatment comprise administering a HIF-1 activator to a subject suffering from or at risk of an anemia, wherein the anemia is an Epo-resistant anemia, anemia caused by a bone marrow failure syndrome, or other anemia that does not respond adequately to Epo or for which Epo administration is not indicated. In some embodiments, the methods comprise administering a HIF-1 activator and a GR agonist to a subject suffering from or at risk of anemia. The invention further provides compositions comprising a HIF-1 activator and a GR agonist. In some embodiments a composition is a pharmaceutical composition.

In some aspects, the invention relates to activating HIF-1, e.g., HIF-1a, in BFU-E cells. In some aspects, the invention relates to contacting BFU-E cells in vitro or in vivo with a compound that activates HIF-1, e.g., HIF-1a, in said cells. In some aspects, the invention relates to inhibiting HIF-1 hydroxylation, e.g., HIF-1 prolyl hydroxylation, e.g., HIF-1a prolyl hydroxylation, in BFU-E cells. In some aspects, the invention relates to contacting BFU-E cells in vitro or in vivo, with a compound that inhibits HIF-1 hydroxylation, e.g., HIF-1 prolyl hydroxylation, e.g., HIF-1a prolyl hydroxylation, in said cells. In some aspects, the invention relates to inhibiting a HIF hydroxylase, e.g., a HIF prolyl hydroxylase, in BFU-E cells. In some aspects, the invention relates to inhibiting HIF-1 hydroxylation, e.g., HIF-1 prolyl hydroxylation, e.g., HIF-1a prolyl hydroxylation, in BFU-E cells. In some aspects, the invention relates to activating or inhibiting various proteins. Proteins discussed herein (e.g., glucocorticoid receptor, HIF1a, HIF2a, HIF3a, HIF-1b, PHD1, PHD2, PHD3, FIH) are well known in the art. If desired, one of skill in the art can readily locate accession numbers and sequences of these proteins (and nucleic acids that encode them) for a species of interest (e.g., human, mouse) using publicly available databases such as those available at the National Center for Biotechnology Information (NCBI) website http://www.ncbi.nlm.nih.gov (e.g., GenBank, Protein, Nucleotide, Gene), incorporated herein by reference. The “Gene” Database provides Gene IDs and reference sequences for such proteins. For example, the following Gene IDs for human proteins are readily found:

HIF-1a—GeneID: 3091

EPAS1 (HIF-2a)—GeneID: 2034

ARNT (HIF-1beta)—GeneID: 405

EGLN2 (PHD1)—GeneID: 112398

EGLN1 (PHD2)—GeneID: 54583

EGLN3 (PHD3)—GeneID: 112399

HIF1AN (FIH)—GeneID: 55662

NR3C1 (GR)—GeneID: 2908

Furthermore, such information is amply described in references herein. In the case of proteins wherein multiple isoforms exist, an activator or inhibitor may activate or inhibit, respectively, one, more than one, or all such isoforms or variants in various embodiments of the invention. In some embodiments, an activator or inhibitor will activate or inhibit, respectively, at least one isoform that is expressed in BFU-Es. In some embodiments, an activator or inhibitor will activate or inhibit, respectively, at least the isoform that is most highly expressed in BFU-Es. In some aspects, an isoform that plays the predominant role in a biological activity of interest (e.g., HIF-1 activity, HIF-1 hydroxylation activity) in BFU-E cells is activated or inhibited.

### II. Purification and Enrichment of EPCs, BFU-Es, and CFU-Es

The invention provides methods of purifying an erythroid progenitor cell population from a population of cells that contains erythroid progenitor cells (EPCs). The invention also provides methods of obtaining a population of cells that is enriched for EPCs relative to a starting population, i.e., contains a higher percentage of EPCs than the starting population. A suitable starting population can comprise, e.g., fetal liver cells, fetal spleen cells, peripheral blood cells (e.g., mobilized peripheral blood cells), bone marrow cells, umbilical cord blood cells, or any population of cells that contains erythroid progenitor cells. Cells can be obtained from a human or animal (e.g., rodent) subject. In some embodiments, the starting population comprises at least 0.1% EPCs. For example, the starting population can contain at least 1%, 2%, 5%, or more EPCs in various embodiments. Sources of, and methods for harvesting, hematopoietic cells are known in the art. Bone marrow cells can be obtained from a source of bone marrow, including but not limited to, ileum (e.g., from the hip bone via the iliac crest), tibia, femora, vertebrate, or other bone cavities. In some embodiments, the invention encompasses expanding BFU-Es that have been obtained through in vitro culture and differentiation of HSCs, embryonic stem (ES) cells, or induced pluripotent stem (iPS) cells. As known in the art, iPS cells are pluripotent cells derived from somatic cells. Such derivation can involve causing somatic cells to contain, e.g., to express, certain transcription factors such as Oct4 and one or more additional transcription factors selected from Sox2, Klf4, Nanog, Lin28, and/or c-Myc, using, e.g., viral vectors, stable or transient transfection, small molecule(s), protein transduction, or combinations thereof. See, e.g., WO/2009/152529 (PCT/US2009/047423); WO/2009/032194 (PCT/US2008/010249); WO/2008/124133 (PCT/US2008/004516). In some embodiments, HSCs are expanded in vitro as described, e.g., in US Pat. Pub. No. 20060115898 (U.S. Ser. No. 11/255,191); US Pat. Pub. No. 20070020757. (U.S. Ser. No. 11/438,847), and/or WO/2008/137641 (PCT/US2008/062365).

In some aspects, an “erythroid progenitor cell” is lineage-restricted such that it gives rise only to erythroid cells. In some embodiments, the methods comprise depleting a population of cells that contains EPCs of cells that are positive for one or more markers that are present at the cell surface of cells that are more mature than CFU-E (e.g., cells at the early proerythroblast to mature erythrocyte stages of development). Suitable markers, e.g., for rodent (e.g., murine) cells include Ter119, CD16, CD32, Sca-1, and CD41. If human cells are to be purified, one or more markers for mature human erythroid cells can be used, which may be different to the markers for murine cells. In some embodiments, cells that are positive for any one or more of the markers are removed from a population. In general, any suitable method known in the art can be used to remove the unwanted (more mature) cells. For example, such depletion can involve contacting a cell population with an agent, e.g., an antibody (e.g., a monoclonal antibody), that binds to the marker. In some embodiments, the antibody may be attached covalently or noncovalently to a support, e.g., a bead, such as a magnetic bead. In some embodiments the antibody may comprise a moiety such as biotin that allows it to be readily attached to a support having an agent that binds to the moiety (e.g., streptavidin) attached thereto. Cells that do not express the marker do not bind to the antibody and can be recovered. For example, cells can be passed through a column containing beads, whereby cells that express the marker become bound to the beads via the antibody, while cells that do not express the marker pass through the column. In some embodiments, cells are incubated in the presence of magnetic beads, whereby the cells expressing the marker become bound to the beads via the antibody, and the beads are then removed using magnetic forces. In other embodiments, the antibody can have a cytotoxic moiety attached thereto. In some embodiments, a depletion step results in removal of at least 80%, 90%, 95%, 98%, 99%, or more of the cells that express a marker of interest, e.g., Ter110, CD16, CD32, Sca-1, or CD41.

The invention provides methods of purifying a BFU-E cell from a population of cells that comprises one or more BFU-E cells. In some embodiments, the starting population comprises at least 0.1% BFU-Es. For example, the starting population can contain at least 1%, 2%, 5%, or more BFU-Es in various embodiments. The invention also provides methods of obtaining a population of cells that is enriched for BFU-Es relative to a starting population, i.e., contains a higher percentage of BFU-Es than the starting population. In some embodiments, a method comprises selecting cells (e.g., from a population of cells that remains after the depletion described above) that are positive for the marker c-kit. In some embodiments, a method for purifying BFU-Es comprises selecting cells (e.g., from a population of cells that remains after the afore-mentioned depletion step) that are within the fraction of cells expressing relatively low or undetectable levels of CD71 (transferrin receptor). For example, in some embodiments, the method comprises selecting cells whose level of expression of CD71 falls within the lowest X % of cells, e.g., within the lowest X % of cells remaining after the depletion step, wherein X is less than or equal to 20. Such cells are referred to herein as CD71^{X % low}. In some embodiments, a method for purifying BFU-Es comprises selecting cells (e.g., from a population of cells that remains after the afore-mentioned depletion step) that are within the fraction of cells expressing relatively low or undetectable levels of CD24a. In some embodiments, the method comprises selecting cells whose level of expression of CD24a falls within the lowest X % of cells, e.g., within the lowest X % of cells remaining after the depletion step, wherein X is less than or equal to 20. Such cells are referred to herein as CD21a^{X % low}. In some embodiments, X (with respect to CD71 and/or CD24a) is between 10 and 20. In some embodiments, X is between 5 and 10. In some embodiments, X is between 1 and 5. In some embodiments, X is less than 1. It will be understood that the value of X can be independently selected for CD71 and CD24a. In some embodiments, the method comprises selecting cells whose level of expression of both CD71 and CD24a falls within the lowest 10% of cells, e.g., within the lowest 10% of cells remaining after the depletion step. In some embodiments, the method comprising steps of: (a) depleting the population of cells that are positive for Ter119, CD16, CD32, Sca-1, and/or CD41; and (b) selecting cells that are (i) c-kit positive and (ii) CD71^{X % low} or CD24a^{X % low}. In some embodiments, step (b) comprises selecting cells that are both CD71^{X % low} and CD24a^{X % low}. In some embodiments, X is 10. The selection can be performed using any method known in the art. For example, cells can be contacted with a binding agent (e.g., an antibody) that binds to CD71 or CD24a, wherein the antibody comprising a fluorescent label. Fluorescence activated cell sorting (FACS) can then be used to isolate cells that that have an expression level within the lower X % for the corresponding marker(s). In some embodiments, wherein the cells are human cells, the method comprises selecting cells that are CD34 positive.

The invention provides methods of purifying a CFU-E cell from a population of cells that comprises one or more CFU-E cells. In some embodiments, the starting population comprises at least 0.1% CFU-Es. For example, the starting population can contain at least 1%, 2%, 5%, or more CFU-Es in various embodiments. The invention also provides methods of obtaining a population of cells that is enriched for CFU-Es relative to a starting population, i.e., contains a higher percentage of CFU-Es than the starting population. In some embodiments, a method comprises selecting cells (e.g., from a population of cells that remains after the depletion described above) that are positive for the marker c-kit. In some embodiments, a method for purifying CFU-Es comprises selecting cells (e.g., from a population of cells that remains after the afore-mentioned depletion step) that are within the fraction of cells expressing relatively high levels of CD71 (transferrin receptor). For example, in some embodiments, the method comprises selecting cells whose level of expression of CD71 falls within the highest X % of cells, e.g., within the highest X % of cells remaining after the depletion step, wherein X is less than or equal to 30. Such cells are referred to herein as CD71^{X % high}. In some embodiments, the method comprises selecting cells whose level of expression of CD24a falls within the highest X % of cells, e.g., within the highest X % of cells remaining after the depletion step, wherein X is less than or equal to 30. Such cells are referred to herein as CD24a^{X % high}. In some embodiments, X (with respect to CD71 and/or CD24a) is between 20 and 30. In some embodiments, X is between 10 and 20. In some embodiments, X is between 5 and 10. In some embodiments, X is between 1 and 5. It will be understood that the value of X can be independently selected for CD71 and CD24a. In some embodiments, the method comprises selecting cells whose level of expression of both CD71 and CD24a falls within the highest 20% of cells, e.g., within the highest 20% of cells remaining after the depletion step. In some embodiments, the method comprising steps of: (a) depleting the population of cells that are positive for Ter119, CD16, CD32, Sca-1, and/or CD41; and (b) selecting cells that are (i) c-kit positive and (ii) CD71^{X % high} or CD24a^{X % high}. In some embodiments, step (b) comprises selecting cells that are both CD7^{X % high} and CD24^{X % high}. In some embodiments, X is 20. The selection can be performed using any method known in the art. For example, cells can be contacted with a binding agent (e.g., an antibody) that binds to CD71 or CD24a, wherein the antibody comprising a fluorescent label. Fluorescence activated cell sorting (FACS) can then be used to isolate cells that have an expression level within the upper X % for the corresponding marker(s).

As known in the art, when cultured in a suitable semi-solid matrix, individual hematopoietic progenitor cells called colony-forming units (CFUs) proliferate to form discrete cell clusters or colonies. CFU assays are typically performed by placing a cell suspension into a semi-solid medium, such as methylcellulose, supplemented with nutrients and cytokines, and incubating at 37 degrees C. for time periods ranging from a few days to several weeks. See, e.g., Miller, C. et al., “Characterization of Mouse Hematopoietic Stem and Progenitor Cells”, Current Protocols in Immunology, Unit 22B.2.1-22B.2.3.1 (2008) and Mouse Colony-Forming Cell Assays Using MethoCult®, Technical Manual, StemCell Technologies, Version 3.1.1 (June 2006), Catalog #28404; Human Colony-Forming Cell Assays Using MethoCult, Technical Manual, StemCell Technologies, Version 3.1.0 (October 2009), Catalog #28404. The CFUs can be classified and quantified based on factors such as the number of cell clusters or colonies, number of cells in a discrete cluster or colony, overall appearance of the cluster or colony, and/or recognition of morphological characteristic of one or more types of hematopoietic lineage cells within the colony. These factors can be assessed using, e.g., light microscopy or other appropriate microscopy/detection methods. Cytochemical and/or immunocytological staining can be performed. The characteristics of cell clusters and colonies derived from CFU-E, BFU-E, and other progenitor cells are well known to those of skill in the art. As described in the Examples, colony forming assays were used to quantify the percentage of BFU-Es and/or CFU-Es in cell populations purified using the inventive methods. Results indicate, for example, that methods of the invention can result in a purified cell population comprising up to 86% BFU-Es, e.g., when X=10 and selection is performed based on CD71, wherein the percentage purity refers to the percentage of viable, colony-forming cells in a cell population. Results indicate, for example, that methods of the invention can result in a purified cell population comprising up to 94% BFU-Es, e.g., when X=10 and selection is performed based on both CD71 and CD24a. It will be understood that selecting lower values of X may result in greater levels of purity. The invention provides a purified cell population comprising at least 75%, 80%, 85%, 90%, 91%, 92%, 93%, 94%, 95%, or more BFU-Es. The invention provides a purified cell population comprising at least 50%, 55%, 60%, 70%, 75%, 80%, 85%, 90%, 91%, 92%, 93%, 94%, 95%, or more CFU-Es. In some embodiments, % purity refers to percentage of viable, colony-forming cells. In some embodiments, cells are mammalian cells. In some embodiments the mammalian cells are rodent cells, e.g., mouse or rat cells. In some embodiments, the cells are primate cells, e.g., human cells.

In some embodiments, a purified cell population (e.g., a population enriched for EPCs, BFU-Es, or CFU-Es) comprises less than about 5% progenitor cells that are less differentiated than BFU-Es and/or are not capable of giving rise to erythroid cells. Such cells include, e.g., CFU-GMM (also termed CFU-G/M/Mk herein), CFU-GM, and CFU-GEMM. For example, the population can comprise less than about 2% of one or more of these cells. For example, the population can comprise less than about 2% CFU-G/M/Mk and/or less than about 1% CFU-GEMM. In some embodiments, the population does not comprise detectable numbers of CFU-GEMM.

The invention provides kits comprising reagents (e.g., antibodies), appropriate for performing an inventive purification technique. In one embodiment, a kit comprises an antibody against CD71 and an antibody against CD24a.

Purified cell populations have a number of uses. For example, such cell populations can be used to test the effect of a compound or condition on BFU-E or CFU-E self-renewal, proliferation, survival, differentiation, apoptosis, or other properties of interest. In some aspects, a purified cell population is useful for identifying culture conditions or compounds, e.g., growth factors, that promote or inhibit a biological response or property of interest as self-renewal, proliferation, commitment, differentiation, or maturation. Certain other are discussed further below. In some aspects, a purified cell preparation is frozen. For example, cells can be frozen at liquid nitrogen temperatures and stored for long periods of time. Once thawed, the cells can be used or expanded, e.g., using methods described herein.

### III. Compositions and Methods Relating to BFU-E Expansion

The invention encompasses the discovery that GCs promote proliferation of BFU-Es, which can result in an increased number of more mature erythroid lineage cells. For example, GC may act at least in part by increasing BFU-E self-renewal. The invention also encompasses the discovery that HIF-1 activators promote proliferation of BFU-Es, which can result in an increased number of more mature erythroid lineage cells. For example, HIF-1 activators may act at least in part by increasing BFU-E self-renewal. The invention further encompasses the discovery that GCs and HIF-1 activators act synergistically to promote BFU-E proliferation, e.g., by increasing self-renewal of BFU-Es. As known in the art, “self-renewal” refers to the ability of certain cells to undergo division and give rise to a progeny cell with the same properties as the parent cell, e.g., having the same developmental potential. In general, self-renewing cell divisions can be symmetric, resulting in two cells having the same properties as the parent cell, or asymmetric, wherein one of the two progeny cells has the same properties as the parent cell while the other cell is typically more differentiated and/or lineage-restricted. It will be appreciated that certain cells have a limited capacity to self-renew. For example, they may undergo several self-renewing cell divisions and then undergo a division in which both progeny cells are more differentiated or lineage-restricted than the parent cell. In some aspects, a compound that increases self-renewal can result in an increased number of cells having the same properties as a parent cell. In some aspects, a compound that increases self-renewal can increase the number of divisions that a cell undergoes before it ceases to self-renew, e.g., before it undergoes a division that results in two cells that have a more restricted developmental potential. In some aspects a compound increases the probability that a BFU-E cell will retain a more immature state during cell division and give rise to daughter cells that are more like the parent BFU-E cell than CFU-E cells. In other words, the compound can help maintain the immaturity of a progenitor cell, e.g., a BFU-E. Over time, an increased number of symmetric or asymmetric BFU-E cell divisions results in an increased number of BFU-E relative to the number that would exist in the absence of the compound. In addition, an increase in the number of asymmetric BFU-E cell divisions results in an increased number of the more differentiated progeny, e.g., CFU-E. This increase in turn results in an increased number of the more mature erythroid cells and, eventually, an increased number of mature, functional RBCs. Certain aspects of the invention accordingly relate to use of GCs or other GR agonists to expand BFU-Es. Certain aspects of the invention relate to use of HIF-1 activators to expand BFU-Es. Certain aspects of the invention accordingly relate to use of GCs (or other GR agonists) in combination with HIF-1 activators to expand BFU-Es. Effects of GCs, other GR agonists, or HIF-1 activators can include enhancing BFU-E self-renewal, maintaining BFU-E immaturity, increasing the number of CFU-Es (or more mature erythroid cells) formed from a population of BFU-Es, increasing the number of mature, functional RBCs formed from a population of BFU-Es, etc.

Compounds of use in various embodiments of the invention, e.g., as HIF-1 activators or GR agonists, can comprise, e.g., small molecules, peptides, polypeptides, nucleic acids, oligonucleotides, etc. A small molecule is often an organic compound having a molecular weight equal to or less than 2.0 kD, e.g., equal to or less than 1.5 kD, e.g., equal to or less than 1 kD, e.g., equal to or less than 500 daltons and usually multiple carbon-carbon bonds. Small molecules often comprise one or more functional groups that mediate structural interactions with proteins, e.g., hydrogen bonding, and typically include at least an amine, carbonyl, hydroxyl or carboxyl group, and in some embodiments at least two of the functional chemical groups. A small molecule may comprise cyclic carbon or heterocyclic structures and/or aromatic or polyaromatic structures substituted with one or more chemical functional groups and/or heteroatoms.

In some embodiments, a compound of use in the invention comprises a nucleic acid, e.g., an oligonucleotide (which refers to short nucleic acids, e.g., 50 nucleotides in length or less). A nucleic acid can be single-stranded, double-stranded (ds), blunt-ended, or double-stranded with one or more overhang(s). In some embodiments a compound is an RNAi agent, antisense oligonucleotide, or aptamer. The term “RNAi agent” encompasses nucleic acids that can be used to achieve RNA silencing, e.g., RNA interference (RNAi), in mammalian cells. RNAi agents include short interfering RNA (siRNA), short hairpin RNA (shRNA), microRNA (miRNA) and miRNA precursors. A nucleic acid may contain one or more non-standard nucleotides, modified nucleosides (e.g., having modified bases and/or sugars) or nucleotide analogs, and/or have a modified backbone. Any modification or analog recognized in the art as useful for RNAi, aptamers, antisense, or other uses of oligonucleotides can be used.

In some embodiments, a compound comprises a polypeptide. Polypeptides may contain any of the 20 amino acids that are naturally found in proteins and are genetically encoded (“standard” amino acids), other amino acids that are found in nature, and/or artificial amino acids or amino acid analogs. One or more of the amino acids in a polypeptide may be modified, for example, by the addition of a chemical entity such as a carbohydrate group, a phosphate group, a fatty acid group, an alkyl group, etc.

In some embodiments, a compound of use in one or more embodiments of the invention is approved by the Food & Drug Administration (FDA) or by an agency responsible for regulating pharmaceutical agents in a country or region other than the US, for treatment of at least one disease or condition in humans. In some embodiments, a compound has undergone at least a Phase I clinical trial. In some embodiments, a compound has undergone at least a Phase II clinical trial. In some embodiments, a compound has undergone a Phase III clinical trial.

In some embodiments, a compound (e.g., an activator) is used at a concentration or in an amount that increases the amount (e.g., concentration) or activity of the molecular entity to be activated (which may be referred to as a “target”) by a desired amount. The amount or activity may be increased, e.g., by a factor of at least about 1.25 (i.e., a 25% increase), 1.5, 2, 5, 10-fold, or more relative to a reference value. A reference value can be, e.g., a level that represents amount or activity of the target in the absence of the activator. One of skill in the art will be able to perform appropriate assays to measure an increase in amount or activity using appropriate methods depending upon the particular target. It will be understood that an increase in total measured activity can result from (i) an increase in amount (increased number of target molecule), (ii) an increase in average activity per target molecule, or a combination of (ii) and (iii).

It will be understood that terms such as “inhibit”, or “inhibition” (and similar terms such as “reduce”, “reduction”, or “decrease”) often refers to partial rather than complete (100%) inhibition. In some embodiments, an inhibitor is used at a concentration or in an amount that inhibits (reduces) the amount (e.g., concentration) or activity of the molecular entity to be inhibited (which may be referred to as a “target”) by a desired amount. The inhibition may be at least about 5%, 10%, 15%, 20%, 25%, 30%, 35%, 40%, 45%, 50%, 55%, 60%, 65%, 70%, 75%, 80%, 85%, 90%, 95%, 99%, or 100% of a reference value. A reference value can be, e.g., a level that represents amount or activity of the target in the absence of the inhibitor. One of skill in the art will be able to perform appropriate assays to measure a decrease in amount or activity using appropriate methods depending upon the particular target. It will be understood that 100% inhibition can refer to reduction to a background or undetectable level. It will be understood that a decrease in total measured activity can result from (i) a decrease in amount of target molecule (decreased number of target molecule), (ii) a decrease in average activity per target molecule, or a combination of (ii) and (iii).

HIF-1 Activators

As used herein, “HIF-1 activator” refers to a compound that increases (enhances, promotes) HIF-1 activity, e.g., in a cell contacted with the compound. In some aspects, “HIF-1 activity” refers to binding of HIF-1 to characteristic HIF-1 binding sites in DNA (e.g., containing a consensus sequence RCGTG) whereby HIF-1 can regulate, e.g., activate, transcription of operably associated transcription units. A HIF-1 activator can act by any of a variety of mechanisms. Such mechanisms could include, for example, any mechanism that results in increased levels of HIF-1, e.g., by causing increased expression or reduced degradation of a HIF alpha subunit. In some embodiments, a HIF-1 activator increases activity of HIF-1 that comprises HIF-1a and HIF-1b. In some embodiments, a HIF-1 activator increases activity of HIF-1 that comprises HIF-2a and HIF-1b. In some embodiments, a HIF-1 activator increases activity of both HIF-1 that comprises HIF-1a (and HIF-1b) and HIF-1 that comprises HIF-2a (and HIF-1b).

Any of a variety of HIF-1 activators are of use in various embodiments of the invention. HIF-1 activation in hypoxia involves a variety of different processes including HIF-alpha chain synthesis, stabilization, nuclear import, dimerization, DNA binding, and co-activator recruitment (reviewed in Webb, J D, et al., supra and Koh, M Y, et al., Trends in Biochem. Sci. 33(11): 526-534 (2008)). In some embodiments, a HIF-1 activator is a compound that mimics the effect of hypoxia on a cell. Compounds that induce or mimic endogenous mechanisms of HIF-1 activation can be used to increase HIF-1 activity. Under normoxic conditions, HIF-alpha chains are rapidly degraded by the proteasome following ubiquitylation at various sites by an E3 ligase that contains the von Hippel-Lindau protein (pVHL) as the substrate recognition component. Recognition of HIF-alpha by pVHL is facilitated by hydroxylation of either or both of two proline residues within HIF alpha chains (e.g., proline 402 and 564 in human HIF-1a protein). In some embodiments of the invention, a HIF-1 activator is a compound that inhibits hydroxylation of HIF-1. HIF-1 amino acid hydroxylations are performed by members of a family of 2-oxoglutarate and iron (II) dependent dioxygenases referred to as “HIF hydroxylases”. The mammalian genome encodes three HIF prolyl hydroxylases that have been shown to affect HIF-1 activity: PHD1, PHD2, and PHD3 (also referred to as Egln1, 2 and 3) (Jaakkola et al., 2001). A reduction in HIF prolyl hydroxylase activity allows HIF-1 to escape from destruction, thereby resulting in increased HIF-1 activity.

The interaction between HIF-1 and pVHL is further accelerated by acetylation of a lysine residue (at position 532 in human HIF-1a) by an N-acetyltransferase (ARD1). In some embodiments of the invention, a HIF-1 activator is a compound that inhibits synthesis, stability, or activity of ARD1, thus promoting escape of HIF alpha from destruction. Recruitment of co-activator(s), e.g., p300/CBP (and thus transcription activating activity) is blocked by an oxygen-dependent hydroxylation of an asparaginyl residue in the C-terminal activation domain of HIF-1 and HIF-2 alpha chains (residue 803 in human HIF-1a). The mammalian genome encodes a single HIF asparaginyl hydroxylase, termed factor-inhibiting HIF (FIH). A reduction in FIH activity allows HIF-1 to escape from blockade of co-activator recruitment, thereby resulting in increased HIF-1 activity.

In some embodiments of the invention, a HIF-1 activator is a compound that inhibits synthesis, stability, or activity of a prolyl hydroxylase. Such compounds are referred to herein as prolyl hydroxylase inhibitors (PHIs). In some embodiments, the PHI is a HIF PHI, i.e., a PHI that inhibits PHD 1, 2, and/or 3. In some embodiments of the invention, a HIF-1 activator is a compound that inhibits FIH synthesis, stability, or activity.

A variety of PHIs are known in the art and can be used in certain embodiments of the invention. In some embodiments, a PHI binds to a HIF hydroxylase (e.g., a HIF PHD) and inhibits its enzymatic activity. Exemplary PHIs are described, e.g., in WO/2010/022240 (PCT/US2009/054473); WO/2010/018458 (PCT/IB2009/006751); WO/2009/134754 (PCT/US2009/041908); WO/2009/134750 (PCT/US2009/041902); WO/2009/129945 (PCT/EP2009/002693); WO/2009/089547 (PCT/US2009/030775); WO/2009/073669 (PCT/US2008/085286); WO/2007/146438 (PCT/US2007/014157); WO/2007/146425 (PCT/US2007/014077); WO/2007/115315 (PCT/US2007/065987); WO/2007/090068 (PCT/US2007/061171); WO/2006/133391 (PCT/US2006/022403); WO/2006/094292 (PCT/US2006/008117); WO/2004/108121 (PCT/US2004/017772); WO/2003/053997 (PCT/US2002/039163); WO 2003/049686 (PCT/US2002/038867); WO/2008/137084 (PCT/US2008/005698); WO/2008/137060 (PCT/US2008/005664); WO/2008/130600 (PCT/US2008/004965); WO/2008/130508 (PCT/US2008/004634); WO/2008/076427 (PCT/US2007/025819); WO/2008/076425 (PCT/US2007/025799); WO/2008/002576 (PCT/US2007/014832); WO/2007/070359 (PCT/US2006/046785); WO/2003/080566 (PCT/GB2003/001239); WO/2007/086663 (PCT/KR2007/000242) {

In some embodiments, a HIF PHI is a structural mimetic of 2-oxoglutarate. Exemplary compounds are described, e.g., in WO/2005/011696 and references therein. Exemplary compounds include, e.g., N-((1-chloro-4-hydroxy-isoquinoline-3-carbonyl)-amino)-acetic acid, [(7-Bromo-4-hydroxy-isoquinoline-3-carbonyl)-amino]-acetic acid, [(1-Chloro-4-hydroxy-7-methoxy-isoquinoline-3-carbonyl)-amino]-acetic acid, [(7-Chloro-3-hydroxy-quinoline-2-carbonyl)-amino]-acetic acid, [(3-Hydroxy-6-isopropoxy-quinoline-2-carbonyl)-amino]-acetic acid, and [(4-Hydroxy-7-phenylsulfanyl-isoquinoline-3-carbonyl)-amino}-acetic acid, and dimethyloxalylglycine (DMOG). Other 2-oxoglutarate mimetics are described in Mole, D., et al., Bioorganic & Medicinal Chem. Lett. 13: 2677-2680 (2003).

In some embodiments, a HIF PHI is the compound referred to as FG-2216, the compound referred to as FG-4539, or the compound referred to as FG-4592 (Fibrogen, licensed to Astellas). See, e.g., Hsieh M M, et al. HIF prolyl hydroxylase inhibition results in endogenous erythropoietin induction, erythrocytosis, and modest fetal hemoglobin expression in rhesus macaques. Blood. 110(6):2140-2147 (2007); Bernhardt W, Wiesener M S, Schmieder R E, Gunzler V, Eckardt K U. The Prolyl Hydroxylase Inhibitor FG-2216 Stimulates EPO Production in Nephric and Anephric Dialysis Patients—Evidence for an Underutilized Production Capacity in Liver and Kidneys. Vol. 2007. San Francisco: American Society of Nephrology Renal Week; 2007. Nov. 2-5, Abstract: SA-PO784; Frohna P, et al., Results from a Randomized, Single-Blind, Placebo-Controlled Trial of FG-4592, a Novel Hypoxia Inducible Factor Prolyl Hydroxylase Inhibitor, in CKD Anemia. Vol. 2007. San Francisco American Society of Nephrology Renal Week; 2007. Nov. 2-5, Abstract SU-PO806.

In some embodiments, a HIF PHI inhibits synthesis, stability, or activity of one or more additional 2-oxoglutarate-dependent dioxygenases in addition to inhibiting at least one PHD. For example, in some embodiments a HIF PHI inhibits FIH. In some embodiments, a PHI inhibits a HIF PHD, and shows little to no inhibitory activity (e.g., has an IC50 at least times as great) towards most or all other 2-oxoglutarate-dependent dioxygenases. For example, in some embodiments, a PHI inhibits a HIF PHD but shows little or no inhibitory activity towards procollagen lysyl hydroxylase, procollagen prolyl 3-hydroxylase, and/or procollagen prolyl 4-hydroxylase. In some aspects, a HIF PHI inhibits a prolyl hydroxylase that plays the predominant role in hydroxylating HIF-1a in BFU-Es, e.g., in hypoxic conditions.

In some embodiments, a PHI comprises an oligonucleotide. In some embodiments, the PHI comprises an RNAi agent, e.g., an siRNA that inhibits expression of a HIF PHD, e.g., PHD1, PHD2, and/or PHD3. In some embodiments, the PHI inhibitor comprises an aptamer that binds to a HIF PHD and inhibits its activity.

In some embodiments, a PHI comprises a polypeptide. A polypeptide can be, e.g., a dominant negative version of a PHI. In some embodiments, a polypeptide that binds to and inhibits activity of a PHI is selected, e.g., from a library such as a phage display or ribosome display library.

OS-9 is a protein that interacts with both HIF-1a and HIF-1a prolyl hydroxylases. Overexpression of OS-9 promotes the hydroxylation of HIF-1a, HIF-1a binding to pVHL, proteasomal degradation of HIF-1a, and loss of HIF-1-mediated transcription. In some embodiments, a HIF-1 activator is a compound that inhibits the activity, synthesis, or stability of OS-9 or inhibits interaction of OS-9 with HIF-1 or 1-HIF-1 PHD. See, e.g., WO/2006/009843 (PCT/US2005/021461).

In some embodiments, a HIF activator is a compound that inhibits synthesis, stability, or activity of FIH. Such compounds are referred to as “FIH inhibitors”. N-oxaloylglycine (NOG) is an exemplary compound that inhibits FIH activity. Inhibitor N-oxalyl-d-phenylalanine was shown to inhibit the FIH but not a HIF prolyl hydroxylase (McDonough, M A, et al, J Am Chem Soc., 127(21):7680-1 (2005). Availability of a crystal structure facilitates identification of additional compounds that inhibit FIH enzymatic activity. See, e.g., WO/2004/035812 (PCT/GB2003/004492); Lee, C., et al., J Biol Chem. 278(9):7558-63 (2003); Dann, C E, Proc Natl Acad Sci USA, 99(24):15351-6 (2002).

In some embodiments, a PHI, OS-9 inhibitor, or FIH inhibitor is an RNAi agent (e.g., siRNA) that inhibits expression of a PHI, OS-9, or FIH. In some embodiments, a PHI, OS-9 inhibitor, or FIH inhibitor is an aptamer that binds to a PIH, OS-9, or FIH and inhibits its activity.

GR Activators

As used herein, the term “GR activator” refers to a compound that increases (enhances, promotes) GR activity. In some aspects, “GR activity” refers to ability of GR to bind to characteristic GR binding sites in DNA and, optionally, regulate transcription of operably associated transcription units. In some aspects, “GR activity” refers to ability of GR to bind to a DNA-associated transcription factor and, optionally, regulate transcription of an operably associated transcription unit. In some aspects, “GR activity” refers to ability of GR to cause an increase in the expression of one or more genes listed in Table S3, e.g., in BFU-Es. In some embodiments, a GR activator binds directly to GR. As used herein, such compounds will be referred to as “GR agonists”. In some embodiments, a GR activator promotes displacement of a chaperone such as Hsp90 from GR. In some embodiments, a GR agonist promotes translocation of GR from the cytoplasm to the nucleus.

In some embodiments, a GR agonist is a GC. A GC can be a naturally occurring steroid hormone such as cortisol or a synthetic GC such as dexamethasone or prednisone. A “naturally occurring GC” refers to a GC having a structure found in nature, but it will be understood that compound may be synthesized by man. A “synthetic GC” refers to a steroid compound having a structure created by man that binds to the GR. Typically such compounds have at least one substituent that differs from that found at the corresponding position of a naturally occurring GC. In some embodiments, a synthetic GC has a potency and/or binding affinity for the GR at least as great as that of cortisol. Potency and/or binding affinity can be measured using methods known in the art. For example, a cell-free competitive radio-labeled GR binding assay can be used. GR-mediated transcriptional activity can be assessed using a reporter gene assay. Potency can be assessed in vivo, e.g., an ACTH suppression test can be used. Numerous GCs are known in the art. See, e.g., Burger's Medicinal Chemistry and Drug Discovery, Goodman & Gilman, Goodman and Gilman's The Pharmacological Basis of Therapeutics, Basic and Clinical Pharmacology, McGraw-Hill/Appleton & Lange; 11th edition (July 2009); Fauci, et al. (eds.) Harrison's principles of internal medicine, 17th ed. New York: McGraw-Hill, 2008, Williams Textbook of Endocrinology. Exemplary GCs include cortisone, hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-7-butyrate, hydrocortisone-17-valerate, aclomethasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, fluticasone, fluticasone furoate, and fluticasone propionate. It will be understood that in various embodiments such compounds may optionally be provided as an ester or associated with a counterion. In some embodiments, the compound binds to substantially the same site as does a naturally occurring or synthetic GC such as dexamethasone. See, e.g., Bledsoe, R K, et al, Cell, 110, 93-105 (2002) for a description of the crystal structure of the GR ligand binding domain in a complex with Dex.

In some embodiments, a GR agonist is a selective glucocorticoid receptor agonist (SEGRA). In some embodiments, a “SEGRA” is a GR agonist that has reduced transactivation activity of genes whose activation is at least in part responsible for deleterious side effects associated with administration of certain GC, e.g., prednisone or Dex. In some embodiments, conversely, a “SEGRA” is a GR agonist that has reduced transrepression activity of genes whose repression is at least in part responsible for deleterious side effects associated with administration of certain GCs, e.g., prednisone or Dex. See, e.g., Schäcke, H, et al., “Selective glucocorticoid receptor agonists (SEGRAs): novel ligands with an improved therapeutic index.”. Molecular and cellular endocrinology 275 (1-2): 109-17; Rehwinkel, H and Schäcke H., GR ligands: can we improve the established drugs. ChemMedChem. (8):803-5 (2006). In some embodiments, the GR agonist is not a SEGRA.

In some embodiments, a GR agonist is a non-steroid compound that binds to the GR. In some embodiments, the compound binds to substantially the same site as does a naturally occurring or synthetic GR ligand such as dexamethasone while in other embodiments the GR agonist binds to a different site on the GR. See, e.g., Rehwinkel, H and Schäcke H, supra, Biggadike, K., et al. PNAS, 106(43), 18114-18119 (2009), and references therein, for exemplary compounds.

### IV. Applications and Further Aspects and Embodiments

Compositions and methods of the invention may be used in vitro and/or in vivo for a variety of purposes. In some embodiments, compositions and methods of the invention are used in vitro, e.g., for research or clinical purposes (e.g., determining the potential responsiveness of a subject's condition to a compound or compound combination, examining the effect of a compound or compound combination on a cell, identifying or testing compounds (e.g., screening assays)). For example, a purified population of BFU-E or CFU-E can be used to screen compound libraries, e.g., to identify compounds and compound combinations that are particularly effective in promoting BFU-E self-renewal, CFU-E generation, or any other process of interest., or to identify genes that play a role in promoting BFU-E self-renewal, CFU-E generation, or any other process of interest. Compounds to be screened can come from any source, e.g., natural product libraries, combinatorial libraries, libraries of compounds that have been approved by the FDA or another health regulatory agency for use in treating humans, siRNA, shRNA, etc. In some aspects, a “library” is a collection of compounds, optionally arranged in a standardized format (e.g., in multiwall plates). In some embodiments, a library comprises a set of compounds that has been selected based on having particular structural or functional properties. For example, a library could be selected to comprise one or more GC receptor agonists, growth factors, or PHIs. In some embodiments, a library comprises at least 10 different GC receptor agonists, growth factors, and/or PHIs.

The method may encompass performing high througput screening, e.g., using multiwell plates, liquid handling robots, etc. In some embodiments at least 100; 1,000; or 10,000 compounds are tested. Compounds identified as “hits” can then be tested in further assays, e.g., to assess their effect on BFU-E self-renewal, CFU-E generation etc. For example, a first screen could identify compounds that promote an increase in cell number, while a second screen could more specifically determine whether the compound results in an increased number of BFU-E, CFU-E, and/or more differentiated erythroid lineage cells. Compounds identified as having a useful effect can be selected and systematically altered, e.g., using rational design, to optimize binding affinity, avidity, specificity, or other parameters. For example, one can screen a first library of compounds, identify one or more compounds that are “hits” or “leads”, and subject those hits to systematic structural alteration to create a second library of compounds structurally related to the hit or lead. The second library can then be screened using the methods described herein or other methods known in the art. A compound can be modified or selected to achieve (i) improved potency, (ii) decreased toxicity and/or decreased side effects; (iii) modified onset of therapeutic action and/or duration of effect; and/or (iv) modified pharmacokinetic parameters (absorption, distribution, metabolism and/or excretion). In some embodiments, purified EPCs, BFU-Es, and/or CFU-Es are cultured in medium containing a GR agonist (e.g., Dex or prednisone). Multiple HIF-1 activators are screened to identify one or more compounds that have particularly strong synergy with that GR agonist. In other embodiments, purified EPCs, BFU-Es, and/or CFU-Es are cultured in medium containing a HIF-1 activator (e.g., a PHI). Multiple GR agonists are screened to identify one or more compounds that have particularly strong synergy with that HIF-1 activator.

In certain embodiments, the survival and/or proliferation of a cell or cell population is determined by an assay selected from: a cell counting assay, a replication labeling assay, a cell membrane integrity assay, a cellular ATP-based viability assay, a mitochondrial reductase activity assay, a caspase activity assay, an Annexin V staining assay, a DNA content assay, a DNA degradation assay, and a nuclear fragmentation assay. Other exemplary assays include BrdU, EdU, or H3-Thymidine incorporation assays; DNA content assays using a nucleic acid dye, such as Hoechst Dye, DAPI, Actinomycin D, 7-aminoactinomycin D or Propidium Iodide; Cellular metabolism assays such as AlamarBlue, MTT, XTT, and CellTitre Glo; Nuclear Fragmentation Assays; Cytoplasmic Histone Associated DNA Fragmentation Assay; PARP Cleavage Assay; TUNEL staining; and Annexin staining.

Cells can be cultured in the presence of compound(s) for various periods of time prior to assessing the cells. In certain embodiments cells are cultured for between 12 hours and 30 days, e.g., for between 1 and 20 days, for between 3 and 10 days, or any intervening range or particular value.

In some embodiments, compositions and methods of the invention are used in vitro for expanding BFU-Es. For example, BFU-Es are cultured in medium containing a HIF-1 activator, a GR agonist, or both. The resulting cell population (or a purified fraction thereof) can be administered to a subject in need thereof, e.g., a subject suffering from or at risk of anemia. In some embodiments, BFU-Es for use in the ex vivo expansion methods are obtained from the individual to whom the resulting expanded cells are to be administered. For example, BFU-Es could be obtained from a subject prior to the subject receiving chemotherapy or other therapy (medical or surgical) expected to cause anemia or significant blood loss and cultured in medium containing a HIF-1 activator, GR agonist, or both. At least some of the resulting cells are administered to the subject, e.g., prior to, during, or after a course of chemotherapy or prior to, during, or after surgery, etc. Cells can be administered to a subject by standard means, such as intravascularly, e.g., by intravenous infusion. In other embodiments, cells are administered to an individual by infusion into an artery, e.g., the superior mesenteric artery or celiac artery. The number of cells administered can range, e.g., from thousands to many millions. In some embodiments, between 10⁵ and 10¹³ cells are administered.

In various embodiments BFU-Es are cultured in medium containing a HIF-1 activator, a GR agonist, or both, for at least 2 days, e.g., at least 3, 5, 7, or 10 days. In some embodiments, cells are cultured for up to about 2, 3, 4, 6, or 8 weeks, or more. In some embodiments, cells are cultured for between 7 days and 15 days, or between 7 days and 30 days. Any culture medium suitable for maintaining hematopoietic cells can be used in various embodiments of the invention. Various media known in the art can be used for the expansion of BFU-Es. As known in the art, culture media suitable for mammalian cells typically contain sugars, amino acids, lipids, salts, vitamins, and various other components necessary or helpful to support cell survival and proliferation. In some embodiments, a medium is a liquid medium. Exemplary liquid media include Dulbecco's MEM and Iscove's Modified Dulbecco's Medium (IMDM). In some embodiments, the cell culture medium is serum free. One serum free medium which can be used in the methods of the invention is serum free StemSpan® SFCM (Stem Cell Technologies). Media can contain various growth factors. In some embodiments, a base medium (e.g., a growth factor-free medium) is supplemented with one or more growth factors. In some embodiments, media contains one or more growth factors selected from the group consisting of: Epo, IL-3, IL-6, stem cell factor (SCF), insulin-like growth factor (IGF), e.g., IGF-1, and GM-CSF. Media can contain 1, 2, 3, 4, or more factors in various embodiments. For example some embodiments, media contains Epo, IGF-1, and SCF. In some embodiments, the concentrations of growth factors used ranges from about 0.1 ng/mL to about 500 ng/mL, e.g., from about 5 ng/mL to about 200 ng/mL, e.g., from about 10 or 20 ng/mL to about 50 ng/mL or about 100 ng/mL. In some embodiments, the concentration of a growth factor, e.g., Epo, is expressed in units (U). (See, e.g., Jelkmann, W., et al., Nephrology Dialysis Transplantation 24(5):1366-1368, 2009, and references therein, for discussion of units applicable to Epo, e.g., recombinant human Epo (rhEpo)). In some embodiments, about 1-5 U/mL Epo is used, e.g., about 2 U/mL Epo. In some embodiments, the medium contains about 100 ng/mL SCF, about 40 ng/mL IGF-1 and about 2 U/mL Epo. In some embodiments, a growth factor is a human protein (the protein has the sequence of a protein that is encoded by the human genome). In some embodiments, a growth factor is a mouse protein (the protein has the sequence of a protein that is encoded by the human genome). In some embodiments, a growth factor used in the culture of human cells is a human protein. Growth factors can be produced using methods known in the art. In some embodiments, a growth factor is recombinantly produced by a suitable host cell, e.g., a Chinese hamster ovary (CHO) cell. In some embodiments, a growth factor is purified from cells or tissues that naturally produce it. Many growth factors can be obtained from commercial suppliers. In some embodiments, a suitable growth factor is a molecule having similar or equivalent biological activity to the growth factors of interest herein. Such molecules typically bind to the same receptor as a growth factor of interest. In some aspects, a polypeptide whose sequence comprises or consists of a sequence at least 90%, 95%, 98%, 99% or more identical to the sequence of a growth factor of interest, is of use and can be considered equivalent to a growth factor of interest. In some aspects, a polypeptide whose sequence has no more than 1, 2, 3, 4, or 5 amino acid changes relative to sequence of a growth factor of interest, is used. Suitable variants are known in the art or can be readily identified without undue experimentation. In some aspects, a polypeptide whose sequence comprises or consists of a sequence at least 90%, 95%, 98%, 99% or more identical to a receptor-binding portion of a growth factor is of use. In some embodiments, a non-polypeptide growth factor receptor agonist is of use.

In most aspects, suitable culture conditions comprise culturing at about 33 degrees C. to about 39 degrees C., e.g., at about 37 degrees C. In some embodiments the oxygen concentration is about 4% to about 20%. The medium can be replaced in whole or in part throughout the culture period. For example, in some embodiments, half of the medium is replaced twice per week with fresh media. Any suitable expansion container, flask, or appropriate tube or vessel such as a 12, 24 or 96 well plate, T flask or gas-permeable bag can be used in the culture methods of the invention. Such culture containers are commercially available, e.g., from Falcon, Coming or Costar. As used herein, “expansion container” also is intended to include any chamber or container for expanding cells whether or not free standing. Cell culture media suitable for culturing EPCs, wherein the media contain a HIF-1 activator and, optionally, a GR agonist, are an aspect of the invention. In some embodiments, the invention provides a cell culture medium containing Epo and a HIF-1 activator. In some embodiments, the invention provides a culture medium containing Epo, a HIF-1 activator, and a GR activator. In some embodiments, the invention provides a culture medium containing Epo, a HIF-1 activator, a GR activator, and at least one growth factor selected from IGF (e.g., IGF-1) and SCF. In some embodiments, the GR activator is Dex. In some embodiments, a GR activator, e.g., Dex, is used at a concentration of between 1 pM and 10 μM, e.g., between 0.01 nM or 0.1 nM and 500 nM or 1 μM, e.g., between 1 nM and 500 nM, e.g., between about 10 nM and about 250 nM, e.g., about 100 nM. In some embodiments, a GR agonist that has a higher potency than Dex may be used at a lower concentration. In some embodiments, a GR agonist that has a lower potency than Dex may be used at a higher concentration. In some embodiments, a HIF-1 activator is used at a concentration of between 1 pM and 1 mM, e.g., between 0.01 nM and 500 μM, e.g., between 0.1 nM and 10 μM, e.g., between 1 nM and 1 μM. One of skill in the art will readily be able to determine appropriate concentrations. Such ranges may be used in screening methods of the invention.

In some embodiments, hematopoietic progenitor cells are obtained from a population of cells comprising human CD34⁺ cells. In some embodiments, human CD34+ cells are obtained from bone marrow cells, umbilical cord blood cells, peripheral blood cells, and fetal liver cells. In some embodiments, peripheral blood cells are mobilized peripheral blood cells. In some embodiments, human CD34+ cells are cultured in medium (e.g., serum-free medium) supplemented with one or more cytokines selected from SCF, Flt-3L (Flt-3 ligand), IL-3 and IL-6 (e.g., at least 2, 3, or all 4 of these) for, e.g., about 4-10 days, e.g., 5 days, and then cultured in medium that contains a HIF-1a activator and a GR agonist. In some embodiments, such medium comprises one or more factors such as Epo, IL-3, IL-6, stem cell factor (SCF), insulin-like growth factor (IGF), e.g., IGF-1, and GM-CSF as described above. For example, in some embodiments, media contains Epo, IGF-1, and SCF. In some embodiments, the culture medium comprises between 20 μM and 500 μM DMOG and between 0.2 nM and 5 nM Dex. In some embodiments, the culture medium comprises between 50 μM and 250 μM DMOG and between 0.1 nM and 2.5 nM Dex. In some embodiments, the culture medium comprises about 100 μM DMOG and about 1 nM Dex.

In some embodiments, culturing a population of cells comprising human hematopoietic progenitor cells in medium comprising a HIF-1a activator (e.g., a PHI, e.g., DMOG) and a GR agonist (e.g., a glucocorticoid, e.g., Dex) results in an increase of at least about 3-fold, e.g., between about 3-fold and about 5-fold (e.g., about 4-fold) in the number of erythroblasts as compared with culturing such cells in the same medium but without the GR agonist. In some embodiments, culturing a population of human hematopoietic progenitor cells in medium comprising a HIF-1a activator and a GR agonist results in an increase of at least about 8-fold, e.g., between about 8-fold and about 12-fold (e.g., about 10-fold) in the number of erythroblasts as compared with culturing such cells in the same medium but without the HIF-1a activator. In some embodiments, culturing a population of human hematopoietic progenitor cells in medium comprising a HIF-1a activator and a GR agonist results in an increase of at least about 15-fold, e.g., between about 15-fold and about 25-fold, in the number of erythroblasts as compared with culturing such cells in the same medium but lacking the HIF-1a activator and GR agonist. In some embodiments, an increase in erythroblasts of such magnitude is evident after, e.g., between about 5 days and about 30 days of culture, e.g., after about 20 days in culture in the medium. In some embodiments, the population of cells comprises at least about 80% erythroblasts, e.g., between 80% and 99% or 100% erythroblasts, e.g., between 85% and 95%, 96%, 97%, 98%, 99%, or 100% or between 90% and 95%, 96%, 97%, 98%, 99%, or 100%, following culture in the medium.

In some aspects, the invention relates to or makes use of genetically modified cells. For example, ESCs, BFU-Es, or CFU-Es purified or generated according to the instant invention can be genetically modified. Such cells can be genetically modified in culture or, in the case of non-human cells, can be derived from a genetically modified animal. A “genetically modified” or “engineered” cell refers to a cell into which a nucleic acid has been introduced by a process involving the hand of man (or a descendant of such a cell that has inherited at least a portion of the nucleic acid). The nucleic acid may for example contain a sequence that is not naturally found in the cell, it may contain native sequences (i.e., sequences naturally found in the cell) but in a non-naturally occurring arrangement (e.g., a coding region linked to a promoter from a different gene), or altered versions of native sequences, etc. The process of transferring the nucleic acid into the cell can be achieved by any suitable technique and will often involve use of a vector (e.g., as discussed below). In some embodiments the nucleic acid or a portion thereof is integrated into the genome of the cell and/or is otherwise stably heritable. The nucleic acid may have subsequently been removed or excised from the genome, provided that such removal or excision results in a detectable alteration in the cell relative to an unmodified but otherwise equivalent cell. For example, the cell may have been engineered to express a growth factor, growth factor receptor, oncogene, transcription factor, enzyme, microRNA, shRNA, antisense molecule, etc., or to have reduced or absent expression of an endogenous gene.

In some aspects, the invention relates to or makes use of genetically modified multi-cellular vertebrate organisms. An organism at least some of whose cells are genetically engineered or that is derived from such a cell is considered a genetically engineered organism. Such an organism may be a non-human mammal. In some embodiments, the organism may serve as an animal model for anemia or a disorder that can result in anemia. For example, the subject may be a genetically engineered mouse. In some embodiments, the mouse is a knockout mouse.

In some aspects, a cell or organism is genetically modified using a suitable vector. As used herein, a “vector” may comprise any of a variety of nucleic acid molecules into which a desired nucleic acid may be inserted, e.g., by restriction digestion followed by ligation. A vector can be used for transport of such nucleic acid between different environments, e.g., to introduce the nucleic acid into a cell of interest and, optionally, to direct expression in such cell. Vectors are often composed of DNA although RNA vectors are also known. Vectors include, but are not limited to, plasmids and virus genomes or portions thereof. Vectors may contain one or more nucleic acids encoding a marker suitable for use in the identifying and/or selecting cells that have or have not been transformed or transfected with the vector. Markers include, for example, proteins that increase or decrease either resistance or sensitivity to antibiotics or other compounds, enzymes whose activities are detectable by standard assays known in the art (e.g., β-galactosidase or alkaline phosphatase), and proteins or RNAs that detectably affect the phenotype of transformed or transfected cells (e.g., fluorescent proteins). An expression vector is one into which a desired nucleic acid may be inserted such that it is operably linked to regulatory elements (also termed “regulatory sequences”, “expression control elements”, or “expression control sequences”) and may be expressed as an RNA transcript (e.g., an mRNA that can be translated into protein or a noncoding RNA such as an shRNA or miRNA precursor). Regulatory elements may be contained in the vector or may be part of the inserted nucleic acid or inserted prior to or following insertion of the nucleic acid whose expression is desired. As used herein, a nucleic acid and regulatory element(s) are said to be “operably linked” or “operatively associated” when they are positioned so as to place the expression or transcription of the nucleic acid under the influence or control of the regulatory element(s). For example, a promoter region would be operably linked to a nucleic acid if the promoter region were capable of effecting transcription of that nucleic acid. One of skill in the art will be aware that the precise nature of the regulatory sequences needed for gene expression may vary between species or cell types, but can in general include, as necessary, 5′ non-transcribed and/or 5′ untranslated sequences that may be involved with the initiation of transcription and translation respectively, such as a TATA box, cap sequence, CAAT sequence, and the like. Other regulatory elements include IRES sequences. Such 5′ non-transcribed regulatory sequences will include a promoter region that includes a promoter sequence for transcriptional control of the operably linked gene. Regulatory sequences may also include enhancer sequences or upstream activator sequences. Vectors may optionally include 5′ leader or signal sequences. Vectors may optionally include cleavage and/or polyadenylations signals and/or a 3′ untranslated regions. The choice and design of an appropriate vector and regulatory element(s) is within the ability and discretion of one of ordinary skill in the art. For example, one of skill in the art will select an appropriate promoter (or other expression control sequences) for expression in a desired species (e.g., a mammalian species) or cell type. One of skill in the art is aware of regulatable (e.g., inducible or repressible) expression systems such as the Tet system and others that can be regulated by small molecules and the like, as well as tissue-specific and cell type specific regulatory elements. In some embodiments, a virus vector is selected from the group consisting of adenoviruses, adeno-associated viruses, poxviruses including vaccinia viruses and attenuated poxviruses, retroviruses (e.g., lentiviruses), Semliki Forest virus, Sindbis virus, etc. Optionally the virus is replication-defective. In some embodiments a replication-deficient retrovirus (i.e., a virus capable of directing synthesis of one or more desired transcripts, but incapable of manufacturing an infectious particle) is used. Various techniques may be employed for introducing nucleic acid molecules into cells. Such techniques include transfection of nucleic acid molecule-calcium phosphate precipitates, transfection of nucleic acid molecules associated with DEAE, transfection or infection with a virus that contains the nucleic acid molecule of interest, liposome-mediated transfection, nanoparticle-mediated transfection, and the like.

In some embodiments, compositions and methods are used in vivo, e.g., in the treatment of anemia. As known in the art, the term “anemia” refers to a condition characterized by an insufficient number of healthy RBC to transport adequate oxygen to the tissues. See, e.g., Williams Hematology, Goodman & Gilman The Pharmacological Basis of Therapeutics, Basic and Clinical Pharmacology, Harrison's principles of internal medicine, and/or Williams Hematology (all cited above); World Health Organization (2008). Worldwide prevalence of anaemia 1993-2005. Geneva: World Health Organization.

Typically, an individual with anemia has a reduced number of RBCs as compared with the levels present in a normal, healthy individual and/or at least some of the RBCs are abnormal, resulting in reduced RBC function. In certain embodiments, methods of the invention involve administering a HIF-1 activator, a GR activator, or both, to a subject. A “subject”, as used herein, can be a human or a non-human vertebrate in various embodiments. A non-human vertebrate can be, e.g., a mammal, e.g., a non-human primate, a rodent (e.g., mouse, rat, rabbit), or a canine, feline, equine, bovine, ovine, etc. In many embodiments of the invention and in many aspects of the discussion herein, a subject is human. However, in certain aspects the invention envisions applications to treatment of anemia in non-human mammals and applications in research with non-human animals. In some embodiments, a non-human animal serves as a model for a human anemia. In some embodiments, the animal is an immunocompromised animal, e.g., a SCID mouse. In some embodiments, the mouse lacks an endogenous hematopoietic system, e.g., the hematopoietic system has been ablated. In some embodiments, the mouse has a mutation that results in anemia.

One of skill in the art will appreciate that values (e.g., for Hb, hematocrit, etc.), will differ in the case of non-human subjects. In some embodiments, a subject is an adult. In some embodiments a subject is a child (12 years of age or less). In some embodiments a subject (e.g., a human subject) is between 2 and 12 years of age. In some embodiments a subject (e.g., a human subject) is between newborn and 2 years of age. In some embodiments a subject (e.g., a human subject) is a premature infant, e.g., an infant born at less than 36 weeks of gestation, e.g., between 26 and 35 weeks gestation. In some embodiments a subject (e.g., a human subject) is at least 60, 65, 70, 75, or 80 years of age.

Presence of anemia can be determined based on the hemoglobin (Hb) level in the blood. The normal Hb level for adult males is 13 to 18 g/dl; the normal level for non-pregnant adult females is 12 to 16 g/dl. The lower limit of normal is 11.0 g/dl for children 0.5-5.0 years; 11.5 g/dl for children 5-12 years of age; 12.0 for children 12-15 years of age; and 11.5 g/dl for pregnant women. If the Hb level is lower than these values, a subject may be considered to have anemia. In some embodiments, a male subject with anemia has a Hb level of less than 13 g/dl, e.g., less than 12 g/dl, 11 g/dl, 10 g/dl, 9 g/dl, 8 g/dl, 7.5 g/dl, 6.5 g/dl, 6 g/dl, 5.5 g/dl, or 5 g/dl. In some embodiments, a female subject with anemia has a Hb level of less than 12 g/dl, e.g., less than 11 g/dl, 10 g/dl, 9 g/dl, 8 g/dl, 7.5 g/dl, 6.5 g/dl, 6 g/dl. 5.5 g/dl, or 5 g/dl. Anemia can also be defined based on hematocrit, i.e., the proportion, by volume, of the blood that consists of RBCs, expressed as a percentage. Normal values for adult males are 42%-54% and for adult women are 38%-46%. RBC count signifies the number of red blood cells in a volume of blood. Normal range is generally between 4.2 to 5.9 million cells/mm³. Individuals with values below the lower limits for hematocrit and/or RBC count may be considered to have anemia. It will be appreciated that the normal range and lower limits for Hb, hematocrit, and RBC count can vary slightly among different laboratories.

In general, anemia can be caused by a decrease in production of normal red blood cells or hemoglobin or by a loss or destruction of RBCs or RBC precursors. More specifically, anemia can result from excessive blood loss (e.g. acute or chronic bleeding), reduced or ineffective erythropoiesis, or hemolysis (hemolytic anemia, e.g., anemia due to hypersplenism or splenomegaly, autoimmune hemolytic anemia, drug-induced hemolytic anemia, anemia due to mechanical destruction of erythrocytes (e.g. microangiopathic hemolytic anemia, sickle cell anemia), hemolysis due to infections. Anemia can result from renal disease (e.g., chronic renal disease, which can often occur in individuals with diabetes and a variety of other conditions). Renal disease can reduce the kidney's ability to produce Epo, thereby resulting in reduced production of mature RBCs. Although individuals with anemia due to renal disease can often be successfully treated with Epo, some individuals do not exhibit a satisfactory response to Epo, as discussed further below. Anemia can be cancer-associated anemia, which can be a side effect of cancer chemotherapy (e.g., cytotoxic or cytostatic agents). Anemia of chronic disease refers to anemia associated with a chronic infectious, inflammatory, neoplastic disease. Anemia can also be associated with prematurity. Anemia can occur secondary to leukemia or other cancers or conditions that invade the bone marrow. In such diseases, e.g., leukemia, the bone marrow space can become occupied with malignant cells and erythropoiesis can be adversely affected. In these cases erythropoiesis can start to occur outside the bone marrow, representing a form of stress erythropoiesis. In various embodiments, compositions and methods of the invention are of use to treat anemia due to one or more of the afore-mentioned causes, or other causes mentioned herein.

As described herein, HIF activators, e.g., PHIs, in combination with low levels of endogenous or exogenous glucocorticoid receptor agonists stimulate the earliest committed red blood cell progenitors (BFU-E) to increase production of cells that respond to erythropoietin >100-fold. PHIs are known to increase Epo production by the kidney. Based on this mechanism, their use for treating a variety of anemias that can typically be treated using recombinant Epo has been proposed. The finding described herein that PHIs are able to stimulate production of CFU-Es and erythroblasts from BFU-Es opens a new therapeutic window for improved treatment of a large group of anemias that are not treatable with recombinant erythropoietin therapy. Since the effect of PHIs on erythroid progenitors (e.g., BFU-Es) was not demonstrated prior to the findings described herein, the only predicted therapeutic mechanism of PHIs in anemia treatment would involve effects on endogenous erythropoietin production and the subsequent increase in red cell production. Thus, prior to the instant invention, PHIs (and other HIF-1 activators) would not be expected to be useful in treating anemias in which Epo levels are not limiting for RBC production. In addition, prior to the instant invention, the synergistic effect of HIF-1 activators and GR activators was not known.

In various aspects, the invention encompasses any clinical or laboratory use of HIF-1 activators, e.g., PHIs, to increase the numbers of BFU-Es and/or CFU-Es, resulting in an increased number of erythroblasts (rather than merely to replace the effect of rhEpo by stimulating endogenous Epo production). In some embodiments, an anemia is one in which treatment with Epo has limited or no place in standard therapy. In some embodiments, the terms “standard therapy” or “current therapy”, as used herein, refer to therapy that is accepted in the medical and/or surgical field as appropriate for treatment of a particular disease or condition prior to the filing date of the instant application. In some embodiments, the terms “standard therapy” or “current therapy”, as used herein, refer to therapy that is accepted in the medical and/or surgical field as appropriate for treatment of a particular disease or condition prior to the instant invention. In some aspects, “standard therapy” is described in, e.g., Goodman & Gilman The Pharmacological Basis of Therapeutics, Basic and Clinical Pharmacology, Harrison's principles of internal medicine, and/or Williams Hematology (all cited above).

As used herein, “treatment” or “treating” can include amelioration, cure, and/or maintenance of a cure (i.e., the prevention or delay of recurrence) of a disease or condition and/or at least some of its associated symptoms. Treatment after a disorder has started aims, e.g., to reduce, ameliorate or eliminate the disorder, and/or at least some of its associated symptoms, to prevent it from becoming more severe, to slow the rate of progression, or to prevent the disorder or symptoms thereof from recurring. A useful therapy can reduce the severity of the anemia, e.g., restore or maintain an adequate Hb, hematocrit, and/or RBC count, reduce the severity or likelihood of one or more symptoms associated with anemia (e.g., fatigue, shortness of breath, tachycardia). Treatment can be prophylactic, e.g. compounds can be administered to a subject that has not been diagnosed with anemia, e.g., a subject with a significant risk of developing anemia such as a subject expected to undergo surgery that will likely entail considerable blood loss, or a subject expected to undergo chemotherapy. In some embodiments, a therapeutic method of the invention comprises providing a subject in need of treatment for a disease or condition of interest herein. In some embodiments, a therapeutic method of the invention comprises diagnosing a subject in need of treatment for a disease or condition of interest herein. In some aspects, a GR activator is administered for purposes of treating anemia rather than for treating a non-anemia condition for which a GR activator may be indicated. In some embodiments, a subject has elevated average levels and/or elevated peak levels of endogenous glucorticoids (e.g., cortisol) as compared with physiological cortisol levels found, e.g., in a healthy control subject. For example, such levels may be elevated at least about 1.5-fold, 2-fold, 5-fold, 8-fold, or more (e.g., between 2-fold and 10-fold). A control subject can be a subject that is matched with respect to one or more characteristics such as age, sex, etc.

In some embodiments, a method comprises administering an effective amount of a HIF-1 activator, GR activator, or both, to a subject in need thereof. An effective amount can be, e.g., an amount sufficient to achieve a biological response of interest such as an increase in a subject's Hb, hematocrit, or RBC count to a desired level or an increase in a subject's Hb, hematocrit, or RBC count by a desired amount, or a reduction in one or more symptoms of anemia. In some aspects “effective amounts” refers to amounts that are effective when the compounds are administered in combination. In some aspects, “Administered in combination” means that both compounds, i.e., a HIF-1 activator and a GR activator, are administered to a subject sufficiently close in time that they can result in an effect that is greater than if either compound were administered without the other. Such administration is sometimes referred to herein as coadministration. The compounds can be administered in the same composition or separately. When they are coadministered, the two may be given simultaneously or sequentially and in either instance, may be given separately or in the same composition, e.g., a unit dosage (which includes both the HIF-1 activator and the GR activator). In some embodiments, when administered separately, they are administered no more than 1, 2, 4, 8, 12, 24, or 48 hours apart. In some embodiments, they are administered no more than 72, 96, 120, 144, or 168 hours apart. In some embodiments, they are administered no more than 7, 10, or 14 days apart. It will be understood that the compounds are administered to a subject at least once within such time interval and, in some embodiments, multiple times, and such treatment can be continued for, e.g., weeks, months, years, or indefinitely in various embodiments. In some embodiments, administration of two compounds is performed such that (i) a dose of the second compound is administered before more than 90% of the most recently administered dose of the first agent has been metabolized to an inactive form or excreted from the body; or (ii) doses of the first and second compound are administered within 48 hours of each other, or (iii) the agents are administered during overlapping time periods; or (iv) any combination of the foregoing. The HIF-1 activator can be given prior to or after administration of the GR activator, provided that they are given sufficiently close in time to have a desired effect. One or more than one HIF-1 activator and one or more than one GR activator can be administered according to the present method.

In some aspects, a subject treated using a method of the instant invention suffers from or is at risk of an Epo-resistant anemia. As used herein, “Epo-resistant anemia” refers to an anemia in which administration of Epo does not result in a desired response and/or in which administration of Epo would not be expected to result in a desired response. In some aspects, an anemia in which treatment with Epo has limited or no place in standard therapy is considered an Epo-resistant anemia. In some aspects, a subject suffering from or at risk of an anemia in which Epo has limited or no place in standard therapy is considered to be suffering from an Epo-resistant anemia. It should be noted that a number of erythropoiesis stimulating agents (ESAs) related to Epo are known. For example, such agents can be Epo variants having an amino acid sequence that differs from that of human Epo at one or more positions (e.g., between 1 and 5 positions) and/or having a different glycosylation pattern (e.g., additional oligosaccharide chains). One example is darbepoietin alfa. In general such agents act in a similar manner to Epo although different doses and dosing regimens are often applicable. Therefore, an Epo-resistant anemia will typically be resistant to these ESAs as well.

As known in the art, some individuals suffering from an anemia of a type that frequently responds to administration of Epo do not experience a desired response to Epo. Such individuals may be considered to be suffering from an Epo-resistant anemia. For example, around 90% of subjects with renal disease-associated anemia treated with rhEpo respond with a significant rise in Hb concentration. The rest exhibit a suboptimal response, either failing to reach target Hb levels despite high doses of rhEpo, or by the loss of a previously satisfactory response (see, e.g., Macdougall I C and Cooper A C. Erythropoietin resistance: the role of inflammation and pro-inflammatory cytokines. Nephrol Dial Transplant. 2002; 17 Suppl 11:39-43, and references therein). Thus up to about 10% of patients with anemia due to renal disease who receive recombinant human erythropoietin therapy show poor responsiveness to the drug and are considered to have Epo-resistant anemia.

Whether a subject is or is not suffering from an Epo-resistant anemia can be determined by assessing the subject's response or predicted response to treatment with recombinant human Epo. For example, in some embodiments, a subject may be deemed to have an Epo-resistant anemia if the subject displays an increase in hemoglobin concentration of less than 2 g/dl, or fails to reach levels of at least 12 g/dl, after undergoing a standard regimen of treatment with recombinant human EPO. In some embodiments, a subject may be deemed to have an Epo-resistant anemia if the subject fails to reach or maintain a hemoglobin concentration at least 10 g/dL. In some embodiments, a desired response to treatment with recombinant Epo can be defined as an increase in hemoglobin of at least 2 g/dl over a twelve (12) week dosing regimen. If a subject does not display such a response within such time period, the subject is considered resistant to treatment with recombinant human Epo, i.e., the subject suffers from an Epo-resistant anemia. In some embodiments of the invention, a subject can be considered resistant to treatment with rhEpo if treatment with rhEpo according to a particular dosing regimen fails to increase the subject's hemoglobin level by at least between 0.1-5.0 g/dL. In some embodiments, a subject is resistant to treatment with rhEpo if such treatment fails to increase the subject's hemoglobin level by at least 0.2, 0.5, 1.0, 1.5, 2.0, 2.5, 3.0, 3.5, 4.0, 4.5, or 5.0 g/dL. In some embodiments, a target hemoglobin level for an adult subject receiving rhEpo is 12 gm/dL. Therefore, in some embodiments, a subject is considered resistant to rhEpo therapy if treatment with acceptable doses over a specific period of time fails to increase hemoglobin to at least 12 gm/dL. In other embodiments, a subject is a subject resistant to rhEpo therapy if treatment with acceptable doses over a specific period of time fails to increase hemoglobin to at least 10 gm/dL, or at least 11 gm/dL. In some embodiments, a subject may be considered to have an Epo-resistant anemia if the subject fails to reach a hematocrit of at least 36% after undergoing a standard regimen of treatment with recombinant human Epo. In various embodiments, the subject is resistant to treatment with rhEpo if a standard dosing regimen fails to raise the subject's hematocrit to at least 30%, at least 32%, at least 34%, at least 36%, or at least 38%. It will be understood that the criteria for determining whether a subject suffers from an Epo-resistant anemia, e.g., the value of the desired increase in hemoglobin or hematocrit, may vary depending on a number of factors, including age, sex, etc. One of skill in the art will be aware of suitable methods for determining that a subject suffers or is at risk of suffering from an Epo-resistant anemia.

Non-limiting discussion of certain anemias is presented in the following paragraphs. It should be noted that certain agents or disease can give rise to different types of anemias, and certain individuals have anemia due to multiple causes. The discussion and classification below is not intended to limit the invention. Various aspects of the invention encompass use of a HIF-1 activator (e.g., a PHI), optionally in combination with a GR activator, to treat a subject suffering from or at risk of any of these anemias.

1. Bone Marrow Failure Syndromes (see, e.g., Elebute et al., 2003; Erslev, 2000; Gaman et al., 2009 Ru, 2009; Spivak et al., 2009).

Aplastic Anemia (AA)

Aplastic anemia (AA) is defined as a peripheral blood pancytopenia with a hypocellular bone marrow (Gaman et al., 2009). Secondary AA can be caused by radiation, various drugs or other chemicals, viruses (Epstein Barr, hepatitits virus, parvovirus, human immunodeficiency virus), parasites (e.g., malaria), immune diseases (eosinophilic fasciitis, thymoma), paroxysmal nocturnal hemoglobinuria, pregnancy; other cases of acquired aplastic anemia are represented by idiopathic aplastic anemia.

Most forms of AA are not currently treated with erythropoietin. The results described herein suggest that HIF-1 activators, e.g., PHIs, can be of significant benefit in these conditions since they act by enhancing BFU-E self-renewal and will increase production of the cells that are lacking in these individuals.

The invention, in various aspects, proposes use of HIF-1 activators, e.g., PHIs, optionally in combination of anti-thymocyte, cyclosporine A and/or glucocorticoids or other GR agonists, to treat acquired AA, optionally in further combination with transfusions, antibiotics, androgens and hematopoietic growth factors, in treatment of AA.

Congenital (e.g. Fanconi, Schwachman-Diamond, and Dyskeratosis congenita).

Congenital forms of AA all have a component of erythroid progenitor deficiency that causes anemia. These conditions would benefit from a drug that enhances formation of erythroid progenitors. The disclosure herein shows for the first time show that PHIs are able to directly stimulate BFU-E progenitor self-renewal and CFU-E generation. This means PHIs are excellent candidate drugs for treatment of congenital AA. Currently, certain of these anemias are treated with GCs or androgens as standard therapy. It is anticipated that PHIs will reduce required doses of glucocorticoids or androgens and thus reduce harmful side effects of these drugs.

Acquired (e.g. radiation, chemo therapy, paroxysmal nocturnal hemoglobinuria (PNH), autoimmune disease, malignancy)

Paroxysmal nocturnal haemoglobinuria (PNH) is an acquired clonal disorder of the haemopoietic stem cell (HSC). Glycosylphosphatidylinositol-deficiency leads to hypercellular bone marrows with erythroid hyperplasia, normal blood counts or mild peripheral blood cytopenias (Elebute et al., 2003). The invention encompasses administering a HIF-1 activator, e.g., a PHI, optionally in combination with a GR activator, e.g., a GC such as prednisone, to a subject suffering from or at risk of PNH.

2. Pure Red Cell Aplasia

The findings described herein suggest that any anemia involving red cell aplasia (e.g., associated with insufficient numbers of Epo-sensitive progenitors in the bone marrow) has the potential to be treated by HIF-1 activators, e.g., PHIs. Pure red cell aplasia (PRCA) is a syndrome characterized by a severe normocytic anaemia, reticulocytopenia, and absence of erythroblasts from an otherwise normal bone marrow. Primary PRCA, or secondary PRCA which has not responded to treatment of the underlying disease, is treated as an immunologically-mediated disease. Corticosteroids are considered the agents of first choice in standard treatment of many forms of PRCA. Although the therapeutic effect of GCs in PRCA in part is attributed to the anti-inflammatory effect, without wishing to be bound by theory, a direct stimulatory effect on BFU-E progenitors likely contributes. The instant invention encompasses administering a HIF-1 activator, e.g., a PHI, optionally in combination with a GR activator, e.g., a GC, to a subject with PRCA. In some embodiments, the use of the HIF-1 activator allows a reduced dose of the GC.

Congenital PRCA

Diamond Blackfan Anemia (DBA) is an example of a form of anemia that does not respond to Epo treatment. In accordance with the instant invention, DBA can be treated using a HIF-1 activator, e.g., a PHI. The anemia in DBA patients is often successfully treated with glucocorticoids or androgens (Flygare and Karlsson, 2007). It is anticipated that use of HIF-1 activators, e.g., PHIs, will reduce required doses of glucocorticoids or androgens and thus reduce harmful side effects of these drugs.

Acquired PRCA (e.g. induced by virus, drugs or other chemicals, malignancies, myelodysplastic syndromes, autoimmune disease, parasites (e.g. malaria) and transient erythroblastopenia of childhood (TEC).

Different levels of pure red cell aplasia is commonly associated with virus infection (e.g., HIV, HBV, EBV), malignancy, inflammation and autoimmune disease. These conditions often do not respond well to Epo treatment and would not be expected to benefit from further from PHI treatment based on previous knowledge. The findings herein suggest that the effect of PHIs on BFU-E cells would be very beneficial for any form of pure red cell In certain embodiments of the invention, PHIs can be used to stimulate BFU-E cells in these conditions to increase the pool of Epo-responsive CFU-E cells. This will lead to increased production of RBCs.

Anemia associated with malaria (caused by various Plasmodium species) involves direct and indirect destruction of erythroid progenitors (Ru et al., 2009). We propose use of PHIs to enhance erythroid progenitor production in anemic malaria patients. Such therapy can be used in combination with any anti-Plasmodium therapy.

Anemia associated with myelodysplastic syndromes (MDS) is associated with ineffective erythropoiesis, manifested as hypercellular bone marrow with peripheral cytopenias. Anemia associated with 5q MDS is considered to be an acquired form of DBA-like anemia. Anemia in 10-30% of MDS patients can be ameliorated by recombinant Epo treatment. The group of anemic MDS patients that do not respond to Epo would not be expected to respond to PHI treatment based on previous knowledge. The findings described herein that PHIs enhance CFU-E regeneration however suggest that PHIs (or other HIF-1 activators) are excellent candidates to treat anemic MDS patients.

3. Hemolytic Anemia (Use PHIs to Increase the Pool of CFU-E and Erythroblasts in Order to Enhance the Rate of RBC Production)

Although patients with hemolytic anemia do not typically present with reduced numbers of CFU-Es in the bone marrow, it is expected that a PHI-induced increase of CFU-E progenitors would be beneficial for many of these patients. The increased production of Epo-responsive CFU-E progenitors would increase the red blood cell output.

4. Anemia Associated with Ageing

Idiopathic anemia in older individuals is associated with reduced cytokine response and erythroid progenitor deficiency. Importantly BFU-E progenitors in older individuals produce fewer CFU-Es in response to glucocorticoids than younger individuals (Morra et al., 1993). We propose use of PHIs to increase CFU-E regeneration from BFU-E progenitors in elderly people. Normalizing hemoglobin values in this population will likely have significant beneficial effects on cognitive function and quality of life. In some embodiments, such treatment is administered to an individual at least 65, 70, 75, or 80 years of age.

5. Blood Loss.

Anticipated

Increased readiness of the hematopoietic system to recover from expected blood loss would be of significant benefit. The invention encompasses the use of PHIs (or other HIF-1 activators) to increase the CFU-E numbers before, during and/or after situations where severe blood loss is expected or occurs (e.g. surgeries frequently requiring blood transfusions). It is anticipated that PHIs can be used to preoperatively increase CFU-E numbers in the bone marrow. After blood loss, endogenous Epo levels will rise and stimulate CFU-E cells in the bone marrow (or an ESA can be administered). By preoperatively using PHIs to form a larger pool of CFU-E progenitors, normalization of hemoglobin values will occur faster and the need for peri and postoperative blood transfusions will be reduced. Of course PHIs (or other HIF-1 activators), optionally in combination with a GR activator, can be administered after surgery or other cause of significant blood loss such as severe injury, childbirth, blood vessel rupture, to increase BFU-E self-renewal and thereby increase CFU-E generation, resulting in increased numbers of RBCs.

Chronic

Patients with chronic blood loss (e.g., blood loss continuing over a period of at least 4 weeks, often at least 8, 12, 20, or more weeks) will often, like patients with hemolytic anemia, have elevated Epo levels and continuously produce new red blood cells from CFU-Es. In some embodiments, the invention encompasses use PHIs (or other HIF-1 activators) to promote CFU-E regeneration in these conditions in order to reduce the use of transfusions and speed up recovery.

6. Epo-Resistant Subjects with from Anemia of a Type that Typically Responds to Epo

As noted above, anemia associated with chronic renal disease or cancer chemotherapy can often be successfully treated with rhEpo (or related molecules). However, not all subjects respond. In certain embodiments, the invention provides methods of treating a subject suffering from such an anemia comprising administering a HIF-1 activator to the subject. Optionally the method further comprises administering a GR agonist, e.g., a GC, to the subject. In some embodiments, rhEpo is administered as well. In some embodiments, a subject is on dialysis. In other embodiments, a subject is not on dialysis.

7. Anemia is Associated with Sepsis

Sepsis is a serious medical condition characterized by a whole-body inflammatory state and the presence of a known or suspected infection. This inflammatory state may develop in response to microbes (e.g., bacteria, fungi) in the blood, urine, lungs, skin, or other tissues. In some embodiments of the invention a HIF-1a activator and GR activator are administered to a subject at risk of or suffering from anemia associated with sepsis.

The therapeutic methods of the invention may be used together with one or more additional pharmacological therapies or non-pharmacological therapies, or combinations thereof, for treating a subject. One of skill in the art can select an appropriate additional therap(ies) based, e.g., on the particular disease. For example, a subject suffering from anemia due to an infectious agent can be treated with an appropriate anti-infective agent. A subject suffering from cancer can be treated using cancer chemotherapy appropriate for the particular cancer, surgery, etc.

In various methods of the invention, compounds can be used or administered in a single dose or multiple doses, e.g., 1, 2, 3, or more times a day, every other day, weekly, bi-weekly, or monthly. The dose administered can depend on multiple factors, including the identity of the compound, weight of the subject, frequency of administration, etc. In some aspects, a subject will receive one or more courses of therapy, each involving administration of multiple doses. Doses of compounds may range from about 1 μg to 10,000 mg, e.g., about 10 μg to 5000 mg, e.g., from about 100 μg to 1000 mg once or more per day, week, month, or other time interval, in various embodiments of the invention. Stated in terms of subject body weight, doses in certain embodiments of the invention range from about 1 μg/kg/day to 20 mg/kg/day, e.g., from about 0.1 mg/kg/day to 10 mg/kg/day. In certain embodiments doses are expressed in terms of surface area rather than weight, e.g., between about 1 mg/m² to about 5,000 mg/m². The absolute amount will depend upon a variety of factors including the concurrent treatment, the number of doses and the individual patient parameters including age, physical condition, size and weight. These are factors well known to those of ordinary skill in the art and can be addressed with no more than routine experimentation. Exemplary doses may be selected using in vitro studies, tested in animal models, and/or in human clinical trials as standard in the art. Toxicity and therapeutic efficacy of molecules can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD50/ED50. In some aspects, compounds that exhibit high therapeutic indices are used. In the case of compounds that have been tested already in preclinical studies and/or clinical trials, considerable information is already available that can be used in selecting doses. In some aspects, a compound, e.g., a GC, is already in use as a therapeutic agent. Suitable doses can be estimated based on doses that are considered medically acceptable. In some aspects, a dose of a GC is lower than those that are typically used in common indications for GC administration, e.g., conditions that have an inflammatory or immune-mediated component such as arthritis, colitis, asthma, allergies, transplant rejection, etc. In some embodiments, a dose is selected to achieve a GC level equivalent to between a 2-10 fold increase in a normal physiological cortisol level.

In certain embodiments each of the compounds is administered in an amount that is effective when used as a single agent. In certain embodiments at least one of the compounds is administered in an amount that would be sub-therapeutic or less than optimally therapeutic if the compound were administered as a single agent. In certain embodiments at least one of the compounds is administered in an amount that is lower than the maximum tolerated dose, e.g., the compound is administered in an amount that is about 5%, 10%, 20%, 30%, 40%, 50%, 60%, 70%, 80%, or 90% of the effective amount or maximum tolerated dose. For example, in some embodiments of the invention a HIF-1 activator is coadministered with a GR activator to treat anemia, wherein the amount of the HIF-1 activator used is lower than that required to have an equivalent therapeutic effect in the absence of the GR activator. In some embodiments of the invention a GR activator is coadministered with a HIF-1 activator to treat anemia, wherein the amount of the GR activator used is lower than that required to have an equivalent therapeutic effect in the absence of the HIF-1 activator. In some aspects, e.g., in the case of an anemia for which a GC is standard therapy (e.g., Diamond-Blackfan anemia), administration of a HIF-1 activator allows use of a reduced dose of the GC. In some aspects, e.g., in the case of an anemia for which use of a HIF-1 activator is proposed, administration of a GR activator in combination allows use of a reduced dose of the HIF-1 activator and/or results in enhanced efficacy.

A compound can be provided in a pharmaceutical composition, which comprises the compound and one or more pharmaceutically acceptable carrier(s). Pharmaceutically acceptable carriers are well known in the art and include, for example, aqueous solutions such as water, 5% dextrose, or physiologically buffered saline or other solvents or vehicles such as glycols, glycerol, oils such as olive oil or injectable organic esters that are suitable for administration to a human or non-human subject. In some embodiments, a pharmaceutically acceptable carrier or composition is sterile. A pharmaceutical composition can comprise, in addition to the active agent(s), physiologically acceptable compounds that act, for example, as bulking agents, fillets, solubilizers, stabilizers, osmotic agents, uptake enhancers, etc. Physiologically acceptable compounds include, for example, carbohydrates, such as glucose, sucrose, lactose; dextrans; polyols such as mannitol; antioxidants, such as ascorbic acid or glutathione; preservatives; chelating agents; buffers; or other stabilizers or excipients. The choice of a pharmaceutically acceptable carrier(s) and/or physiologically acceptable compound(s) can depend for example, on the nature of the active agent, e.g., solubility, compatibility (meaning that the substances can be present together in the composition without interacting in a manner that would substantially reduce the pharmaceutical efficacy of the pharmaceutical composition under ordinary use situations) and/or route of administration of the composition. Compounds can be present as salts in a composition. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts. It will also be understood that a compound can be provided as a pharmaceutically acceptable pro-drug, or an active metabolite can be used. Furthermore it will be appreciated that agents may be modified, e.g., with targeting moieties, moieties that increase their uptake, biological half-life (e.g., pegylation), etc. It will be understood that compounds can exist in a variety or protonation states and can have a variety of configurations and may exist as solvates (e.g., with water (i.e. hydrates) or common solvents) or different crystalline forms (e.g., polymorphs). The invention is intended to encompass embodiments exhibiting such alternative protonation states, configurations, and forms.

The pharmaceutical composition could be in the form of a liquid, gel, lotion, tablet, capsule, ointment, transdermal patch, etc. A pharmaceutical composition can be administered to a subject by various routes including, for example, parenteral administration. Exemplary routes of administration include intravenous administration; respiratory administration (e.g., by inhalation), nasal administration, intraperitoneal administration, oral administration, subcutaneous administration, intrasynovial administration, transdermal administration, and topical administration. For oral administration, the compounds can be formulated with pharmaceutically acceptable carriers as tablets, pills, dragees, capsules, liquids, gels, syrups, slurries, suspensions, etc. In some embodiments at least one of the compounds is administered by release from an implanted sustained release implant or device. A sustained release implant could be implanted at any suitable site. In some embodiments, a sustained release implant delivers therapeutic levels of the active agent for at least 30 days, e.g., at least 60 days, e.g., up to 3 months, 6 months, or more. When multiple compound(s) are administered, they can be administered by the same or different routes in various embodiments. In some embodiments, the HIF-1 activator and GR activator are administered orally. In some embodiments, the invention relates to an oral dosage form that contains a HIF-1 activator (e.g., a PHI) and a GR agonist, e.g., a GC, e.g., prednisone. In some embodiments, the amounts of the compounds are selected to provide a synergistic beneficial effect in treating anemia.

In some embodiments, a compound is delivered by means of a microparticle or nanoparticle or a liposome or other delivery vehicle or matrix. A number of biocompatible synthetic or naturally occurring polymeric materials are known in the art to be of use for drug delivery purposes. Examples include polylactide-co-glycolide, polycaprolactone, polyanhydride, cellulose derivatives, and copolymers or blends thereof. Liposomes, for example, which consist of phospholipids or other lipids, are nontoxic, physiologically acceptable and metabolizable carriers that are relatively simple to make and administer.

### EXAMPLES

### Overview of the Examples

In an effort to identify compounds with potential to enhance CFU-E regeneration, we chose to study the physiological mechanism by which Epo-responsive progenitors are replenished during severe or chronic anemia. This process, which in vivo involves stem cell factor (SCF) and glucocorticoids (GCs), is often referred to as stress erythropoiesis (SE) (Bauer et al., 1999; Kolbus et al., 2003; Wessely et al., 1997). SE can be replicated and studied in vitro, by culturing erythroid progenitors in medium containing SCF, Dexamethasone (Dex) and Epo (Bauer et al., 1999; Kolbus et al., 2003; Wessely et al., 1997). Both in vitro proliferation of fetal liver erythroblasts and SE in vivo are severely decreased by a mutation in the glucocorticoid receptor (GCR) that disrupts dimerization and transactivation but not transrepression (Bauer et al., 1999; Reichardt et al., 1998). Since deletion of the GCR AF2 transactivation domain also disrupts erythroid proliferation (Wessely et al., 1997), it is believed that GCs stimulate erythroblast regeneration during SE by gene activation rather than repression (Bauer et al., 1999; Reichardt et al., 1998).

Studies of how GCs stimulate SE have been limited by the fact that the cell type(s) that responds to GCs has not heretofore been identified. For instance, the observed increase in erythroblast numbers during SE could in theory arise from enhanced self-renewal of erythroblasts or of earlier progenitor cells such as CFU-E or BFU-E or progenitors thereof. As described further herein, cultured CFU-E and BFU-E cells, purified from mouse fetal liver by a new technique, were used to demonstrate that BFU-E cells respond to GCs by generating more daughter BFU-E cells. As a consequence over time this increases the number of CFU-E cells and thus the eventual number of differentiated erythroid cells formed from each BFU-E. As discussed below, we showed that GCs have no significant intrinsic effect on CFU-E proliferation, thus confirming that GCs enhance erythroblast generation during SE by acting on BFU-Es to increase their proliferation.

By next generation mRNA sequencing we identified genes in BFU-E cells that are upregulated >2-fold by GC treatment. Computational analyses indicate that the majority of the promoters of these upregulated genes are predicted to bind the transcription factors myelomastosis oncogene (MYC) and hypoxia-induced factor 1 alpha (HIF1a), but interestingly not GCR, suggesting that GCR actives SE through or in cooperation with MYC and/or HIF1a activation. Indeed, treatment of BFU-Es with glucocorticoids together with dimethyloxalylglycine (DMOG), a prolyl hydroxylase inhibitor (PHI) and thus a stabilizer of HIF-1 alpha, leads to enhanced BFU-E self-renewal and an ˜300-fold total increase in production of erythroblasts from each BFU-E.

Without wishing to be bound by any theory, we propose a physiological model of stress erythropoiesis where increased systemic levels of free cortisol, in combination with local anoxia, increase self-renewal of BFU-E erythroid progenitors and thus CFU-E output, promoting a rapid and long-lasting increase in red blood cell production. Importantly, we show that the mechanism of CFU-E regeneration during SE can be pharmacologically stimulated by PHIs in combination with low GC concentrations. These results demonstrate, among other things, the clinical potential of PHIs to be used to increase the response to Epo by increasing the number of CFU-E progenitors in Epo-resistant anemias and for treatment of bone marrow failure syndromes that manifest CFU-E deficiency.

### Example 1

### Purification of BFU-E and CFU-E Cells from Fetal Liver by Flow Cytometry

We developed techniques to purify BFU-E and CFU-E cells from mouse fetal liver by magnetic depletion and flow cytometry. A purified fraction of BFU-E and CFU-E cells was obtained by negative selection for Ter119, B220, Mac-1, CD3, Gr-1, Sca-1, CD16/32, CD41 and CD34, and positive selection for c-kit (See below and Table S1). In our initial studies BFU-E cells were then separated from CFU-E cells based on the relatively low level of CD71 or CD24a expression in BFU-E cells compared to CFU-E cells (See below and Table S2). We scored BFU-E colonies as late (small)-BFU-E-derived colonies (i.e., colonies derived from late BFU-E cells) if they consisted of 5-20 clusters, or as early (large)—BFU-E-derived colonies (i.e., colonies derived from early BFU-E cells) if they consisted of more than 20 clusters (FIG. S1). Since some BFU-E cells formed clusters of CFU-E colonies already on day 3, only cells forming a single CFU-E colony were scored as a CFU-E cell.

An efficient BFU-E and CFU-E cell separation was achieved using CD71 (FIG. S2 and Table S2). Among the sorted CD71^{20% high} (CFU-E fraction) cells, 61% formed single-CFU-E colonies in the absence of Dex and 57% with Dex. Cells in this CFU-E fraction formed no early BFU-E-derived colonies, and only 0.2% formed late BFU-E-derived colonies when cultured in the absence of Dex; in the presence of Dex these percentages were 0% and 1.7%, respectively (Table S2).

In the absence of Dex, 11% of purified CD71^{10% low} (BFU-E) cells formed CFU-E colonies (6.6% with Dex). In the absence of Dex, 19% formed late BFU-E colonies (29% with Dex), 6.6% formed early BFU-Es (22% with Dex), and 2.1% formed CFU-G/M/Meg colonies (2.0% with Dex) (Table S2; see also Table 1, 0 hr culture). No GEMM colonies were formed (Table S2).

In later studies we purified BFU-E and CFU-E cells based on low or high expression, respectively, of both CD71 and CD24a, and obtained slightly higher purity of BFU-E cells with a higher percentage of early-BFU-Es (Table S2). The efficient separation of BFU-E and CFU-E allowed us for the first time to determine which cell GCs intrinsically stimulate and the nature of the resultant physiological and molecular effects.

Further Details and Discussion of Example 1

We chose to purify erythroid progenitors from mouse E14.5-15.5 fetal liver (FL) rather than bone marrow (BM) since the concentration of these cells is higher in FL, and since ˜90% of these cells are erythroid. Although peripheral blood contains BFU-E cells, it was not used since very few CFU-E cells are found in the circulation (Praloran et al., 1989). We assayed levels of enrichment of BFU-E and CFU-E cells by colony forming assays (FIG. S1); as detailed later, some of these colony-forming assays were performed with and without 100 nM Dex in order to simultaneously determine which colony-forming cells are stimulated by GCs.

The first step towards enrichment of BFU-E and CFU-E cells was to remove more mature blood cells from single cell suspensions of fetal livers by magnetic column depletion. We stained fetal liver cells with a panel of biotin labeled anti-lineage (Lin) antibodies (Ter119, B220, Mac-1, CD3 and Gr-1), followed by anti-biotin tetramer and magnetic colloid labeling. All positive cells were depleted using magnetic columns. As anticipated from previous studies the Lin-fraction contains mostly CFU-Es and BFU-Es, but also non-erythroid (CFU-G/M/Mk) and multi-potent (GEMM) colony forming cells (Table S1) (McKearn et al., 1985). As suggested by previous studies on mouse BM progenitor cells (Pronk et al., 2007; Terszowski et al., 2005), further depletion using Sca-1 removed early multipotent GEMM colony forming cells, and reaction with CD16/32 (FcgR), CD41 and CD34 antibodies depleted myeloid colony-forming cells (CFU-G/M/Mk). The number of erythroid colony-forming cells was unaffected (Table S1), resulting in a very pure fetal liver erythroid progenitor (FLEP) cell population. Although only 50-70% of FLEP cells formed colonies, 99% of colonies that did form were BFU-Es or CFU-Es (Table S1). Thus FLEP cells are a mix of BFU-E and CFU-E cells with possible contamination of dead, apoptotic, or other non-colony-forming cells.

In order to further purify and separate populations of BFU-E and CFU-E cells from FLEP cells, we used flow cytometry (FIG. S2). Since BFU-E and CFU-E cells both express c-kit (Terszowski et al., 2005), and since CFU-E cells in BM express high levels of the transferrin receptor (CD71) (Terszowski et al., 2005) and mature erythroid cells high levels of CD24a (Nielsen et al., 1997), we hypothesized that the level of CD71 or CD24a expression could be used to separate CFU-E and BFU-E cells from c-kit+ FLEP cells.

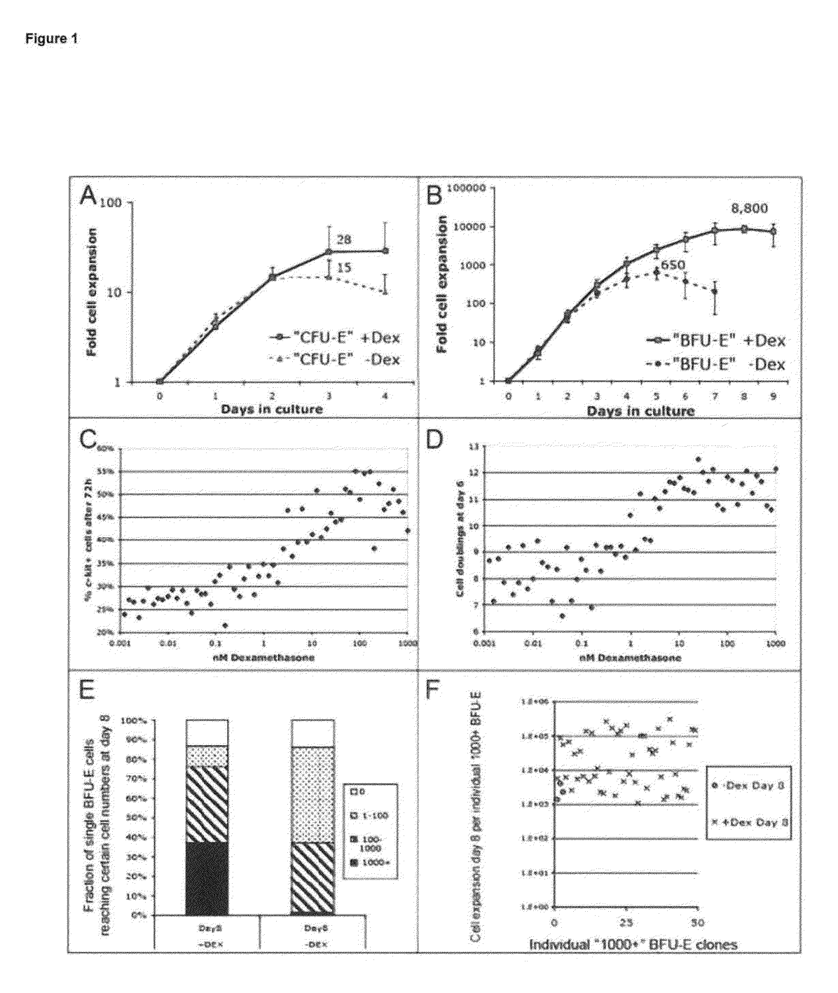

Based on the fact that ≈15% of FLEP cells formed BFU-E colonies, we sorted the fractions of the 10% lowest CD71 (or CD24a) expressing c-kit+ FLEP cells. Similarly, since ≈60% of FLEP cells formed CFU-E colonies we hoped to obtain pure single-CFU-E forming cells by sorting the 20% highest CD71 (or CD24a) expressing c-kit+ FLEP cells. Indeed, Table S2 shows that this is the case. As judged by colony-forming assays BFU-E and CFU-E-forming cells could be separated to high purities using either antibodies against CD71 or CD24 antigen, with a slightly lower purity using CD24a than CD71 (Table S2). The high purity of BFU-Es enriched by CD71 is further shown in the single cell culture experiments (FIG. 1E), which demonstrate that in presence of Dex more than 75% of BFU-E cells generate more than 100 erythroblasts (while more than 10% do not survive the sorting procedure) (FIG. 1E). Since a CFU-E will not divide more than 6 times (and can not generate 100 erythroblasts) more than 75% of the cells in this sorted population are earlier progenitors than CFU-E cells.

In methyl-cellulose assays the different 10%^{low} and 20%^{high} sorted cell populations demonstrated a total colony-forming efficiency (sum of all colonies formed divided by 1000) ranging from 40-70%. The fact that 100% of cells do not form colonies could be explained by contamination of viable but non-colony-forming cells, or more likely by decreased cell viability caused by the extensive cell manipulation involved in fetal liver harvest, magnetic depletion, cell sorting, and the colony-forming assay procedure. The latter explanation is supported by the single cell culture experiments (FIG. 1E) and the homogenous morphology of cells within each sorted cell population (FIG. S3). The purities of BFU-E and CFU-E populations can therefore be estimated as the ratio of the number of BFU-E or CFU-E colonies to the total number of colonies formed in the presence of Dex. The BFU-E purity would then be 86% using CD71 only and 94% using both CD71 and CD24a (37% and 59% respectively of total cells being early (large) BFU-E-forming cells). Importantly, this highly enriched BFU-E population contains <1% CFU-G/M/Mk and no detectable CFU-GEMM cells. To our knowledge these populations represent the purest separation and enrichment for BFU-E and CFU-E cells ever described, although there are several reports describing efforts to isolate CFU-E (Nijhof and Wierenga, 1983; Terszowski et al., 2005) and BFU-E cells (Heath et al., 1976; Sawada et al., 1990).

### Example 2

### GCs Stimulate CFU-E Regeneration by Allowing More BFU-E Cell Divisions to Occur Prior to Differentiation into CFU-Es

The proliferative potential of CFU-E and BFU-E cells was determined by liquid culture in serum-free erythroid liquid expansion (SFELE) medium (see Experimental Procedures) with and without 100 nM Dex. Addition of Dex increased the average number of erythroblasts formed from each CFU-E cell from 15 to 28 (FIG. 1A). In contrast, the proliferative capacity of “BFU-E” cells was dramatically increased by addition of Dex, allowing each BFU-E cell to divide on average 13 instead of 8 times, corresponding to an increase in cell number from 650 to 8,800 cells per original BFU-E cell (FIG. 1B). For the first 3 days the doubling time of sorted BFU-E cells was unaffected by Dex. At the peak of BFU-E cell expansion (day 5 without Dex, and day 8 with Dex), almost all nucleated cells were mature erythroblasts by morphology, and thus the product of terminal CFU-E proliferation and differentiation (data not shown). These cell proliferation-experiments were repeated using CD24a^{20% high} CFU-E cells and CD24a^{10% low} BFU-E cells; similar results were obtained (data not shown).

We suggest that although Dex slightly increases proliferation of “CFU-E” cells, this is not due to self-renewal of CFU-E cells or erythroblasts but rather due to contamination of small numbers of multi CFU-E and late BFU-E cells within the purified CFU-E population (Supplemental Table S2). Instead, our findings suggest that GCs act on BFU-E cells by maintaining the immaturity of BFU-Es. This is supported by FACS analysis of “BFU-E” cells at day 3 of culture, which shows that 55% still expressed the early progenitor marker c-kit when cultured with 100 nM Dex, compared to only 26% in cultures without Dex (FIG. 1C). Both Panels C and D in FIG. 1 demonstrate that Dex concentrations below 1 nM have little stimulatory effect on BFU-E proliferation while 10 nM Dex supports maximal stimulation.

In order to establish that Dex indeed maintains BFU-E immaturity during successive cell divisions, sorted BFU-E cells were cultured in SFELE medium with and without Dex and then subjected to colony forming assays (Table 1). When cultured in the presence of Dex each initial sorted CD71^{10% low} BFU-E cell on average gives rise to 67 late BFU-Es by Day 2, and this number increases further to 153 late BFU-E's by Day 3. In sharp contrast, in the absence of Dex each initial BFU-E cell gives rise to only 30 late BFU-Es by Day 2, and the number of late BFU-Es in the culture decreases to 11 by Day 3. Thus the presence of Dex increases the self-renewal capability of BFU-E-s such that after each division—requiring only 12 hours in this culture system (FIG. 1)—the probability of a daughter cell remaining a BFU-E is higher in the presence of Dex than in its absence.

Whether or not Dex is added to the cultures, the number of CFU-Es increases at about the same rate for the first three days: by Day 3, 74 CFU-Es are formed per BFU-E in the absence of Dex and 122 in its presence. Strikingly, the number of CFU-Es in the culture without Dex drops sharply between Day 3 and Day 5. That is because no further CFU-Es are formed after 3 days, and by Day 5 the CFU-E cells present at 3 days have differentiated into mature erythroblasts and thus have lost their colony-forming potential. In marked contrast, in cultures with Dex the number of CFU-E cells further increases 7.3 fold between Days 3 and 5. This attests to continued formation of CFU-E cells from the earlier BFU-E cells between Days 3 and 5, consistent with the much higher numbers of BFU-E cells in the Dex-containing cultures at Day 3 than in cultures without Dex.

Thus this experiment establishes that Dex increases the probability that a given BFU-E will divide and form daughter BFU-E cells (“self-renewal”) and reduces the probability that a given late BFU-E cell will form a CFU-E (“differentiate”) during each cell division. This agrees with colony-forming assays showing that Dex increases both the size and number of BFU-E colonies (FIG. S1). In other words, GCs act by maintaining progenitor immaturity during cell division of BFU-E daughter cells, allowing over time increased numbers of divisions of individual BFU-E cells and thus increased numbers of CFU-E cells to be formed from each BFU-E.

### Example 3

### Earlier Cells in the BFU-E to CFU-E Continuum have Greater Potential to be Stimulated by GCs

The “BFU-E” cell population that contains the GC-responsive cells, while highly enriched for BFU-Es, is a heterogeneous mix of erythroid progenitors. In order to determine the effect of GCs at the single cell level, we performed single “BFU-E” cell culture experiments (FIG. 1E). By Day 8 of culture in the presence of Dex>75% of sorted CD71^{10% low} BFU-E cells had formed more than 100 cells in erythroid expansion culture (a CFU-E will not form more than 64), indicating that the purity of these BFU-E cell populations is likely greater than that estimated from colony-forming assays. Importantly, 37% of BFU-E cells had expanded more than 1000-fold in response to Dex, while only 1.7% did so without Dex. While some BFU-E cells performed more than 18 cell divisions in 8 days in response to Dex the maximum number of cell divisions were 12 without Dex. Thus, at the single cell level, 100 nM Dex allows more than 6 additional cell divisions to occur (FIG. 1F) and Dex has a proportionally greater stimulatory effect on earlier than later BFU-E progenitors.

### Example 4

### Glucocorticoids Regulate Transcription of a Small Number of mRNAs but not miRNAs in BFU-E Cells

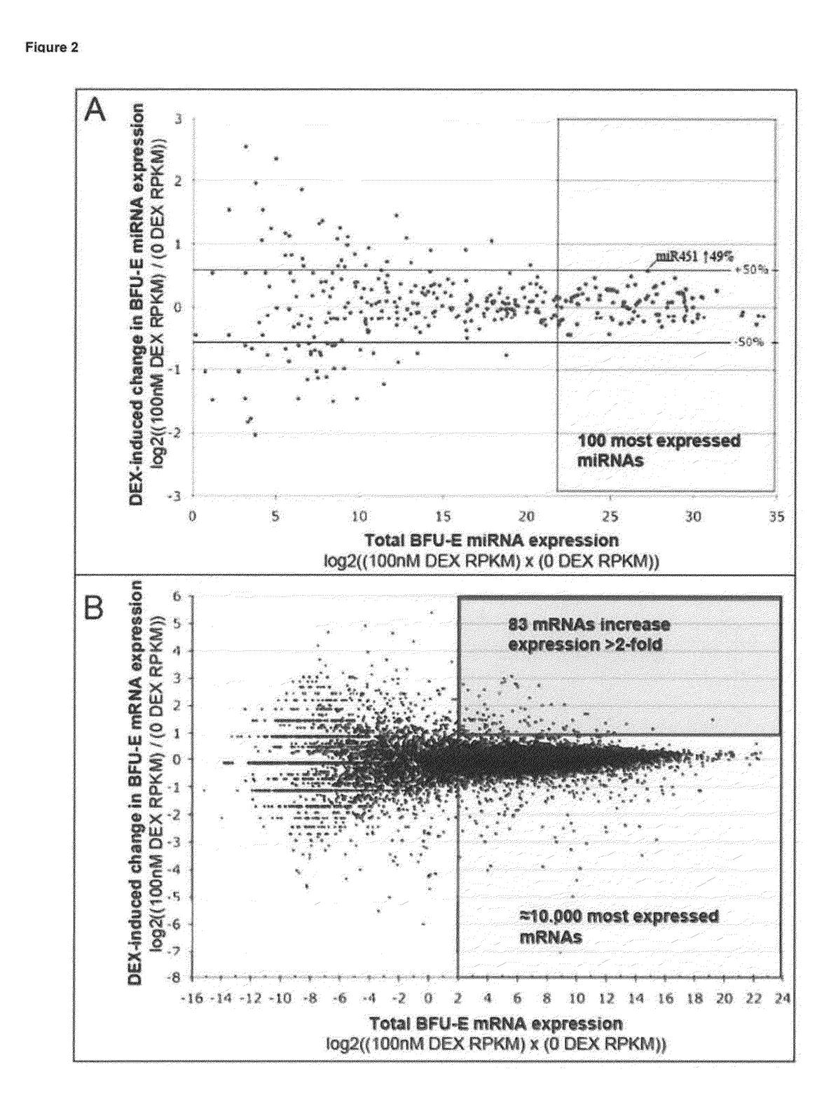

To determine at the molecular level how GCs stimulate BFU-E self-renewal, we employed next generation sequencing to determine the difference in relative expression of individual micro RNAs (miRNAs) and messenger RNAs (mRNAs) in BFU-E cells cultured 4 hours with or without 100 nM Dex. Differences in miRNA expression were determined as described previously (Baek et al., 2008). In cells treated with Dex none of the 100 highest expressed miRNAs increased expression more than 50%. miR-451 was changed the most with a 49% increase in expression (FIG. 2A). Since several miRNAs contain the same seed sequence and therefore target the same group of mRNAs we also compared changes in expression of miRNA families. None of the 50 miRNA families with the highest total expression values changed more than 50% upon Dex treatment (data not shown). The most abundant miRNA and miRNA families in BFU-E cells with and without Dex are represented in (FIGS. S4 B and C). Since only a large change in amount of a highly expressed miRNA family is likely to significantly change protein expression (Liang and Li, 2009), we conclude that GCs likely do not affect BFU-E cells primarily through changes in miRNA expression.

We next determined effects of Dex on mRNA expression. To minimize the risk of false positive GC target genes we chose to compare only highly expressed genes (≈10,000 genes with a multiplied RPKM count >4) (Mortazavi et al., 2008). We define GC target genes as those whose mRNA expression levels differ by more than a factor of 2 between BFU-E cells cultured 4 hours with and without Dex. By these stringent criteria only 195 genes are differentially expressed, of which 83 are up regulated and 112 are down regulated (FIG. 2B).

### Example 5

### BFU-E Cells Respond to Glucocorticoids by Increased Expression of Genes with Promoter Regions Containing Evolutionarily Conserved MYC and HIF1a Binding Sequences Rather than Glucocorticoid Response Elements

Most functionally relevant GCR target genes in BFU-E cells likely respond to Dex by increased rather than decreased expression (Reichardt et al., 1998; Wessely et al., 1997). We therefore focused on mechanisms of GC-induced transactivation in BFU-E cells, hoping that knowledge of this process would reveal strategies to pharmacologically stimulate this process.

In order to understand the cell specific context by which GCs activate transcription in BFU-E cells, we first evaluated computationally the promoter regions in the 83 upregulated genes. To this end, we first used Whole Genome rVISTA to determine if any transcription factor binding sites conserved from mouse to human were over-represented in genomic regions 5000 base pairs upstream of the 83 upregulated genes (FIG. S5). Surprisingly, there was no over-representation of glucocorticoid response elements (GREs) but instead a highly significant enrichment of predicted MYC (p-value=1×10⁻²⁴) and HIF1a binding sites (p-value=1×10⁻²⁷). This motif enrichment analysis was repeated with the 395 genes that were upregulated only 50% or more by Dex and with a multiplied RPKM count greater than zero. Again, in this larger set of less significantly enriched GC-target genes, the promoter segments were enriched for HIF1 (p-value=1×10⁻¹²) and MYC (p-value=1×10⁻⁹), but not for GRE sites (data not shown).

Motif enrichment analysis of −2000 to +200 bp promoter segments using Bayesian motif discovery approach implemented in webMOTIFS (Romer et al., 2007) confirms these results. Again no significant enrichment of GRE motifs was found, while the most significantly enriched motif (caCGTGgc) is both a possible MYC and HIF1a binding site (FIG. S6). In addition, we discovered that several of the upregulated genes that were not computationally predicted to be HIF1a targets have been experimentally validated as HIF1a targets in specific cell types (Table S3). The enrichment of MYC and HIF1a motifs in the promoter regions of upregulated genes prompted us to test whether a compound that enhances HIF1a activation enhances transcriptional activation of genes upregulated during stress erythropoiesis, and thereby stimulates CFU-E regeneration.

Although we were mostly interested in studying upregulated genes, it is interesting to note that gene ontology analysis of the ˜120 genes downregulated by Dex as well as by DMOG (data not shown) reveal a highly significant (p-values 1×10⁻⁹ to ×10⁻²⁰) downregulation of ribosomal protein genes and genes involved in nucleolar processes. Since ribosome biogenesis is regulated by mammalian target of rapamycin (mTOR), the shut down of ribosomal biogenesis could be caused by upregulation of an mTOR inhibitor. Indeed the gene most upregulated by Dex (>8-fold in 4 hours) is Ddit4 (Table S3), which counteracts the stimulatory effect of c-myc on mTOR activity and leads to reduced ribosome biogenesis (Gordan et al., 2007; Katiyar et al., 2009).

The transcriptional effects of GCR activation tend to be rather cell-specific (Rogatsky et al., 1997). The GCR belongs to a large family of ligand-inducible nuclear receptors that consist of ligand-binding, a DNA-binding, and three transactivation domains (Hittelman et al., 1999). There is one GCR gene in mice (Nr3C1) that is transcribed into a single mRNA. However, the GR mRNA transcript may produce several N-terminal isoforms with possible different functional characteristics, due to leaky ribosomal scanning or shunting and resulting in translation initiation at more distal in frame AUG codons (Lu and Cidlowski, 2005). Since the cell specific effect in BFU-E cells could depend on expression of a specific isoform in BFU-E and not CFU-E cells (Lu and Cidlowski, 2006), we performed Western blots but detected similar ratios of expression of at least four GCR isoforms in BFU-E and CFU-E cells, while Ter119+ erythroblasts express very low levels of GCR (data not shown). Therefore, without wishing to be bound by any theory, we suggest that the cell-specific action of GCR activation in BFU-E cells is more likely explained by BFU-E-specific availability of cofactors, other interacting transcription factors and epigenetic modifications (Fonte et al., 2007; Grad and Picard, 2007; John et al., 2008; Trousson et al., 2007).

Previous studies have suggested that in order to stimulate erythropoiesis the GCR must be able to dimerize (Bauer et al., 1999; Reichardt et al., 1998) and have a functional AF2 transactivation domain (Wessely et al., 1997) so as to activate transcription at GRE sites. Taking this data into account, we suggest that BFU-E self-renewal is induced by GCR transactivation rather than repression. In our study we found that only 83 genes were upregulated by Dex in BFU-E cells. Some have known important functions in erythroid progenitor proliferation such as the anti-apoptotic protein Bcl2 (Lacronique et al., 1997; Miyake et al., 2008), the protein tyrosine kinase Kit (Chabot et al., 1988) and the AML associated factor Trib2 (Keeshan et al., 2006).

To understand the mechanism of GCR-induced expression in BFU-E cells we evaluated computationally the promoter regions for enrichment of transcription factor motifs (FIGS. S5 and S6). The GCR is known to regulate transcription by chromatin interaction relatively far from the target genes (So et al., 2007). Remarkably, in A549 lung epithelial carcinoma cells 52% of Dex-induced genes and 92% of Dex-repressed genes show no GR binding within 10 kb of the transcription start site (Reddy et al., 2009). Even so, it was surprising that 83 significantly upregulated genes in BFU-E cells were not enriched for GRE sites in their promoter regions, but instead for MYC and HIF1 binding sites.

A cooperative effect between MYC and GCR in BFU-E cells would contrast with that in lymphoma and lymphoid leukemia cells, where GCs induce apoptosis by counteracting MYC activity (Frankfurt and Rosen, 2004). To confirm that the significant enrichment of MYC/HIF1a sites is not merely explained by the fact that highly expressed genes in BFU-E cells in general are enriched for MYC/HIF1a binding sites the enrichment analysis was repeated with 2847 well expressed (multiplied RPKM count >4) genes that change less than 7.2% (log 2(0.1)). Neither MYC, HIF1a or any other motifs were significantly enriched in these promoter regions (data not shown). The fact that GCs induce expression of genes in BFU-E cells that contain HIF1a motifs strongly suggests that these pathways cooperate in BFU-E self-renewal. Here we focused on the relationship between HIF1a and BFU-E self-renewal, rather than MYC, since HIF1a can be targeted by clinically tested PHIs and because, while not wishing to be bound by theory, we believe that it makes physiological sense that hypoxia would intrinsically enhance CFU-E production from BFU-E cells. We hypothesize a physiological scenario where GCs stimulate BFU-E self-renewal by increasing expression of genes that are enriched for proximal HIF1a binding sites and more distal GREs (not detected by our analysis) such that simultaneous exposure to systemic GCs and hypoxia enhances the expression in a synergistic manner.

It is also possible that GCs induce expression of HIF1a motif-enriched genes by direct GCR-HIF1 crosstalk leading to HIF1a stabilization and that these genes in fact are upregulated by GCR-dependent HIF1 activation. There is indeed evidence from non-erythroid cells fitting both theories. In non-erythroid cell lines activated GCR co-localizes with HIF1a and enhances HIF1a activation without altering HIF1a protein levels (Kodama et al., 2003), and hypoxia conversely enhances GRE promoter activity in reporter assays (Leonard et al., 2005). The mechanism of the observed GCR/HIF1a co-operation or crosstalk is unknown but may take place at the site of chromatin interaction where GCR and other transcription factors modulate each other's activity either in GRE-binding-dependent or GRE independent manners (Kassel and Herrlich, 2007).

### Example 6

### The Prolyl Hydroxylase Inhibitor Dimethyloxalylglycine (DMOG) Synergizes with Dex to Increase BFU-E Self-Renewal and CFU-E Production Through HIF1a Activation

We hypothesized that GC-dependent CFU-E regeneration could be enhanced by intrinsic HIF1a activation in BFU-E cells caused by prolyl-hydroxylase inhibitors (PHIs). PHIs act by inhibiting the oxygen-dependent degradation of HIF1a that is mediated by prolyl hydroxylases and are presently in clinical trials for inducing Epo production from kidneys in human subjects (Bunn, 2007). Since none of the recently developed HIF-specific PHIs are commercially available, we used the prototype PHI, dimethyloxalylglycine (DMOG) to test our hypothesis (Elvidge et al., 2006; Jaakkola et al., 2001).

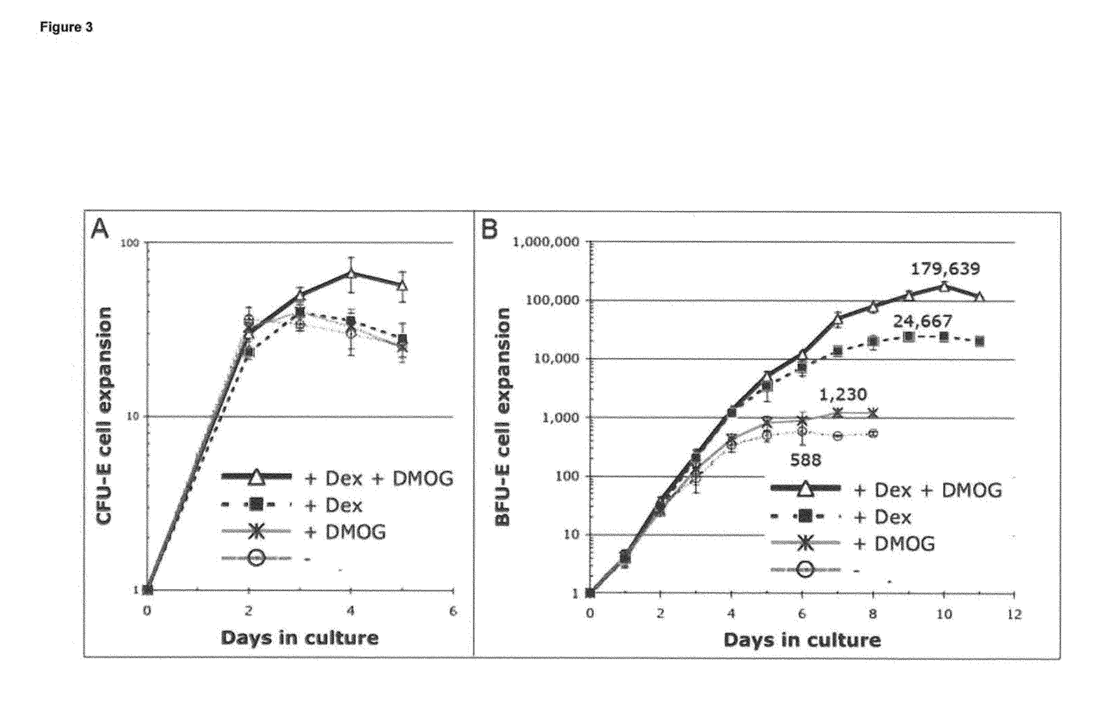

FIG. 3 indeed shows that DMOG synergizes with Dex to increase the erythroid output of BFU-E cells in liquid culture. DMOG alone stimulates proliferation of BFU-E cells 2-fold and, similar to the experiment in FIG. 1, Dex stimulates BFU-E proliferation 42-fold. Importantly, proliferation of BFU-E cells is stimulated 306-fold by a combination of Dex and DMOG. The synergistic effect of Dex and DMOG is shown by the fact that DMOG increases the stimulatory effect of Dex on BFU-E proliferation 7.3-fold. Recapitulating the results in FIG. 1, CFU-E cell proliferation is minimally affected by Dex. CFU-E cells do not increase proliferation in response to DMOG alone, and addition of both Dex and DMOG increases proliferation less than 2-fold (n=4).

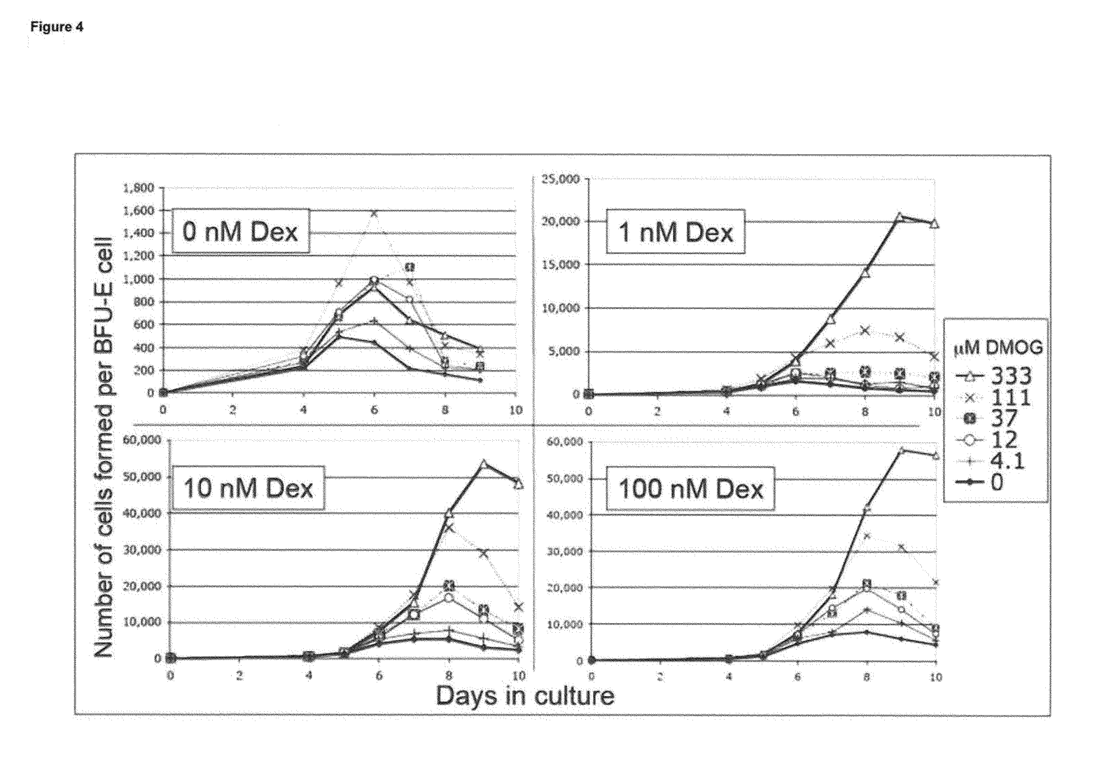

FIG. 4 shows that DMOG stimulates BFU-E expansion even in the presence of very low concentrations of Dex. Addition of 333 μM DMOG enhanced BFU-E proliferation 1.9-fold without Dex, 13-fold with 1 nM Dex, 10-fold with 10 nM Dex, and 7.3 fold with 100 nM Dex. These DMOG/Dex dose-response experiments clearly demonstrate that DMOG indeed is a very potent enhancer of CFU-E production from BFU-E cells, especially in the presence of low concentrations of Dex.

### Example 7

### DMOG Synergizes with Dexamethasone to Prevent BFU-E Cell Exhaustion, Allowing More CFU-E Cells to be Formed

In order to determine the intrinsic effects of DMOG and Dex, sorted CD24 and CD71^{10% low} BFU-E cells were cultured in SFELE medium with or without 100 nM Dex and with or without 333 μM DMOG. At daily intervals colony assays were performed (Table 2). As in the experiment in FIG. 3, for the first three days the total number of cells increased at the same rate in all cultures. Thereafter the numbers remained constant for cultures with no additions or with DMOG alone, whereas the numbers increased ˜20 fold in cultures with Dex alone and ˜120 fold in those containing both Dex and DMOG.

As in the experiment in Table 1, Dex alone increased generation of CFU-E cells at Day 3 of culture (51 CFU-Es per plated BFU-E versus 11) and the increase continued with time in culture. In contrast, in the cultures without Dex the numbers of CFU-Es remained constant. Similarly, Dex alone increased generation of BFU-E cells at Day 3 of culture (8.7 BFU-Es per plated BFU-E versus 2.2 in control cultures) and the number of BFU-E's remained constant until Day 5 of culture. In contrast in the cultures without Dex the numbers of BFU-Es then dropped precipitously.

Addition of DMOG alone somewhat increased the production of CFU-E cells (at Day 3, 24 CFU-Es were formed per plated BFU-E versus 11 in the control culture) and the number of CFU-E's remained constant for the next two days whereas it dropped in cultures without DMOG. Consistent with the notion that DMOG alone can somewhat stimulate self-renewal of BFU-E cells, at Day 3 of culture there were twice as many BFU-Es present (4.8 BFU-Es per plated BFU-E versus 2.2 in control cultures) and the number of BFU-Es did not decline as precipitously as in the control cultures (Table 2).

The most dramatic stimulatory effect on BFU-E self-renewal was seen in cultures containing both DMOG and Dex. At Day 3 of culture there were 2.4 (=21,146/8,734) times as many total BFU-Es than in cultures containing Dex alone, and the number of BFU-E's increased slightly by Day 5 (Table 2). In cultures containing both Dex and DMOG there was even self-renewal of the earliest Large BFU-Es—at Day 3 there were 1.6 Large BFU-Es per cell plated relative to 0.42 in the uncultured cells (Table 2).

Importantly, and compared to cultures containing Dex alone, in cultures containing both Dex and DMOG the number of CFU-E's were 1.9 fold higher at Day 4 (=269,975/138,600), 6.1 fold higher at Day 6 (=1,523,500/249,150), and 15 fold higher at Day 8 (=1,567,500/66,000). Thus DMOG synergizes with Dex in promoting BFU-E self-renewal, allowing over time more CFU-E cells and thus more differentiated erythroblasts to be formed than in cultures containing only Dex or only DMOG. The relative increase in CFU-E (and total erythroblast) output per BFU-E is 2.1 (2.5)-fold with DMOG alone, 26 (43)-fold with 100 nM Dex and 173 (313)-fold with DMOG and 100 nM Dex (Table 2), indicating a synergistic mode of action between HIF1a and GCR. In summary, Dex prevents exhaustion of early BFU-E cells and addition of DMOG enhances this effect.

### Example 8

### Proliferation of Erythroid Progenitors in the Lin-Sca-1-c-Kit+ Bone Marrow Progenitor Population is Synergistically Enhanced by DMOG and Dex

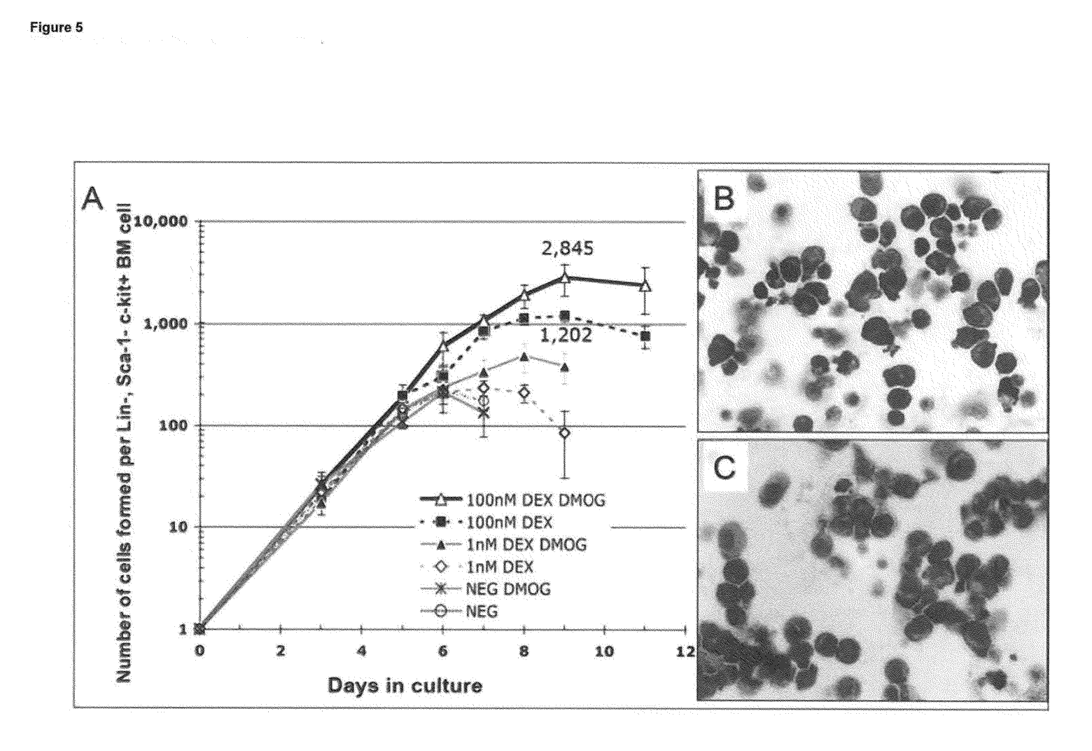

While we have not attempted to purify BFU-E cells from mouse bone marrow, the experiment in FIG. 5 shows that DMOG and Dex synergize to promote generation of erythroblasts from early bone marrow erythroid progenitors, presumably from BFU-E cells. In the bone marrow BFU-E cells are found in the Lin^{neg}, c-kit^{pos}, Sca-1^{neg} fraction; this cell population contains mostly a mix of lineage restricted progenitor cells of which a minority are BFU-Es (Pronk et al., 2007). We cultured these cells in SFELE medium with and without 1 nM or 100 nM Dex together or not with 333 μM DMOG. DMOG alone had little if any effect on proliferation of these cells, but significant increases were seen with 1 nM and 100 nM Dex. Importantly, DMOG enhances proliferation 2.1-fold and 2.4-fold above the effect of 1 nM and 10 nM Dex respectively. As FIG. 5b shows, bone marrow cells produced after 11 days of culture with 100 nM Dex and 333 μM DMOG had an erythroid morphology similar to that observed in an 11-day culture of fetal liver BFU-E cells in medium with 100 nM Dex and 333 μM DMOG.

### Example 9

### Maximum Erythroblast Formation from of Human Peripheral Blood Erythroid Progenitors is Synergistically Enhanced by DMOG and Dex

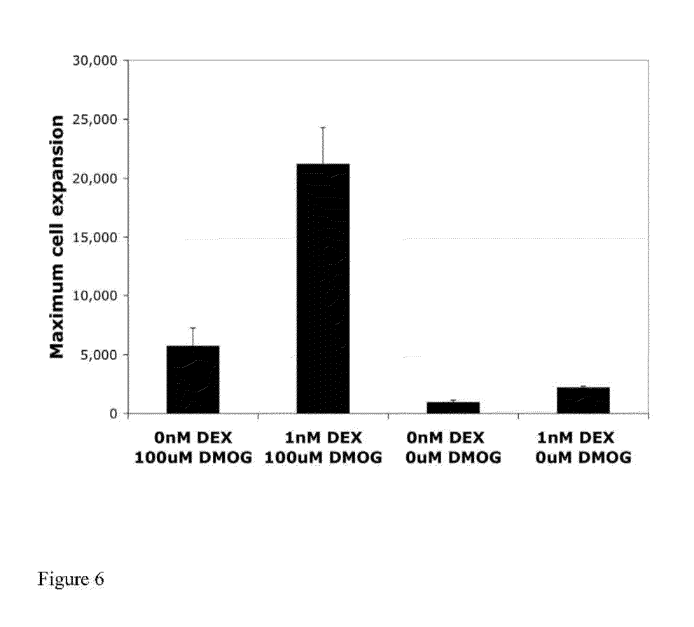

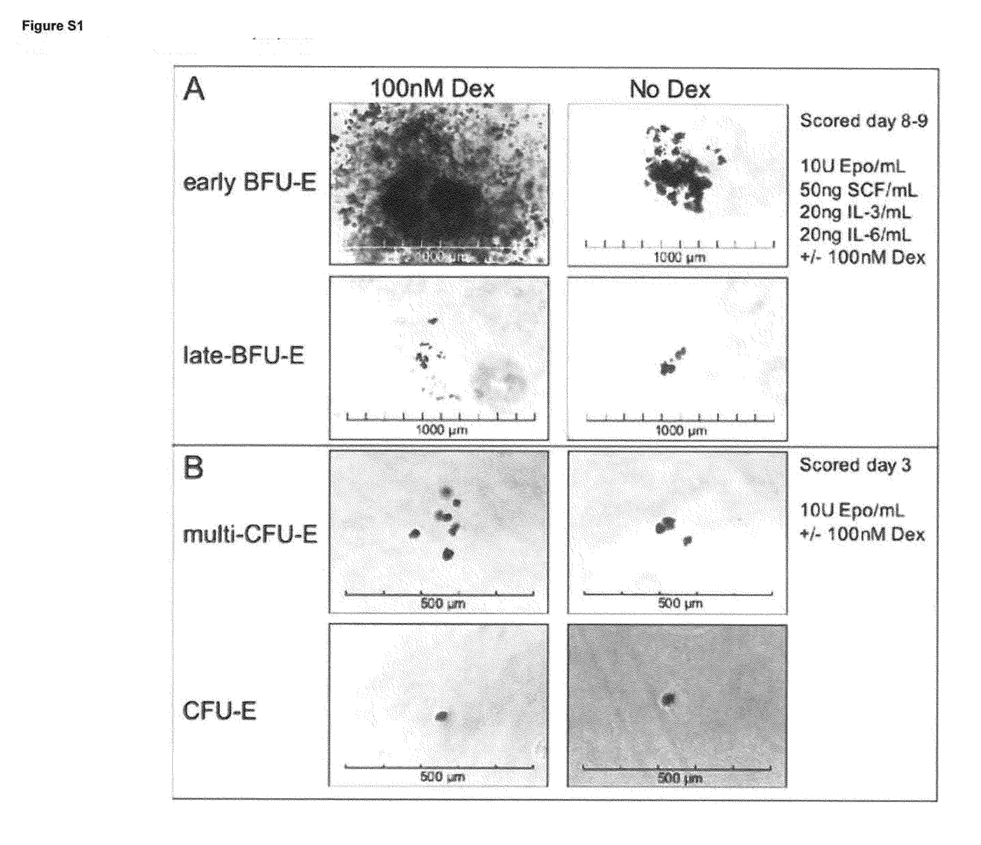

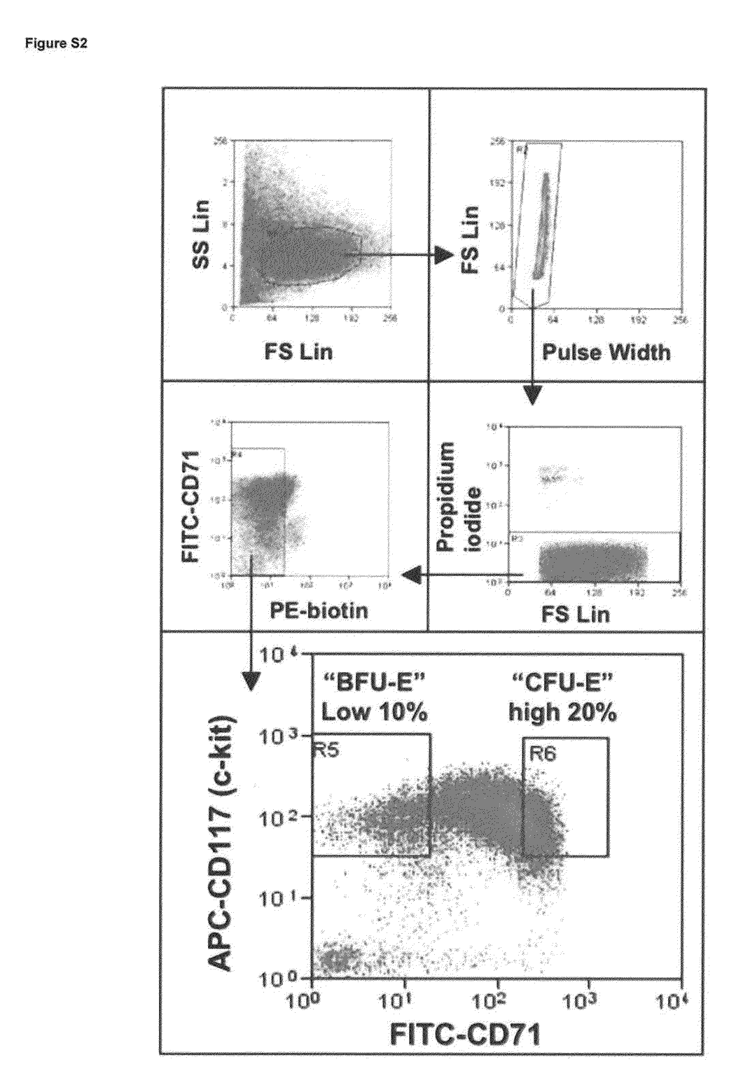

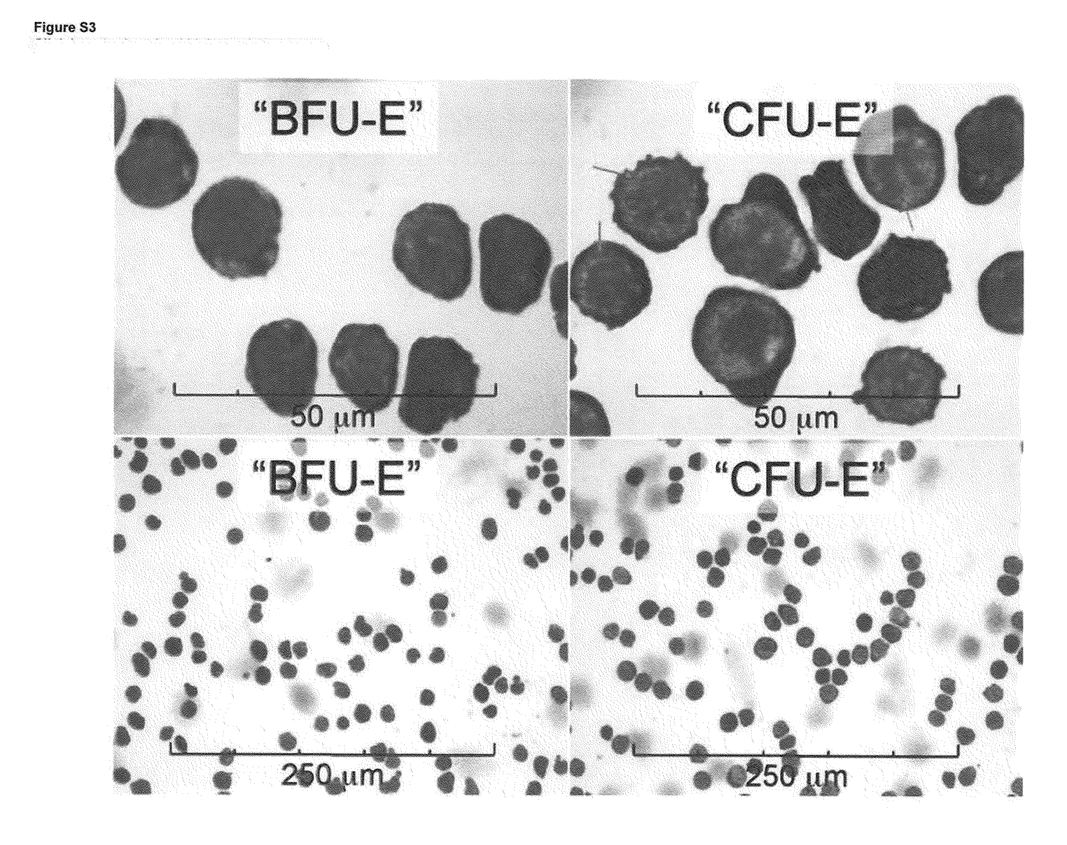

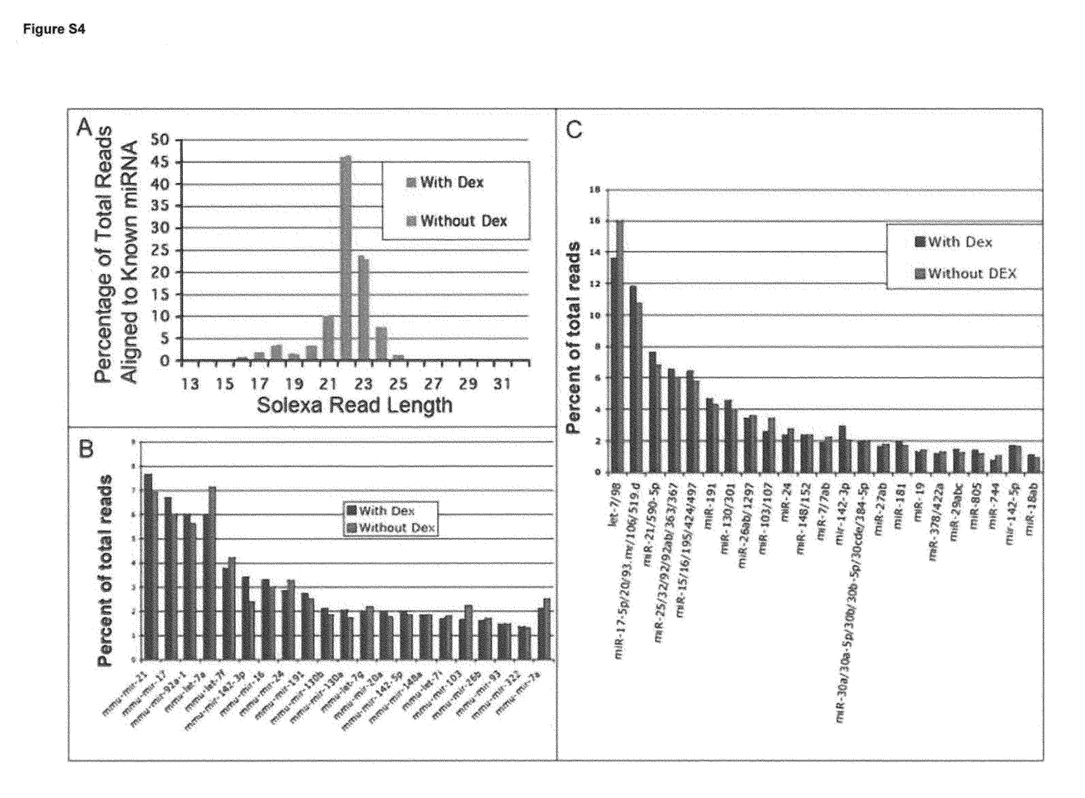

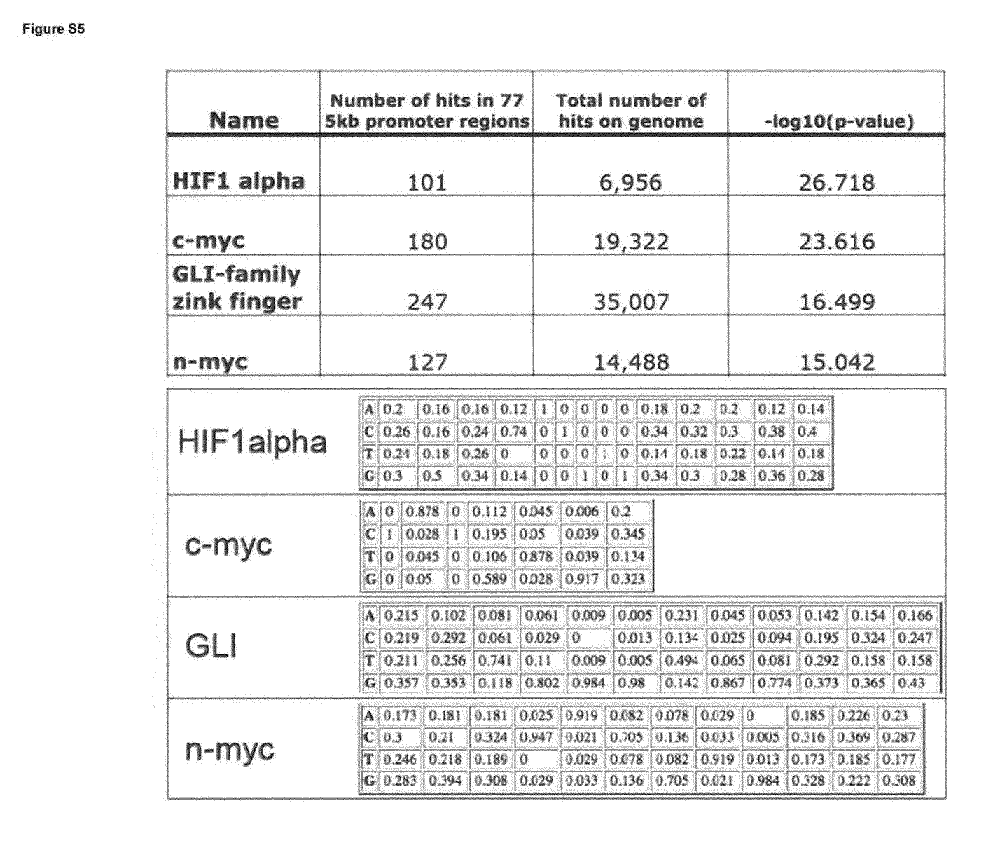

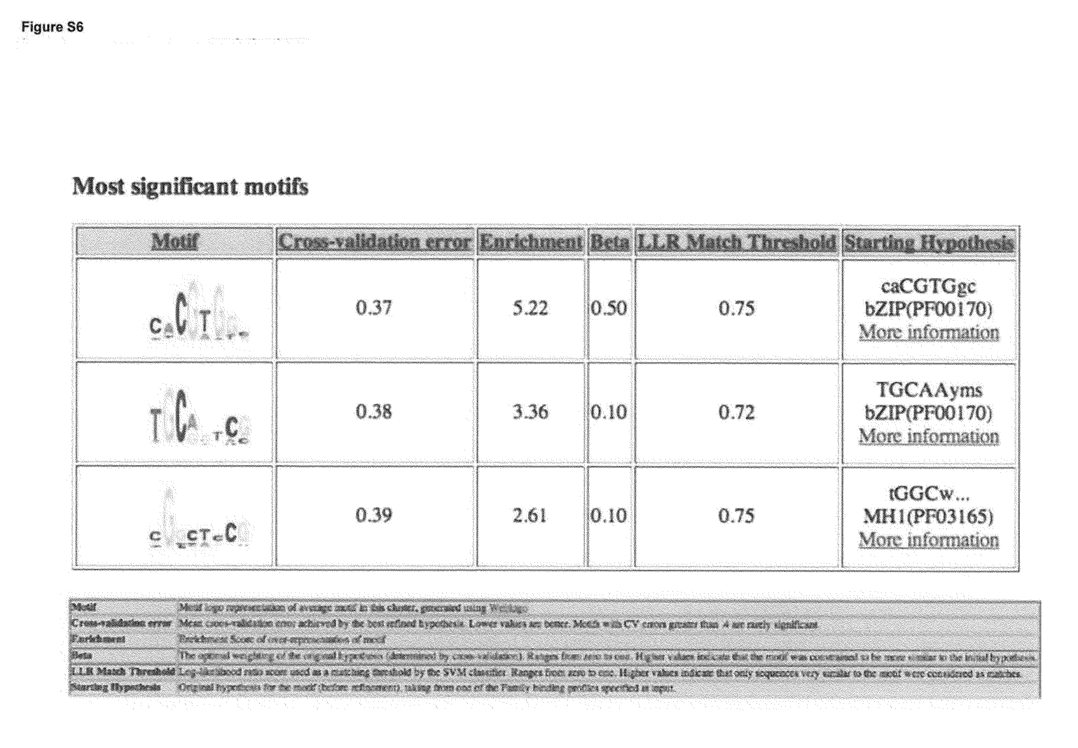

An experiment was performed on human hematopoietic progenitor cells from mobilized peripheral blood CD34+ cells that were first cultured 5 days in serum-free medium containing rhSCF, rhFlt-3L, rhIL-3 and rhIL-6. This mix of human progenitor cells was subsequently cultured in a version of SFELE medium version containing human factors rhEpo (2 U), rhSCF (100 ng/ml), and rhIGF-1 (40 ng/ml) with and without 1 nM Dex together or not with 100 μM DMOG. Similarly to the murine cultures >95% of cells at the end of the culture are erythroblasts (data not shown). Thus DMOG enhances the effect of Dex in human erythroid progenitors, allowing formation of ˜10-times more erythroblasts than in cultures with Dex alone (FIG. 6).

Discussion

As noted above, HIF-1 is composed of two subunits ARNT (HIFb) and HIF1a, HIF2a or HIF3a. ARNT assists in DNA binding, while transactivation is mediated through the two transactivation domains on HIF1a or HIF2, while HIF3a lacks a c-terminal transactivation domain and thereby inhibits HIF-mediated activation (Lando et al., 2002). Transcriptional activation by HIF1a is partly regulated by the oxygen-dependent HIF prolyl hydroxylases (Egln1, 2 and 3) (Jaakkola et al., 2001). These enzymes sense intracellular oxygen tension and use dioxygen as a substrate to hydroxylate a proline residue on HIF1a, which leads to its polyubiqutination by von Hippel Lindau protein and degradation by the 26S proteasome. HIF1a can also be inactivated by factor inhibiting HIF (FIH), which hydroxylates an asparagine residue in the C-terminal transactivation domain and thereby blocks the interaction of HIF with the transcriptional coactivator p300. Interestingly, HIF1-activity can be enhanced by sequestration of FIH by Inhibitor of NF-kBa (NfkbIa) (Shin et al., 2009). NfkbIa expression is induced by GCR binding at two GRE sites in the first intron (thus not detected in our enrichment analysis) (Auphan et al., 1995; Reddy et al., 2009), and is one of the 83 genes upegulated by Dex in BFU-E cells (Table S3). One possible explanation of the finding that Dex increases expression of HIF1a regulated genes in BFU-E cells is that Dex acts through Nfkbia to decrease asparaganine hydroxylation of HIF1a and thus increases HIF1a activation. Simultaneous inactivation of prolyl hydroxylation by DMOG would then inhibit both major mechanisms of HIF1a inactivation.

In BFU-E cells, GCs also induce expression of two negative HIF1 regulators; Egln3 (HIF-prolyl hydroxylase 3) and Hif3a (Table S3), likely because these are part of a negative feedback regulatory circuit in HIF-1-mediated signaling, and contain functional HIF1a-binding sites (Makino et al., 2007; Pescador et al., 2005).

We show that while a combination of Dex and DMOG had very little intrinsic effect on proliferation of CFU-E cells (FIG. 2A), the effect on BFU-E cells resulted in an amazing 173-fold increase in Epo-responsive CFU-E progenitors by day 7 (Table 2). Thus the experiments in Table 2 indicate that during a given BFU-E cell division, the presence of DMOG and Dex increases the probability that a BFU-E will divide and form daughter BFU-E cells (“self-renew”), rather than form more mature CFU-E cells (“differentiate”). To show that our findings are not restricted to erythroid progenitors from fetal liver, we repeated the in vitro proliferation cultures using Lin-, Sca-1-, c-kit+ BM cells. Although this population likely contains relatively much fewer BFU-E cells than our enriched fetal liver BFU-E cell populations, (Pronk et al., 2007) DMOG enhances also bone marrow-derived erythroblast formation in a GC-dependent manner (FIG. 5).

Importantly our dose-response curves using Dex, which has a longer half-life and is a ˜30-fold more potent GCR agonist than cortisol (West et al., 1960), are readily converted to physiological cortisol levels. While day-time free cortisol levels are normally 14-15 nM (corresponding to 0.43-0.45 nM Dex), levels increase to 98 nM (3.0 nM Dex) after surgery (Vogeser et al., 2003), and reach up to 120 nM (3.6 nM Dex) during septic shock (Torpy and Ho, 2007). Our dose response curves (in the absence of DMOG) show that normal GC concentrations (0.45 nM Dex) do not increase CFU-E formation, while a clear effect is seen at GC-levels detected during stress (>2 nM Dex), with a maximum effect obtained at concentrations above 10 nM Dex, which is equivalent to the concentrations in a patient treated with Prednisone (FIGS. 1A and B).

The therapeutic potential of activating intrinsic HIF1a in BFU-Es has to our knowledge never been discussed previously. As assessed by deep mRNA sequencing, HIF1a is highly expressed in purified BFU-E cells while expression of HIF2a is close to undetectable (data not shown). This agrees with previous findings that HIF2a is mainly expressed in endothelial cells (Tian et al., 1997). Without wishing to be bound by theory, this suggests that HIF1a may play a more important role than HIF2a in intrinsically enhancing BFU-E self-renewal. Although DMOG inhibits HIF prolyl and asparaginyl hydroxylases as well as collagen prolyl 4-hydroxylases the stimulatory effects on CFU-E regeneration are most likely not related to increased collagen prolyl hydroxylation, in part because collagen prolyl 4-hydroxylase alpha subunits are expressed at very low levels (Table S2). We therefore expect that the clinically tested HIF-specific PHIs that are active in the nanomolar range should also have a stimulatory effect on BFU-E proliferation similar to or better than that of DMOG.

A group of patients that would likely benefit from the synergistic CFU-E-promoting effect of PHIs and GCs are those with the red cell progenitor disorder Diamond-Blackfan Anemia (DBA) (Flygare and Karlsson, 2007; Vlachos et al., 2008). DBA patients have a severe deficiency of Epo-responsive CFU-E cells, while the few remaining CFU-Es undergo normal terminal erythroid differentiation. The fact that the only effective pharmacological treatment for these patients is a GC (Prednisone) suggests that DBA patients are very good candidates for evaluating the clinical benefits of PHI-induced generation of CFU-Es. This is supported by preliminary findings showing a synergistic effect of DMOG and Dex on BFU-E formation in CD34+ DBA patient cells in vitro. While our results may suggest that a therapeutic effect of PHIs would be dramatically enhanced by simultaneous administration of GCs, we note that, in the presence of only 1 nM Dex, addition of DMOG results in a 12-fold increase in erythroblast production (FIG. 4). Thus in vivo PHIs could enhance CFU-E-regeneration in synergy with endogenous cortisol levels or dramatically reduce the dose of prednisone needed for a therapeutic effect.

Without wishing to be bound by theory, we propose a revised physiological model of SE. In contrast to steady-state erythropoiesis, regulation of SE does not involve Epo-dependent CFU-E progenitors and erythroblasts. Instead, the increased numbers of erythroblasts observed during SE are the product of increased BFU-E self-renewal, caused by the combination of an increase in the systemic level of the stress hormone cortisol together with local anoxia. This leads to activation of the stress transcription factors GCR and HIF1a, which then synergize to maintain BFU-E progenitor immaturity during cell division, allowing over time increased numbers of divisions of individual BFU-E cells and thus increased numbers of CFU-E cells to be formed from each BFU-E. By showing that DMOG dramatically enhances this process we introduce the new concept of therapeutically enhancing red blood cell production already from the BFU-E stage.

Experimental Procedures (Please Note that Some Experimental Procedures are Described in Figure Legends and/or in the Examples Text Above)

Enrichment of Fetal Liver Erythroid Progenitors by Magnetic Depletion

E13.5-14.5 fetal liver cells were incubated with a cocktail of biotin-labeled lineage antibodies (mouse lineage panel, anti-mouse Ter119; CD16+CD32; Sca-1 and CD41) After magnetic depletion of positive cells a pure fetal liver erythroid progenitor (here called FLEP) population was obtained. Further details are described in Example 1.

Flow Cytometry Cell Sorting

BFU-E and CFU-E cells were separated from FLEP cells by flow cytometry. The “CFU-E” fraction is the 20% highest CD71 (and/or CD24a) expressing part of the c-kit+ fraction and the “BFU-E” fraction is the 10% lowest CD71 (and/or CD24a) expressing part. Around 100,000 “BFU-E” and 200,000 “CFU-E” cells were isolated per pregnant female. Further details are described in Example 1.

Serum Free Erythroid Liquid Expansion (SFELE) Progenitor Growth Assay

We developed a modified version of the serum free erythroid liquid expansion culture (SFELE) medium described by Dolzning et al. (Dolznig et al., 2006). Our complete erythroid expansion medium is 100 ng/mL rmSCF, 40 ng/mL rmIGF-1 and 2U/mL rhEPO, in StemSpan® SFEM, with or without Dex and/or DMOG.

Colony-Forming Assays

For CFU-E colony forming assays 1000 cells were plated in MethoCult M3234 (StemCell Technologies) containing 10U rhEPO, with or without 100 nM Dex. Colonies were scored after 3 days. The BFU-E assays were performed in MethoCult M3234 containing 10U rhEPO, 20 ng/mL rmIL-3, 20 ng/mL rmIL-6 and 50 ng/mL rmSCF, (PeproTech, Inc), with or without 100 nM Dex. The number of hemoglobinized early BFU-E and late BFU-E colonies was determined after 8-9 days following staining with 2,7-Diaminofluorene (Sigma Chemical, St. Louis, Mo.).

Cytospin Preparations and Histological Staining

First, 50,000 sorted in vitro cultured cells were centrifuged onto poly-lysine coated slides for 3 minutes at 500 rpm (Cytospin 3; Thermo Shandon, Pittsburgh, Pa.). Cells were air dried and fixed in −20° C. methanol for 2 minutes and stained with May-Grünewald Giemsa according to the manufacturer's recommendations (Sigma Chemical).

Small RNA Sequencing and mRNA-Seq

Small RNA sequencing was performed essentially as described (Grimson et al., 2008). Total RNAs from samples of BFU-E cells cultured 4 hours with or without 100 nM Dex were extracted with miRNeasy mini kit (Qiagen). miRNA-Seq reads with the adaptor starting after 15 bases were identified. The adaptor sequence was stripped and aligned to mouse NCBI build 37 (mm9) using ELAND. Reads mapping to the known microRNA hairpins were used for downstream analysis. Reads mapping to miRNA with more than one known copy in the genome were divided equally between the different loci. miRNA analysis methods to compute miRNA expression counts were derived from previously published methods (Baek et al., 2008). The read length distribution for solexa reads mapping to known miRNA hairpins is the same for BFU-E cells with and without Dex (FIG. S4 A).

Samples for Paired-End mRNA-Seq were prepared using the Solexa kit according to the manufacturer's instructions with the exception that we extracted 300 bp bands, before and after the PCR step (Illumina). Images acquired from the Solexa sequencer were processed through the bundled Solexa image extraction pipeline version 1.4. mRNA-Seq reads were aligned to mouse NCBI build 37 (mm9) using ELAND, Briefly, the first 32 bases of a read were used as a seed. Each matched seed was then extended up to 36 bases and scored to break any ties between multi-matches. For mRNA expression counts, unique reads in the genome that landed within any exons of NCBI gene models (v37.1) were counted. The counts were normalized by the mRNA length to get the final RPKM values (i.e. reads per KB per million reads).

All sequence data has been uploaded to the NCBI GEO database.

Motif Enrichment Analysis

Whole Genome rVista tool was used to identify transcription factor binding sites that are conserved between species and enriched in upstream regions of genes unregulated in BFU-E with Dex vs without Dex. In addition, THEME algorithm implemented in the web tool webMOTIF was used to identify significant motif's present in the genes unregulated in BFU-E with Dex vs without Dex.

The paper by Flygare J, Rayon Estrada V, Shin C, Gupta S, Lodish H F., entitled “HIF-1alpha synergizes with glucocorticoids to promote BFU-E progenitor self-renewal”, Blood. Published online Dec. 21, 2010 (PMID: 21177435), and all references cited therein, is incorporated herein by reference.

### REFERENCES

Acosta, J. C., Ferrandiz, N., Bretones, G., Torrano, V., Blanco, R., Richard, C., O'Connell, B., Sedivy, J., Delgado, M. D., and Leon, J. (2008). Myc inhibits p27-induced erythroid differentiation of leukemia cells by repressing erythroid master genes without reversing p27-mediated cell cycle arrest. Mol Cell Biol 28, 7286-7295.
Ang, S. O., Chen, H., Hirota, K., Gordeuk, V. R., Jelinek, J., Guan, Y., Liu, E., Sergueeva, A. I., Miasnikova, G. Y., Mole, D., et al. (2002). Disruption of oxygen homeostasis underlies congenital Chuvash polycythemia. Nat Genet 32, 614-621.
Auphan, N., DiDonato, J. A., Rosette, C., Helmberg, A., and Karin, M. (1995). Immunosuppression by glucocorticoids: inhibition of NF-kappa B activity through induction of I kappa B synthesis. Science 270, 286-290.
Baek, D., Villen, J., Shin, C., Camargo, F. D., Gygi, S. P., and Bartel, D. P. (2008). The impact of microRNAs on protein output. Nature 455, 64-71.
Bauer, A., Tronche, F., Wessely, O., Kellendonk, C., Reichardt, H. M., Steinlein, P., Schutz, G., and Beug, H. (1999). The glucocorticoid receptor is required for stress erythropoiesis. Genes Dev 13, 2996-3002.
Bunn, H. F. (2007). New agents that stimulate erythropoiesis. Blood 109, 868-873. Chabot, B., Stephenson, D. A., Chapman, V. M., Besmer, P., and Bernstein, A. (1988). The proto-oncogene c-kit encoding a transmembrane tyrosine kinase receptor maps to the mouse W locus. Nature 335, 88-89.
Dolznig, H., Grebien, F., Deiner, E. M., Stangl, K., Kolbus, A., Habermann, B., Kerenyi, M. A., Kieslinger, M., Moriggl, R., Beug, H., et al. (2006). Erythroid progenitor renewal versus differentiation: genetic evidence for cell autonomous, essential functions of EpoR, Stat5 and the GR. Oncogene 25, 2890-2900.
Elvidge, G. P., Glenny, L., Appelhoff, R. J., Ratcliffe, P. J., Ragoussis, J., and Gleadle, J. M. (2006). Concordant regulation of gene expression by hypoxia and 2-oxoglutarate-dependent dioxygenase inhibition: the role of HIF-1alpha, HIF-2alpha, and other pathways. J Biol Chem 281, 15215-15226.
Flygare, J., and Karlsson, S. (2007). Diamond-Blackfan anemia: erythropoiesis lost in translation. Blood 109, 3152-3154.
Fonte, C., Trousson, A., Grenier, J., Schumacher, M., and Massaad, C. (2007). Opposite effects of CBP and p300 in glucocorticoid signaling in astrocytes. J Steroid Biochem Mol Biol 104, 220-227.
Frankfurt, O., and Rosen, S. T. (2004). Mechanisms of glucocorticoid-induced apoptosis in hematologic malignancies: updates. Curr Opin Oncol 16, 553-563.
Gordan, J. D., Thompson, C. B., and Simon, M. C. (2007). HIF and c-Myc: sibling rivals for control of cancer cell metabolism and proliferation. Cancer cell 12, 108-113.
Grad, I., and Picard, D. (2007). The glucocorticoid responses are shaped by molecular chaperones. Mol Cell Endocrinol 275, 2-12.
Grimson, A., Srivastava, M., Fahey, B., Woodcroft, B. J., Chiang, H. R., King, N., Degnan, B. M., Rokhsar, D. S., and Bartel, D. P. (2008). Early origins and evolution of microRNAs and Piwi-interacting RNAs in animals. Nature 455, 1193-1197.
Heath, D. S., Axelrad, A. A., McLeod, D. L., and Shreeve, M. M. (1976). Separation of the erythropoietin-responsive progenitors BFU-E and CFU-E in mouse bone marrow by unit gravity sedimentation. Blood 47, 777-792.
Hickey, M. M., Lam, J. C., Bezman, N. A., Rathmell, W. K., and Simon, M. C. (2007). von Hippel-Lindau mutation in mice recapitulates Chuvash polycythemia via hypoxia-inducible factor-2alpha signaling and splenic erythropoiesis. J Clin Invest 117, 3879-3889.
Hittelman, A. B., Burakov, D., Iniguez-Lluhi, J. A., Freedman, L. P., and Garabedian, M. J. (1999). Differential regulation of glucocorticoid receptor transcriptional activation via AF-1-associated proteins. EMBO J 18, 5380-5388.
Jaakkola, P., Mole, D. R., Tian, Y. M., Wilson, M. I., Gielbert, J., Gaskell, S. J., Kriegsheim, A., Hebestreit, H. F., Mukherji, M., Schofield, C. J., et al. (2001). Targeting of HIF-alpha to, the von Hippel-Lindau ubiquitylation complex by O2-regulated prolyl hydroxylation. Science 292, 468-472.
John, S., Sabo, P. J., Johnson, T. A., Sung, M. H., Biddie, S. C., Lightman, S. L., Voss, T. C., Davis, S. R., Meltzer, P. S., Stamatoyannopoulos, J. A., et al. (2008). Interaction of the glucocorticoid receptor with the chromatin landscape. Mol Cell 29, 611-624.
Kassel, O., and Herrlich, P. (2007). Crosstalk between the glucocorticoid receptor and other transcription factors: molecular aspects. Mol Cell Endocrinol 275, 13-29.
Katiyar, S., Liu, E., Knutzen, C. A., Lang, E. S., Lombardo, C. R., Sankar, S., Toth, J. I., Petroski, M. D., Ronai, Z., and Chiang, G. G. (2009). REDD1, an inhibitor of mTOR signalling, is regulated by the CUL4A-DDB1 ubiquitin ligase. EMBO reports 10, 866-872.
Keeshan, K., He, Y., Wouters, B. J., Shestova, O., Xu, L., Sai, H., Rodriguez, C. G., Maillard, I., Tobias, J. W., Valk, P., et al. (2006). Tribbles homolog 2 inactivates C/EBPalpha and causes acute myelogenous leukemia. Cancer cell 10, 401-411.
Kodama, T., Shimizu, N., Yoshikawa, N., Makino, Y., Ouchida, R., Okamoto, K., Hisada, T., Nakamura, H., Morimoto, C., and Tanaka, H. (2003). Role of the glucocorticoid receptor for regulation of hypoxia-dependent gene expression. J Biol Chem 278, 33384-33391.
Kolbus, A., Blazquez-Domingo, M., Carotta, S., Bakker, W., Luedemann, S., von Lindern, M., Steinlein, P., and Beug, H. (2003). Cooperative signaling between cytokine receptors and the glucocorticoid receptor in the expansion of erythroid progenitors: molecular analysis by expression profiling. Blood 102, 3136-3146.
Lacronique, V., Varlet, P., Mayeux, P., Porteu, A., Gisselbrecht, S., Kahn, A., and Lacombe, C. (1997). Bcl-2 targeted overexpression into the erythroid lineage of transgenic mice delays but does not prevent the apoptosis of erythropoietin-deprived erythroid progenitors. Blood 90, 3050-3056.
Lando, D., Peet, D. J., Whelan, D. A., Gorman, J. J., and Whitelaw, M. L. (2002). Asparagine hydroxylation of the HIF transactivation domain a hypoxic switch. Science 295, 858-861.
Leonard, M. O., Godson, C., Brady, H. R., and Taylor, C. T. (2005). Potentiation of glucocorticoid activity in hypoxia through induction of the glucocorticoid receptor. J Immunol 174, 2250-2257.
Liang, H., and Li, W. H. (2009). Lowly expressed human microRNA genes evolve rapidly. Mol Biol Evol 26, 1195-1198.
Lu, N. Z., and Cidlowski, J. A. (2005). Translational regulatory mechanisms generate N-terminal glucocorticoid receptor isoforms with unique transcriptional target genes. Mol Cell 18, 331-342.
Lu, N. Z., and Cidlowski, J. A. (2006). Glucocorticoid receptor isoforms generate transcription specificity. Trends Cell Biol 16, 301-307.
Makino, Y., Uenishi, R., Okamoto, K., Isoe, T., Hosono, O., Tanaka, H., Kanopka, A., Poellinger, L., Haneda, M., and Morimoto, C. (2007). Transcriptional up-regulation of inhibitory PAS domain protein gene expression by hypoxia-inducible factor 1 (HIF-1): a negative feedback regulatory circuit in HIF-1-mediated signaling in hypoxic cells. J Biol Chem 282, 14073-14082.
Miyake, K., Utsugisawa, T., Flygare, J., Kiefer, T., Hamaguchi, I., Richter, J., and Karlsson, S. (2008). Ribosomal protein S19 deficiency leads to reduced proliferation and increased apoptosis but does not affect terminal erythroid differentiation in a cell line model of Diamond-Blackfan anemia. Stem Cells 26, 323-329.
Mortazavi, A., Williams, B. A., McCue, K., Schaeffer, L., and Wold, B. (2008). Mapping and quantifying mammalian transcriptomes by RNA-Seq. Nat Methods 5, 621-628.
Nijhof, W., and Wierenga, P. K. (1983). Isolation and characterization of the erythroid progenitor cell: CFU-E. J Cell Biol 96, 386-392.
Perry, J. M., Harandi, O. F., and Paulson, R. F. (2007). BMP4, SCF, and hypoxia cooperatively regulate the expansion of murine stress erythroid progenitors. Blood 109, 4494-4502.
Pescador, N., Cuevas, Y., Naranjo, S., Alcaide, M., Villar, D., Landazuri, M. O., and Del Peso, L. (2005). Identification of a functional hypoxia-responsive element that regulates the expression of the egl nine homologue 3 (egln3/phd3) gene. Biochem J 390, 189-197.
Pronk, C. J., Rossi, D. J., Mansson, R., Attema, J. L., Norddahl, G. L., Chan, C. K., Sigvardsson, M., Weissman, I. L., and Bryder, D. (2007). Elucidation of the phenotypic, functional, and molecular topography of a myeloerythroid progenitor cell hierarchy. Cell Stem Cell 1, 428-442.
Reddy, T. E., Pauli, F., Sprouse, R. O., Neff, N. F., Newberry, K. M., Garabedian, M. J., and Myers, R. M. (2009). Genomic determination of the glucocorticoid response reveals unexpected mechanisms of gene regulation. Genome Res 19, 2163-2171.
Reichardt, H. M., Kaestner, K. H., Tuckermann, J., Kretz, O., Wessely, O., Bock, R., Gass, P., Schmid, W., Herrlich, P., Angel, P., et al. (1998). DNA binding of the glucocorticoid receptor is not essential for survival. Cell 93, 531-541.
Rogatsky, I., Trowbridge, J. M., and Garabedian, M. J. (1997). Glucocorticoid receptor-mediated cell cycle arrest is achieved through distinct cell-specific transcriptional regulatory mechanisms. Mol Cell Biol 17, 3181-3193.
Romer, K. A., Kayombya, G. R., and Fraenkel, E. (2007). WebMOTIFS: automated discovery, filtering and scoring of DNA sequence motifs using multiple programs and Bayesian approaches. Nucleic Acids Res 35, W217-220.
Sawada, K., Krantz, S. B., Dai, C. H., Koury, S. T., Horn, S. T., Glick, A. D., and Civin, C. I. (1990). Purification of human blood burst-forming units-erythroid and demonstration of the evolution of erythropoietin receptors. J Cell Physiol 142, 219-230.
Shin, D. H., Li, S. H., Yang, S. W., Lee, B. L., Lee, M. K., and Park, J. W. (2009). Inhibitor of nuclear factor-kappaB alpha derepresses hypoxia-inducible factor-1 during moderate hypoxia by sequestering factor inhibiting hypoxia-inducible factor from hypoxia-inducible factor 1alpha. FEBS J 276, 3470-3480.
Smith, M. J., Charron-Prochownik, D. C., and Prochownik, E. V. (1990). The leucine zipper of c-Myc is required for full inhibition of erythroleukemia differentiation. Mol Cell Biol 10, 5333-5339.
So, A. Y., Chaivorapol, C., Bolton, E. C., Li, H., and Yamamoto, K. R. (2007). Determinants of cell- and gene-specific transcriptional regulation by the glucocorticoid receptor. PLoS Genet 3, e94.
Terszowski, G., Waskow, C., Conradt, P., Lenze, D., Koenigsmann, J., Carstanjen, D., Horak, I., and Rodewald, H. R. (2005). Prospective isolation and global gene expression analysis of the erythrocyte colony-forming unit (CFU-E). Blood 105, 1937-1945.
Tian, H., McKnight, S. L., and Russell, D. W. (1997). Endothelial PAS domain protein 1 (EPAS1), a transcription factor selectively expressed in endothelial cells. Genes Dev 11, 72-82.
Torpy, D. J., and Ho, J. T. (2007). Value of free cortisol measurement in systemic infection. Horm Metab Res 39, 439-444.
Trousson, A., Grenier, J., Fonte, C., Massaad-Massade, L., Schumacher, M., and Massaad, C. (2007). Recruitment of the p160 coactivators by the glucocorticoid receptor: dependence on the promoter context and cell type but not hypoxic conditions. J Steroid Biochem Mol Biol 104, 305-311.
Vlachos, A., Ball, S., Dahl, N., Alter, B. P., Sheth, S., Ramenghi, U., Meerpohl, J., Karlsson, S., Liu, J. M., Leblanc, T., et al. (2008). Diagnosing and treating Diamond Blackfan anaemia: results of an international clinical consensus conference. Br J Haematol 142, 859-876.
Vogeser, M., Groetzner, J., Kupper, C., and Briegel, J. (2003). Free serum cortisol during the postoperative acute phase response determined by equilibrium dialysis liquid chromatography-tandem mass spectrometry. Clin Chem Lab Med 41, 146-151.
Wessely, O., Deiner, E. M., Beug, H., and von Lindern, M. (1997). The glucocorticoid receptor is a key regulator of the decision between self-renewal and differentiation in erythroid progenitors. EMBO J 16, 267-280.
West, K. M., Johnson, P. C., Kyriakopoulos, A. A., Bahr, W. J., and Bloedow, C. E. (1960). The physiologic effects of dexamethasone. Arthritis Rheum 3, 129-139.
Yoon, D., Pastore, Y. D., Divoky, V., Liu, E., Mlodnicka, A. E., Rainey, K., Ponka, P., Semenza, G. L., Schumacher, A., and Prchal, J. T. (2006). Hypoxia-inducible factor-1 deficiency results in dysregulated erythropoiesis signaling and iron homeostasis in mouse development. J Biol Chem 281, 25703-25711.
Heath, D. S., Axelrad, A. A., McLeod, D. L., and Shreeve, M. M. (1976). Separation of the erythropoietin-responsive progenitors BFU-E and CFU-E in mouse bone marrow by unit gravity sedimentation. Blood 47, 777-792.
Kaiho, S., and Mizuno, K. (1985). Sensitive assay systems for detection of hemoglobin with 2,7-diaminofluorene: histochemistry and colorimetry for erythrodifferentiation. Anal Biochem 149, 117-120.
McKearn, J. P., McCubrey, J., and Fagg, B. (1985). Enrichment of hematopoietic precursor cells and cloning of multipotential B-lymphocyte precursors. Proc Natl Acad Sci USA 82, 7414-7418.
Nielsen, P. J., Lorenz, B., Muller, A. M., Wenger, R. H., Brombacher, F., Simon, M., von der Weid, T., Langhorne, W. J., Mossmann, H., and Kohler, G. (1997). Altered erythrocytes and a leaky block in B-cell development in CD24/HSA-deficient mice. Blood 89, 1058-1067.
Nijhof, W., and Wierenga, P. K. (1983). Isolation and characterization of the erythroid progenitor cell: CFU-E. J Cell Biol 96, 386-392.
Pescador, N., Cuevas, Y., Naranjo, S., Alcaide, M., Villar, D., Landazuri, M. O., and Del Peso, L. (2005). Identification of a functional hypoxia-responsive element that regulates the expression of the egl nine homologue 3 (egln3/phd3) gene. Biochem J 390, 189-197.
Praloran, V., Klausman, M., Naud, M. F., and Harousseau, J. L. (1989). Blood erythroid progenitors (CFU-E and BFU-E) in acute lymphoblastic leukemias. Blut 58, 75-78.
Pronk, C. J., Rossi, D. J., Mansson, R., Attema, J. L., Norddahl, G. L., Chan, C. K., Sigvardsson, M., Weissman, I. L., and Bryder, D. (2007). Elucidation of the phenotypic, functional, and molecular topography of a myeloerythroid progenitor cell hierarchy. Cell Stem Cell 1, 428-442.
Sawada, K., Krantz, S. B., Dai, C. H., Koury, S. T., Horn, S. T., Glick, A. D., and Civin, C. I. (1990). Purification of human blood burst-forming units-erythroid and demonstration of the J evolution of erythropoietin receptors. J Cell Physiol 142, 219-230.
Terszowski, G., Waskow, C., Conradt, P., Lenze, D., Koenigsmann, J., Carstanjen, D., Horak, I., and Rodewald, H. R. (2005). Prospective isolation and global gene expression analysis of the erythrocyte colony-forming unit (CFU-E). Blood 105, 1937-1945.
Elebute, M. O., Rizzo, S., Tooze, J. A., Marsh, J. C., Gordon-Smith, E. C., and Gibson, F. M. (2003). Evaluation of the haemopoietic reservoir in de novo haemolytic paroxysmal nocturnal haemoglobinuria. Br J Haematol 123, 552-560.
Erslev, A. J. (2000). Erythropoietin and anemia of cancer. Eur J Haematol 64, 353-358.
Flygare, J., and Karlsson, S. (2007). Diamond-Blackfan anemia: erythropoiesis lost in translation. Blood 109, 3152-3154.
Gaman, A., Gaman, G., and Bold, A. (2009). Acquired aplastic anemia: correlation between etiology, pathophysiology, bone marrow histology and prognosis factors. Rom J Morphol Embryol 50, 669-674.
Morra, L., Moccia, F., Mazzarello, G. P., Bessone, G., Mela, G. S., and Ponassi, G. A. (1993). Defective in vitro growth of BFU-E of elderly subjects revealed by hydrocortisone. Biomed Pharmacother 47, 167-171.
Ru, Y. X., Mao, B. Y., Zhang, F. K., Pang, T. X., Zhao, S. X., Liu, J. H., and Wickramasinghe, S. N. (2009). Invasion of erythroblasts by Pasmodium vivax: A new mechanism contributing to malarial anemia. Ultrastruct Pathol 33, 236-242.
Spivak, J. L., Gascon, P., and Ludwig, H. (2009). Anemia management in oncology and hematology. Oncologist 14 Suppl 1, 43-56.

Those skilled in the art will recognize, or be able to ascertain using no more than routine experimentation, many equivalents to the specific embodiments of the invention described herein. The scope of the present invention is not intended to be limited to the embodiments described above. The invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Features disclosed herein with respect to any particular aspect or embodiment of the invention may be applied to other aspects and embodiments. The headings herein are for convenience and shall not limit the invention in any way.

Articles such as “a” and “an”, and the like, may mean one or more than one unless indicated to the contrary or otherwise evident from the context.

The phrase “and/or” as used herein in the specification and in the claims, should be understood to mean “either or both” of the elements so conjoined. Multiple elements listed with “and/or” should be construed in the same fashion, i.e., “one or more” of the elements so conjoined. Other elements may optionally be present other than the elements specifically identified by the “and/or” clause. As used herein in the specification and in the claims, “or” should be understood to have the same meaning as “and/or” as defined above. For example, when used in a list of elements, “or” or “and/or” shall be interpreted as being inclusive, i.e., the inclusion of at least one, but optionally more than one, of list of elements, and, optionally, additional unlisted elements. Only terms clearly indicative to the contrary, such as “only one of” or “exactly one of” will refer to the inclusion of exactly one element of a number or list of elements. Thus claims that include “or” between one or more members of a group are considered satisfied if one, more than one, or all of the group members are present, employed in, or otherwise relevant to a given product or process unless indicated to the contrary. The invention provides embodiments in which exactly one member of the group is present, employed in, or otherwise relevant to a given product or process. The invention also provides embodiments in which more than one, or all of the group members are present, employed in, or otherwise relevant to a given product or process. It is to be understood that the invention encompasses embodiments in which one or more limitations, elements, clauses, descriptive terms, etc., of a claim is introduced into another claim. For example, a claim that is dependent on another claim can be modified to include one or more elements or limitations found in any other claim that is dependent on the same base claim.

Where the claims recite a composition, it is understood that methods of using the composition as disclosed herein are provided, and methods of making the composition according to any of the methods of making disclosed herein are provided. Where the claims recite a method, it is understood that a composition for performing the method is provided. Where elements are presented as lists or groups, each subgroup is also disclosed. It should also be understood that, in general, where the invention, or aspects of the invention, is/are referred to as comprising particular elements, features, etc., certain embodiments of the invention or aspects of the invention consist of, or consist essentially of, such elements, features, etc.

It should also be understood that, unless clearly indicated to the contrary, in any methods claimed herein that include more than one step or act, the order of the steps or acts of the method is not necessarily limited to the order in which the steps or acts of the method are recited.

Where ranges are given herein, the invention provides embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. “About” in reference to a numerical value generally refers to a range of values that fall within ±10%, in some embodiments ±5%, in some embodiments ±1%, in some embodiments ±0.5% of the value unless otherwise stated or otherwise evident from the context. In any embodiment of the invention in which a numerical value is prefaced by “about”, the invention provides an embodiment in which the exact value is recited. In any embodiment of the invention in which a numerical value is not prefaced by “about”, the invention provides an embodiment in which the value is prefaced by “about”. Where the phrase “at least” precedes a series of numbers, it is to be understood that the phrase applies to each number in the list (it being understood that, depending on the context, 100% of a value may be an upper limit). It is also understood that any particular embodiment, feature, or aspect of the present invention may be explicitly excluded from any one or more of the claims. For example, any compound, compound combination, or therapeutic indication may be excluded.

TABLE 1
A
Total cell expansion from 1000 BFU-E cells:

| Dex | 0 h | 24 h | 48 h | 72 h | 120 h |
|---|---|---|---|---|---|
| − | 1,000 | 6,438 (±1,619) | 43,867 (±9.975) | 183,744 (±38,094) | 654,148 (±226,509) |
| + | 1,000 | 5,367 (±1,626) | 50,013 (±18,160) | 297,340 (±114,540) | 2,484,148 (±988,022) |

B
Total colony numbers from 1000 BFU-E cells:

| | Dex | 0 h | 24 h | 48 h | 72 h | 120 h |
|---|---|---|---|---|---|---|
| Single CFU-E | − | 73 (±26) | 2,054 (±935) | 16,300 (±1,132) | 74,118 (±14,250) | 12,356 (±4,788) |
| | + | 73 (±26) | 1,164 (±593) | 25,342 (±5,714) | 122,413 (±14,086) | 904,050 (±434,778) |
| Small BFU-E | − | 277 (±89) | 839 (±362) | 2,975 (±646) | 1,170 (±208) | n/d |
| | + | 277 (±89) | 1,568 (±604) | 6,730 (±515) | 15,350 (±3,838) | n/d |
| Large BFU-E | − | 241 (±79) | 86 (±56) | 28 (±30) | 10 (±12) | n/d |
| | + | 241 (±79) | 181 (±132) | 130 (±53) | 150 (±58) | n/d |

TABLE 2
A
Total cell expansion from 1000 “BFU-E” cells

| | Dex/DMOG | day 0 | day 1 | day 2 | day 3 | day 4 | day 5 |
|---|---|---|---|---|---|---|---|
| Liquid culture | — | 1,000 | 4,000 | 31,000 | 92,100 | 340,000 | 600,000 |
| | DMOG | 1,000 | 4,200 | 26,000 | 130,000 | 430,000 | 820,000 |
| | DEX | 1,000 | 3,900 | 30,000 | 200,000 | 1,200,000 | 3,500,000 |
| | DMOG + DEX | 1,000 | 4,100 | 37,000 | 230,000 | 1,300,000 | 5,200,000 |

| | Dex/DMOG | day 6 | day 7 | day 8 | day 9 | day 10 | day 11 |
|---|---|---|---|---|---|---|---|
| Liquid culture | — | 580,000 | 490,000 | 640,000 | | | |
| | DMOG | 890,000 | 1,200,000 | 1,200,000 | | | |
| | DEX | 7,200,000 | 14,000,000 | 20,000,000 | 25,000,000 | 25,000,000 | 20,000,000 |
| | DMOG + DEX | 12,000,000 | 49,000,000 | 80,000,000 | 120,000,000 | 180,000,000 | 120,000,000 |

B
Total colony numbers from 1,000 “BFU-E” cells

| | Dex/DMOG | day 0 | day 1 | day 2 | day 3 | day 4 | day 5 |
|---|---|---|---|---|---|---|---|
| CFU-E | — | 30 (±11) | 830 (±199) | 6,303 (±1,156) | 11,349 (±3,904) | 10,450 (±2,319) | 4,635 (±1,430) |
| 1 cluster | DMOG | 30 (±11) | 637 (±227) | 6,991 (±1,226) | 23,685 (±5,919) | 27,530 (±13,756) | 26,730 (±9,557) |
| | DEX | 30 (±11) | 424 (±271) | 11,468 (±1,088) | 60,699 (±22,369) | 138,600 (±24,546) | 261,550 (±98,160) |
| | DMOG + DEX | 30 (±11) | 135 (±157) | 8,661 (±2,882) | 79,425 (±9,324) | 299,875 (±171,779) | 336,100 (±332,679) |
| small BFU-E | — | 262 (±64) | 1,320 (±492) | 2,366 (±519) | 2,052 (±1,114) | | 826 (±185) |
| 5-20 clusters | DMOG | 262 (±64) | 1,284 (±683) | 3,232 (±1,416) | 4,399 (±2,039) | | 1,000 (±365) |
| | DEX | 262 (±64) | 1,766 (±695) | 5,771 (±1,625) | 8,334 (±2,489) | | 10,271 (±3,344) |
| | DMOG + DEX | 262 (±64) | 1,543 (±707) | 7,850 (±2,939) | 19,588 (±1,577) | | 37,525 (±6,331) |
| large BFU-E | — | 423 (±31) | 283 (±66) | 150 (±43) | 115 (±49) | | 0 |
| >20 clusters | DMOG | 423 (±31) | 428 (±72) | 217 (±33) | 156 (±69) | | 0 |
| | DEX | 423 (±31) | 463 (±61) | 482 (±110) | 400 (±108) | | 0 |
| | DMOG + DEX | 423 (±31) | 724 (±83) | 1,192 (±167) | 1,658 (±386) | | 84 (±96) |

| | Dex/DMOG | day 6 | day 7 | day 8 |
|---|---|---|---|---|
| CFU-E | — | 495 (±462) | | |
| 1 cluster | DMOG | 9,598 (±8,627) | | |
| | DEX | 249,160 (±55,172) | 176,000 (±15,556) | 66,000 (±56,003) |
| | DMOG + DEX | 1,623,500 (±220,823) | 1,906,667 (±228,160) | 1,547,500 (±350,018) |
| small BFU-E | — | | | |
| 5-20 clusters | DMOG | | | |
| | DEX | | | |
| | DMOG + DEX | | | |
| large BFU-E | — | | | |
| >20 clusters | DMOG | | | |
| | DEX | | | |
| | DMOG + DEX | | | |

TABLE S1
A Colonies per 1000 plated cells, scored day 3

| Magnetic depletion antibodies | Dex | CFU-E |
|---|---|---|
| Lin− (n = 7) | − | 485 (±77) |
| | + | 472 (±79) |
| Lin−, CD32/16− (n = 2) | − | 634 (±19) |
| | + | 560 (±245) |
| Lin−, CD32/16−, Sca-1− (n = 3) | − | 717 (±105) |
| | + | 791 (±151) |
| Lin−, CD32/16−, Sca-1−, CD41− (n = 5) | − | 734 (±271) |
| | + | 689 (±212) |
| Lin−, CD32/16−, Sca-1−, CD41−, CD34− (n = 4) | − | 486 (±170) |
| (FLEP cells) | + | 640 (±148) |

B Colonies per 1000 plated cells, scored day 8-9

| Magnetic depletion antibodies | Dex | late BFU-E | early BFU-E | CFU-G/M/Meg | GEMM |
|---|---|---|---|---|---|
| Lin− (n = 7) | − | 14 (±8) | 21 (±9) | 38 (±10) | 5 (±3) |
| | + | 53 (±13) | 39 (±12) | 25 (±5) | 7 (±4) |
| Lin−, CD32/16− (n = 2) | − | 12 (±3) | 6 (±2) | 10 (±1) | 3 (±2) |
| | + | 69 (±14) | 37 (±12) | 17 (±10) | 5 (±2) |
| Lin−, CD32/16−, Sca-1− (n = 3) | − | 17 (±4) | 10 (±7) | 5 (±3) | 0.3 (±0.3) |
| | + | 110 (±10) | 24 (±5) | 7 (±3) | 0.2 (±0.3) |
| Lin−, CD32/16−, Sca-1−, CD41− (n = 5) | − | 33 (±26) | 6 (±4) | 4 (±5) | 0.3 (±0.4) |
| | + | 142 (±77) | 10 (±4) | 9 (±12) | 0.3 (±0.4) |
| Lin−, CD32/16−, Sca-1−, CD41−, CD34− (n = 4) | − | 42 (±33) | 5 (±2) | 2 (±4) | 0.3 (±0.5) |
| (FLEP cells) | + | 141 (±84) | 23 (±17) | 4 (±6) | 0.3 (±0.5) |

TABLE S2
Colonies per 1000 plated cells

| Cell population | DEX/DMOG | CFU-E | small BFU-E | large BFU-E | CFU-G/M/Meg | GEMM |
|---|---|---|---|---|---|---|
| CD71 10% low | − | 110 (±58) | 194 (±40) | 66 (±57) | 21 (±19) | 0 |
| (n = 6) | DEX | 66 (±28) | 289 (±85)* | 222 (±84)* | 20 (±21) | 0 |
| CD71 20% high | − | 605 (±126) | 2 (±5) | 0 | 0 | 0 |
| (n = 9) | DEX | 565 (±120) | 17 (±31) | 0 | 0 | 0 |
| CD24a 10% low | − | n/d | n/d | n/d | n/d | n/d |
| (n = 6) | DEX | 96 (±23) | 336 (±27) | 178 (±28) | 19 (±6) | 0.8 (±2) |
| CD24a 20% high | − | n/d | n/d | n/d | n/d | n/d |
| (n = 6) | DEX | 454 (±19) | 15 (±2) | 0.4 (±2) | 0.3 (±0.3) | 0 |
| CD71/CD24a 10% low | − | 84 (±20) | 337 (±68) | 177 (±29) | 9 (±8) | 0 |
| (n = 4) | DEX | 33 (±10) | 251 (±40) | 412 (±23) | 7 (±2) | 0 |
| | DMOG | 21 (±11) | 243 (±30) | 164 (±23) | 5 (±6) | 0 |
| | DMOG + DEX | 7 (±2) | 83 (±13) | 516 (±63) | 6 (±1) | 0 |
| CD71 and CD24a 20% high | − | 518 | 0.7 | 0 | 0 | 0 |
| (n = 1) | + | 526 | 29 | 1 | 0 | 0 |

TABLE S3

| | | | | | | | Expression | | | | | |
|---|---|---|---|---|---|---|---|---|---|---|---|---|
| | | Whole | Whole | | number of | Expression | of mRNA | | Expression | | | |
| | Gene | Genome | Genome | | caCGTGgc | of mRNA in | in BFU-E | Expression | change | | | |
| | found in | rVista | rVista | | (MYC/HIF1a) | BFU-E after | after 4 h | level | log2 | | | |
| Name of | Whole | predicted | predicted c- | Gene | motifs | 4 h 0 nM | 100 nM | log2((100 nM | ((100 nM | | | |
| upregulated | Genome | HIF1 | myc | found in | −2000 to | DEX | DEX | DEX)* (0 nM | DEX)/(0 nM | GENE | | |
| Gene | rVista | target* | target* | WebMOTIFS | +200 bp | (RPKM) | (RPKM) | DEX)) | DEX))2 | ID | GENE NAME | Gene Ontology |
| Ddit4 | YES | YES | YES | YES | 0 | 2.39 | 20.23 | 5.59 | 3.08 | 74747 | DNA-damage- | molecular_function; |
| | | | | | | | | | | | inducible | cellular_component; |
| | | | | | | | | | | | transcript 4 | cytoplasm; |
| | | | | | | | | | | | | apoptosis; biological_process; |
| | | | | | | | | | | | | negative regulation |
| | | | | | | | | | | | | of signal transduction |
| Egln3 | YES | NO (but | — | YES | 0 | 2.05 | 16.47 | 5.08 | 3.00 | 112407 | EGL nine | iron ion |
| | | confirmed | | | | | | | | | homolog 3 | binding; nucleus; cytoplasm; |
| | | by | | | | | | | | | (C. elegans) | apoptosis; oxidoreductase |
| | | PubMed | | | | | | | | | | activity; oxidoreductase |
| | | ID: | | | | | | | | | | activity, acting on single |
| | | 15823097) | | | | | | | | | | donors with incorporation |
| | | | | | | | | | | | | of molecular oxygen, |
| | | | | | | | | | | | | incorporation of two |
| | | | | | | | | | | | | atoms of |
| | | | | | | | | | | | | oxygen; oxidoreductase |
| | | | | | | | | | | | | activity, acting on paired |
| | | | | | | | | | | | | donors, with incor |
| Zfp1 | YES | — | — | YES | 1 | 0.73 | 5.67 | 2.05 | 2.96 | 22640 | zinc finger | nucleic acid binding; DNA |
| | | | | | | | | | | | protein 1 | binding; intracellular; nucleus; |
| | | | | | | | | | | | | transcription; regulation |
| | | | | | | | | | | | | of transcription, DNA- |
| | | | | | | | | | | | | dependent; zinc ion |
| | | | | | | | | | | | | binding; metal ion binding |
| Arhgef3 | YES | — | YES | YES | 0 | 2.20 | 16.21 | 5.15 | 2.88 | 71704 | Rho guanine | molecular_function; guanyl- |
| | | | | | | | | | | | nucleotide | nucleotide exchange |
| | | | | | | | | | | | exchange factor | factor activity; Rho guanyl |
| | | | | | | | | | | | (GEF) 3 | nucleotide exchange |
| | | | | | | | | | | | | factor |
| | | | | | | | | | | | | activity; cellular_component; |
| | | | | | | | | | | | | intracellular; cytoplasm; |
| | | | | | | | | | | | | intracellular signaling |
| | | | | | | | | | | | | cascade; biological_process; |
| | | | | | | | | | | | | regulation of Rho |
| | | | | | | | | | | | | protein signal |
| | | | | | | | | | | | | transduction |
| 0610040J01Rik | YES | — | YES | YES | 2 | 2.06 | 14.77 | 4.93 | 2.84 | 76261 | RIKEN cDNA | |
| | | | | | | | | | | | 0610040J01 | |
| Socs1 | YES | — | — | YES | 0 | 5.08 | 34.03 | 7.43 | 2.74 | 12703 | suppressor of | regulation of protein |
| | | | | | | | | | | | cytokine | amino acid |
| | | | | | | | | | | | signaling 1 | phosphorylation; protein |
| | | | | | | | | | | | | binding; cellular_component; |
| | | | | | | | | | | | | intracellular signaling |
| | | | | | | | | | | | | cascade; JAK-STAT |
| | | | | | | | | | | | | cascade; negative |
| | | | | | | | | | | | | regulation of signal |
| | | | | | | | | | | | | transduction; kinase |
| | | | | | | | | | | | | inhibitor activity; cytokine |
| | | | | | | | | | | | | mediated signaling |
| | | | | | | | | | | | | pathway; modification- |
| | | | | | | | | | | | | depe |
| Acp5 | YES | — | YES | YES | 2 | 3.14 | 20.40 | 6.00 | 2.70 | 11433 | acid | acid phosphatase |
| | | | | | | | | | | | phosphatase 5, | activity; iron ion |
| | | | | | | | | | | | tartrate | binding; lysosome; immune |
| | | | | | | | | | | | resistant | response; dephosphorylation; |
| | | | | | | | | | | | | hydrolase |
| | | | | | | | | | | | | activity; response to |
| | | | | | | | | | | | | lipopolysaccharide; negative |
| | | | | | | | | | | | | regulation of |
| | | | | | | | | | | | | interleukin-1 beta |
| | | | | | | | | | | | | production; negative |
| | | | | | | | | | | | | regulation of interleukin- |
| | | | | | | | | | | | | 12 production; negative |
| | | | | | | | | | | | | regulati |
| Gsta4 | YES | — | — | YES | 0 | 1.49 | 9.00 | 3.74 | 2.60 | 14860 | glutathione S- | glutathione transferase |
| | | | | | | | | | | | transferase, | activity; cellular_component; |
| | | | | | | | | | | | alpha 4 | cytoplasm; metabolic |
| | | | | | | | | | | | | process |
| Maff | YES | YES | YES | YES | 0 | 1.02 | 6.11 | 2.65 | 2.58 | 17133 | v-maf | in utero embryonic |
| | | | | | | | | | | | musculoaponeurotic | development; DNA |
| | | | | | | | | | | | fibrosarcoma | binding; transcription |
| | | | | | | | | | | | oncogene | factor |
| | | | | | | | | | | | family, protein | activity; nucleus; regulation |
| | | | | | | | | | | | F (avian) | of transcription, DNA- |
| | | | | | | | | | | | | dependent; sequence- |
| | | | | | | | | | | | | specific DNA |
| | | | | | | | | | | | | binding; regulation of |
| | | | | | | | | | | | | epidermal cell |
| | | | | | | | | | | | | differentiation |
| Pfkfb4 | YES | NO (but | YES | YES | 0 | 2.22 | 12.25 | 4.76 | 2.47 | 270198 | 6- | nucleotide |
| | | confirmed | | | | | | | | | phosphofructo- | binding; catalytic |
| | | by | | | | | | | | | 2- | activity; 6-phosphofructo- |
| | | PubMed | | | | | | | | | kinase/fructose- | 2-kinase activity; fructose |
| | | ID: 17143338) | | | | | | | | | 2,6- | 2,6-bisphosphate 2- |
| | | | | | | | | | | | biphosphatase 4 | phosphatase activity; ATP |
| | | | | | | | | | | | | binding; fructose |
| | | | | | | | | | | | | metabolic |
| | | | | | | | | | | | | process; fructose 2,6- |
| | | | | | | | | | | | | bisphosphate metabolic |
| | | | | | | | | | | | | process; transferase |
| | | | | | | | | | | | | activity; hydrolase activity |
| Itgb3 | YES | — | YES | YES | 2 | 1.43 | 7.39 | 3.41 | 2.37 | 16416 | integrin beta 3 | receptor activity; integrin |
| | | | | | | | | | | | | binding; protein |
| | | | | | | | | | | | | binding; cell-substrate |
| | | | | | | | | | | | | junction assembly; cell |
| | | | | | | | | | | | | adhesion; cell-matrix |
| | | | | | | | | | | | | adhesion; integrin- |
| | | | | | | | | | | | | mediated signaling |
| | | | | | | | | | | | | pathway; integrin |
| | | | | | | | | | | | | complex; external side of |
| | | | | | | | | | | | | plasma |
| | | | | | | | | | | | | membrane; integral to |
| | | | | | | | | | | | | membrane; regulation of |
| | | | | | | | | | | | | cell migration |
| Per1 | YES | YES | YES | YES | 4 | 4.75 | 23.34 | 6.79 | 2.30 | 18626 | period homolog | signal transducer |
| | | | | | | | | | | | 1 (Drosophila) | activity; protein |
| | | | | | | | | | | | | binding; nucleus; cytoplasm; |
| | | | | | | | | | | | | transcription; regulation |
| | | | | | | | | | | | | of transcription, DNA- |
| | | | | | | | | | | | | dependent; signal |
| | | | | | | | | | | | | transduction; circadian |
| | | | | | | | | | | | | rhythm; response to light |
| | | | | | | | | | | | | stimulus; negative |
| | | | | | | | | | | | | regulation of gene- |
| | | | | | | | | | | | | specific transcription |
| | | | | | | | | | | | | from RNA polymerase II |
| Clca1 | YES | | — | YES | 3 | 1.80 | 7.54 | 3.76 | 2.07 | 12722 | chloride | intracellular calcium |
| | | | | | | | | | | | channel calcium | activated chloride |
| | | | | | | | | | | | activated 1 | channel activity; integral |
| | | | | | | | | | | | | to plasma |
| | | | | | | | | | | | | membrane; chloride |
| | | | | | | | | | | | | transport |
| Serpina3g | NO | n/a | n/a | NO | n/a | 8.65 | 36.06 | 8.28 | 2.06 | 20715 | serine (or | serine-type |
| | | | | | | | | | | | cysteine) | endopeptidase inhibitor |
| | | | | | | | | | | | peptidase | activity; cysteine-type |
| | | | | | | | | | | | inhibitor, clade | endopeptidase inhibitor |
| | | | | | | | | | | | A, member 3G | activity; cellular_component; |
| | | | | | | | | | | | | nucleus; cytoplasm; apoptosis; |
| | | | | | | | | | | | | immune |
| | | | | | | | | | | | | response; response to |
| | | | | | | | | | | | | cytokine |
| | | | | | | | | | | | | stimulus; response to |
| | | | | | | | | | | | | peptide hormone |
| | | | | | | | | | | | | stimulus |
| Ank | YES | NO (but | YES | YES | 1 | 11.69 | 48.60 | 9.15 | 2.06 | 11732 | progressive | integral to plasma |
| | | confirmed | | | | | | | | | ankylosis | membrane; transport; phosphate |
| | | by | | | | | | | | | | transport; phosphate |
| | | PubMed | | | | | | | | | | transmembrane |
| | | ID: 19419319) | | | | | | | | | | transporter |
| | | | | | | | | | | | | activity; integral to |
| | | | | | | | | | | | | membrane; outer |
| | | | | | | | | | | | | membrane; regulation of |
| | | | | | | | | | | | | bone |
| | | | | | | | | | | | | mineralization; inorganic |
| | | | | | | | | | | | | diphosphate |
| | | | | | | | | | | | | transmembrane |
| | | | | | | | | | | | | transporter activity |
| Frmd4a | YES | — | YES | YES | 0 | 7.05 | 28.77 | 7.66 | 2.03 | 209630 | FERM domain | binding; cytoplasm; cytoskeleton |
| | | | | | | | | | | | containing 4A | |
| Klf5 | YES | YES | YES | YES | 3 | 1.26 | 4.68 | 2.56 | 1.89 | 12224 | Kruppel-like | angiogenesis; nucleic acid |
| | | | | | | | | | | | factor 5 | binding; DNA |
| | | | | | | | | | | | | binding; transcription |
| | | | | | | | | | | | | factor activity; protein |
| | | | | | | | | | | | | binding; intracellular; nucleus; |
| | | | | | | | | | | | | regulation of |
| | | | | | | | | | | | | transcription, DNA- |
| | | | | | | | | | | | | dependent; zinc ion |
| | | | | | | | | | | | | binding; microvillus |
| | | | | | | | | | | | | assembly; positive |
| | | | | | | | | | | | | regulation of |
| | | | | | | | | | | | | transcription; metal ion |
| | | | | | | | | | | | | binding |
| Ncoa7 | YES | — | — | YES | 2 | 2.04 | 7.54 | 3.94 | 1.89 | 211329 | nuclear | nucleus; transcription; Cell |
| | | | | | | | | | | | receptor | wall macromolecule |
| | | | | | | | | | | | coactivator 7 | catabolic |
| | | | | | | | | | | | | process; regulation of |
| | | | | | | | | | | | | transcription |
| Hopx | YES | — | YES | YES | 2 | 3.77 | 13.32 | 5.65 | 1.82 | 74318 | HOP homeobox | negative regulation of |
| | | | | | | | | | | | | transcription from RNA |
| | | | | | | | | | | | | polymerase II |
| | | | | | | | | | | | | promoter; trophectodermal |
| | | | | | | | | | | | | cell differentiation; DNA |
| | | | | | | | | | | | | binding; transcription |
| | | | | | | | | | | | | factor activity; protein |
| | | | | | | | | | | | | binding; nucleus; cytoplasm; |
| | | | | | | | | | | | | regulation of |
| | | | | | | | | | | | | transcription, DNA- |
| | | | | | | | | | | | | dependent; multicellular |
| | | | | | | | | | | | | organismal devel |
| Plk3 | YES | — | YES | YES | 1 | 1.70 | 5.75 | 3.29 | 1.76 | 12795 | polo-like kinase | nucleotide |
| | | | | | | | | | | | 3 (Drosophila) | binding; protein kinase |
| | | | | | | | | | | | | activity; protein |
| | | | | | | | | | | | | serine/threonine kinase |
| | | | | | | | | | | | | activity; protein |
| | | | | | | | | | | | | binding; ATP |
| | | | | | | | | | | | | binding; protein amino |
| | | | | | | | | | | | | acid |
| | | | | | | | | | | | | phosphorylation; membrane; |
| | | | | | | | | | | | | transferase |
| | | | | | | | | | | | | activity; polo kinase |
| | | | | | | | | | | | | kinase activity |
| Clstn1 | YES | — | YES | YES | 0 | 21.47 | 70.51 | 10.56 | 1.72 | 65945 | calsyntenin 1 | calcium ion |
| | | | | | | | | | | | | binding; protein |
| | | | | | | | | | | | | binding; extracellular |
| | | | | | | | | | | | | region; nucleus; endoplasmic |
| | | | | | | | | | | | | reticulum; Golgi |
| | | | | | | | | | | | | apparatus; plasma |
| | | | | | | | | | | | | membrane; cellular |
| | | | | | | | | | | | | calcium ion |
| | | | | | | | | | | | | homeostasis; cell |
| | | | | | | | | | | | | adhesion; homophilic cell |
| | | | | | | | | | | | | adhesion; synaptic |
| | | | | | | | | | | | | transmission; integral to |
| | | | | | | | | | | | | membrane; cell |
| | | | | | | | | | | | | junction; cell pr |
| Fkbp5 | YES | YES | YES | YES | 4 | 22.79 | 74.76 | 10.73 | 1.71 | 14229 | FK506 binding | peptidyl-prolyl cis-trans |
| | | | | | | | | | | | protein 5 | isomerase |
| | | | | | | | | | | | | activity; binding; nucleus; |
| | | | | | | | | | | | | cytoplasm; protein folding |
| Bcl2 | YES | — | YES | YES | 1 | 2.00 | 6.45 | 3.69 | 1.69 | 12043 | B-cell | G1/S transition of mitotic |
| | | | | | | | | | | | leukemia/ | cell cycle; protein |
| | | | | | | | | | | | lymphoma 2 | phosphatase type 2A |
| | | | | | | | | | | | | complex; protein |
| | | | | | | | | | | | | polyubiquitination; cell |
| | | | | | | | | | | | | morphogenesis; response |
| | | | | | | | | | | | | to |
| | | | | | | | | | | | | acid; ossification; ovarian |
| | | | | | | | | | | | | follicle |
| | | | | | | | | | | | | development; metanephros |
| | | | | | | | | | | | | development; ureteric |
| | | | | | | | | | | | | bud |
| | | | | | | | | | | | | development; branching |
| | | | | | | | | | | | | involved in urete |
| Rcsd1 | YES | | — | YES | 0 | 2.53 | 8.11 | 4.36 | 1.68 | 226594 | RCSD domain | |
| | | | | | | | | | | | containing 1 | |
| Fam46a | NO | n/a | n/a | NO | n/a | 2.01 | 6.44 | 3.70 | 1.68 | 212943 | family with | |
| | | | | | | | | | | | sequence | |
| | | | | | | | | | | | similarity 46, | |
| | | | | | | | | | | | member A | |
| Egr1 | YES | YES | YES | YES | 0 | 5.94 | 18.06 | 6.75 | 1.60 | 13653 | early growth | negative regulation of |
| | | | | | | | | | | | response 1 | transcription from RNA |
| | | | | | | | | | | | | polymerase II |
| | | | | | | | | | | | | promoter; nucleic acid |
| | | | | | | | | | | | | binding; DNA |
| | | | | | | | | | | | | binding; transcription |
| | | | | | | | | | | | | factor |
| | | | | | | | | | | | | activity; intracellular; nucleus; |
| | | | | | | | | | | | | regulation of |
| | | | | | | | | | | | | transcription, DNA- |
| | | | | | | | | | | | | dependent; zinc ion |
| | | | | | | | | | | | | binding; response to |
| | | | | | | | | | | | | glucose |
| | | | | | | | | | | | | stimulus; transcriptio |
| Slc30a10 | YES | — | — | NO | n/a | 2.34 | 7.11 | 4.06 | 1.60 | 226781 | solute carrier | plasma |
| | | | | | | | | | | | family 30, | membrane; transport; ion |
| | | | | | | | | | | | member 10 | transport; cation |
| | | | | | | | | | | | | transport; zinc ion |
| | | | | | | | | | | | | transport; zinc ion |
| | | | | | | | | | | | | binding; cation |
| | | | | | | | | | | | | transmembrane |
| | | | | | | | | | | | | transporter |
| | | | | | | | | | | | | activity; integral to |
| | | | | | | | | | | | | membrane |
| Hp | YES | — | — | YES | 1 | 1.51 | 4.41 | 2.73 | 1.55 | 15439 | haptoglobin | catalytic activity; serine- |
| | | | | | | | | | | | | type endopeptidase |
| | | | | | | | | | | | | activity; extracellular |
| | | | | | | | | | | | | region; proteolysis; hemoglobin |
| | | | | | | | | | | | | binding |
| Cables1 | YES | YES | YES | YES | 1 | 1.72 | 4.90 | 3.07 | 1.51 | 63955 | CDK5 and Abl | G1/S transition of mitotic |
| | | | | | | | | | | | enzyme | cell cycle; protein |
| | | | | | | | | | | | substrate 1 | binding; nucleus; cytoplasm; |
| | | | | | | | | | | | | nervous system |
| | | | | | | | | | | | | development; cyclin- |
| | | | | | | | | | | | | dependent protein kinase |
| | | | | | | | | | | | | regulator activity; cell |
| | | | | | | | | | | | | projection; cell |
| | | | | | | | | | | | | division; regulation of cell |
| | | | | | | | | | | | | division; regulation of cell |
| | | | | | | | | | | | | cycle |
| Tph1 | YES | — | YES | YES | 0 | 1.93 | 5.51 | 3.41 | 1.51 | 21990 | tryptophan | monooxygenase |
| | | | | | | | | | | | hydroxylase 1 | activity; tryptophan 5- |
| | | | | | | | | | | | | monooxygenase |
| | | | | | | | | | | | | activity; iron ion |
| | | | | | | | | | | | | binding; metabolic |
| | | | | | | | | | | | | process; aromatic amino |
| | | | | | | | | | | | | acid family metabolic |
| | | | | | | | | | | | | process; oxidoreductase |
| | | | | | | | | | | | | activity; amino acid |
| | | | | | | | | | | | | binding; oxidoreductase |
| | | | | | | | | | | | | activity, acting on paired |
| | | | | | | | | | | | | donors, with |
| | | | | | | | | | | | | incorporation or r |
| Mt2 | YES | — | — | YES | 1 | 460.94 | 1306.42 | 19.20 | 1.50 | 17750 | metallothionein 2 | cellular zinc ion |
| | | | | | | | | | | | | homeostasis; nitric oxide |
| | | | | | | | | | | | | mediated signal |
| | | | | | | | | | | | | transduction; zinc ion |
| | | | | | | | | | | | | binding; detoxification of |
| | | | | | | | | | | | | copper ion; metal ion |
| | | | | | | | | | | | | binding |
| St3gal2 | YES | — | — | YES | 1 | 8.28 | 23.29 | 7.59 | 1.49 | 20444 | ST3 beta- | extracellular region; Golgi |
| | | | | | | | | | | | galactoside | apparatus; protein amino |
| | | | | | | | | | | | alpha-2,3- | acid |
| | | | | | | | | | | | sialyltransferase 2 | glycosylation; sialyltransferase |
| | | | | | | | | | | | | activity; membrane; integral |
| | | | | | | | | | | | | to |
| | | | | | | | | | | | | membrane; transferase |
| | | | | | | | | | | | | activity, transferring |
| | | | | | | | | | | | | glycosyl groups; integral |
| | | | | | | | | | | | | to Golgi membrane |
| Nhej1 | YES | — | YES | YES | 0 | 1.61 | 4.51 | 2.86 | 1.49 | 75570 | nonhomologous | DNA binding; protein |
| | | | | | | | | | | | end-joining | binding; nucleus; DNA |
| | | | | | | | | | | | factor 1 | repair; double-strand |
| | | | | | | | | | | | | break repair via |
| | | | | | | | | | | | | nonhomologous end |
| | | | | | | | | | | | | joining; response to DNA |
| | | | | | | | | | | | | damage |
| | | | | | | | | | | | | stimulus; response to |
| | | | | | | | | | | | | ionizing radiation; B cell |
| | | | | | | | | | | | | differentiation; T cell |
| | | | | | | | | | | | | differentiation |
| Lama5 | YES | — | YES | NO | n/a | 1.39 | 3.90 | 2.44 | 1.49 | 16776 | laminin, alpha 5 | branching involved in |
| | | | | | | | | | | | | ureteric bud |
| | | | | | | | | | | | | morphogenesis; morphogenesis |
| | | | | | | | | | | | | of a polarized |
| | | | | | | | | | | | | epithelium; neural crest |
| | | | | | | | | | | | | cell migration; hair follicle |
| | | | | | | | | | | | | development; receptor |
| | | | | | | | | | | | | binding; integrin |
| | | | | | | | | | | | | binding; protein |
| | | | | | | | | | | | | binding; extracellular |
| | | | | | | | | | | | | region; proteinaceous |
| | | | | | | | | | | | | extracellular |
| | | | | | | | | | | | | matrix; baseme |
| Wdr21 | YES | — | — | YES | 1 | 17.19 | 48.20 | 9.69 | 1.49 | 73828 | DDB1 and CUL4 | |
| | | | | | | | | | | | associated | |
| | | | | | | | | | | | factor 4 | |
| Pex11a | YES | — | — | YES | 3 | 2.10 | 5.87 | 3.62 | 1.49 | 18631 | peroxisomal | peroxisome; peroxisomal |
| | | | | | | | | | | | biogenesis | membrane; peroxisome |
| | | | | | | | | | | | factor 11 alpha | organization; integral to |
| | | | | | | | | | | | | membrane; peroxisome |
| | | | | | | | | | | | | fission; brown fat cell |
| | | | | | | | | | | | | differentiation |
| Gna14 | YES | — | YES | YES | 0 | 1.21 | 3.37 | 2.03 | 1.47 | 14675 | guanine | nucleotide |
| | | | | | | | | | | | nucleotide | binding; GTPase |
| | | | | | | | | | | | binding protein, | activity; signal transducer |
| | | | | | | | | | | | alpha 14 | activity; GTP |
| | | | | | | | | | | | | binding; heterotrimeric G- |
| | | | | | | | | | | | | protein complex; protein |
| | | | | | | | | | | | | amino acid ADP- |
| | | | | | | | | | | | | ribosylation; signal |
| | | | | | | | | | | | | transduction; G-protein |
| | | | | | | | | | | | | coupled receptor protein |
| | | | | | | | | | | | | signaling pathway; guanyl |
| | | | | | | | | | | | | nucleotide binding |
| Ampd3 | YES | — | YES | YES | 2 | 5.68 | 15.71 | 6.48 | 1.47 | 11717 | adenosine | AMP deaminase |
| | | | | | | | | | | | monophosphate | activity; purine base |
| | | | | | | | | | | | deaminase 3 | metabolic |
| | | | | | | | | | | | | process; nucleotide |
| | | | | | | | | | | | | metabolic process; purine |
| | | | | | | | | | | | | ribonucleoside |
| | | | | | | | | | | | | monophosphate |
| | | | | | | | | | | | | biosynthetic |
| | | | | | | | | | | | | process; hydrolase |
| | | | | | | | | | | | | activity; hydrolase |
| | | | | | | | | | | | | activity, acting on carbon- |
| | | | | | | | | | | | | nitrogen (but not |
| | | | | | | | | | | | | peptide) bonds, in cyclic |
| | | | | | | | | | | | | amidines |
| Fam46c | NO | n/a | n/a | NO | n/a | 5.12 | 13.90 | 6.15 | 1.44 | 74645 | family with | |
| | | | | | | | | | | | sequence | |
| | | | | | | | | | | | similarity 46, | |
| | | | | | | | | | | | member C | |
| Tsc22d3 | YES | — | YES | YES | 1 | 7.39 | 19.83 | 7.20 | 1.42 | 14605 | TSC22 domain | transcription factor |
| | | | | | | | | | | | family, member 3 | activity; regulation of |
| | | | | | | | | | | | | transcription, DNA- |
| | | | | | | | | | | | | dependent; anti- |
| | | | | | | | | | | | | apoptosis; response to |
| | | | | | | | | | | | | osmotic stress |
| Tns1 | NO | n/a | n/a | NO | n/a | 7.48 | 20.01 | 7.22 | 1.42 | 21961 | tensin 1 | actin binding; focal |
| | | | | | | | | | | | | adhesion; cell-substrate |
| | | | | | | | | | | | | junction assembly; cell |
| | | | | | | | | | | | | migration |
| Fam83g | YES | YES | YES | YES | 3 | 3.25 | 8.67 | 4.82 | 1.41 | 69640 | family with | |
| | | | | | | | | | | | sequence | |
| | | | | | | | | | | | similarity 83, | |
| | | | | | | | | | | | member G | |
| Syne1 | YES | — | YES | YES | 2 | 2.79 | 7.36 | 4.36 | 1.40 | 64009 | synaptic | actin binding; protein |
| | | | | | | | | | | | nuclear | binding; nuclear |
| | | | | | | | | | | | envelope 1 | envelope; |
| | | | | | | | | | | | | sarcomere; establishment |
| | | | | | | | | | | | | of nucleus |
| | | | | | | | | | | | | localization |
| Hif3a | YES | YES | YES | YES | 1 | 39.00 | 101.46 | 11.95 | 1.38 | 53417 | hypoxia | response to hypoxia; DNA |
| | | | | | | | | | | | inducible factor | binding; transcription |
| | | | | | | | | | | | 3, alpha | factor activity; signal |
| | | | | | | | | | | | subunit | transducer |
| | | | | | | | | | | | | activity; nucleus; cytoplasm; |
| | | | | | | | | | | | | regulation of |
| | | | | | | | | | | | | transcription, DNA- |
| | | | | | | | | | | | | dependent; transcription |
| | | | | | | | | | | | | from RNA polymerase II |
| | | | | | | | | | | | | promoter; signal |
| | | | | | | | | | | | | transduction; transcription |
| | | | | | | | | | | | | regulator activity |
| Klf13 | YES | — | YES | YES | 1 | 11.46 | 29.57 | 8.41 | 1.37 | 50794 | Kruppel-like | nucleic acid binding; DNA |
| | | | | | | | | | | | factor 13 | binding; RNA polymerase |
| | | | | | | | | | | | | II transcription factor |
| | | | | | | | | | | | | activity; intracellular; nucleus; |
| | | | | | | | | | | | | transcription from |
| | | | | | | | | | | | | RNA polymerase II |
| | | | | | | | | | | | | promoter; zinc ion |
| | | | | | | | | | | | | binding; regulation of |
| | | | | | | | | | | | | transcription; positive |
| | | | | | | | | | | | | regulation of |
| | | | | | | | | | | | | transcription from RNA |
| | | | | | | | | | | | | polymerase II p |
| Nfkbia | YES | | YES | YES | 0 | 3.02 | 7.75 | 4.55 | 1.36 | 18035 | nuclear factor | protein import into |
| | | | | | | | | | | | of kappa light | nucleus, |
| | | | | | | | | | | | polypeptide | translocation; cytoplasm; |
| | | | | | | | | | | | gene enhancer | cytosol; lipopolysaccharide- |
| | | | | | | | | | | | in B-cells | mediated signaling |
| | | | | | | | | | | | inhibitor, alpha | pathway; negative |
| | | | | | | | | | | | | regulation of NF-kappaB |
| | | | | | | | | | | | | transcription factor |
| | | | | | | | | | | | | activity; response to |
| | | | | | | | | | | | | muramyl |
| | | | | | | | | | | | | dipeptide; response to |
| | | | | | | | | | | | | lipopolysaccharide; toll- |
| | | | | | | | | | | | | like receptor 4 sig |
| Zfp36l2 | YES | YES | YES | YES | 1 | 64.10 | 163.74 | 13.36 | 1.35 | 12193 | zinc finger | nuclear-transcribed |
| | | | | | | | | | | | protein 36, C3H | mRNA catabolic process, |
| | | | | | | | | | | | type-like 2 | deadenylation-dependent |
| | | | | | | | | | | | | decay; nucleic acid |
| | | | | | | | | | | | | binding; DNA binding; RNA |
| | | | | | | | | | | | | binding; nucleus; cytoplasm; |
| | | | | | | | | | | | | zinc ion |
| | | | | | | | | | | | | binding; regulation of |
| | | | | | | | | | | | | mRNA stability; metal ion |
| | | | | | | | | | | | | binding |
| Aqp3 | YES | — | YES | YES | 2 | 2.75 | 6.98 | 4.27 | 1.34 | 11828 | aquaporin 3 | transporter |
| | | | | | | | | | | | | activity; integral to |
| | | | | | | | | | | | | plasma membrane; water |
| | | | | | | | | | | | | transport; water channel |
| | | | | | | | | | | | | activity; integral to |
| | | | | | | | | | | | | membrane; basolateral |
| | | | | | | | | | | | | plasma membrane; pore |
| | | | | | | | | | | | | complex; transmembrane |
| | | | | | | | | | | | | transport |
| Ccnd1 | YES | YES | YES | YES | 0 | 2.32 | 5.86 | 3.76 | 1.34 | 12443 | cyclin D1 | cyclin-dependent protein |
| | | | | | | | | | | | | kinase holoenzyme |
| | | | | | | | | | | | | complex; re-entry into |
| | | | | | | | | | | | | mitotic cell cycle; protein |
| | | | | | | | | | | | | kinase |
| | | | | | | | | | | | | activity; nucleus; nucleoplasm; |
| | | | | | | | | | | | | cytosol; protein |
| | | | | | | | | | | | | amino acid |
| | | | | | | | | | | | | phosphorylation; lactation; |
| | | | | | | | | | | | | cyclin-dependent protein |
| | | | | | | | | | | | | kinase regulator |
| | | | | | | | | | | | | activity; negative |
| | | | | | | | | | | | | regulation of |
| Zbtb16 | YES | YES | YES | NO | n/a | 2.49 | 6.14 | 3.93 | 1.30 | 235320 | zinc finger and | skeletal system |
| | | | | | | | | | | | BTB domain | development; DNA |
| | | | | | | | | | | | containing 16 | binding; protein |
| | | | | | | | | | | | | binding; nucleus; zinc ion |
| | | | | | | | | | | | | binding; negative |
| | | | | | | | | | | | | regulation of cell |
| | | | | | | | | | | | | proliferation; embryonic |
| | | | | | | | | | | | | pattern |
| | | | | | | | | | | | | specification; anterior/posterior |
| | | | | | | | | | | | | pattern |
| | | | | | | | | | | | | formation; specific |
| | | | | | | | | | | | | transcriptional repressor |
| | | | | | | | | | | | | activity; transcriptional |
| | | | | | | | | | | | | rep |
| Tle4 | YES | YES | YES | YES | 2 | 6.67 | 16.27 | 6.76 | 1.29 | 21888 | transducin-like | negative regulation of |
| | | | | | | | | | | | enhancer of | transcription from RNA |
| | | | | | | | | | | | split 4, | polymerase II |
| | | | | | | | | | | | homolog of | promoter; chromatin |
| | | | | | | | | | | | Drosophila | binding; RNA polymerase |
| | | | | | | | | | | | E(spl) | II transcription factor |
| | | | | | | | | | | | | activity, enhancer |
| | | | | | | | | | | | | binding; transcription |
| | | | | | | | | | | | | corepressor |
| | | | | | | | | | | | | activity; protein |
| | | | | | | | | | | | | binding; nucleus; Wnt |
| | | | | | | | | | | | | receptor signaling |
| | | | | | | | | | | | | pathway |
| Fes | YES | YES | YES | YES | 0 | 1.46 | 3.48 | 2.34 | 1.26 | 14159 | feline sarcoma | nucleotide |
| | | | | | | | | | | | oncogene | binding; protein kinase |
| | | | | | | | | | | | | activity; protein tyrosine |
| | | | | | | | | | | | | kinase activity; non- |
| | | | | | | | | | | | | membrane spanning |
| | | | | | | | | | | | | protein tyrosine kinase |
| | | | | | | | | | | | | activity; protein |
| | | | | | | | | | | | | binding; ATP |
| | | | | | | | | | | | | binding; cytosol; protein |
| | | | | | | | | | | | | amino acid |
| | | | | | | | | | | | | phosphorylation; transferase |
| | | | | | | | | | | | | activity |
| Rps6ka1 | YES | — | YES | YES | 0 | 16.31 | 39.02 | 9.31 | 1.26 | 20111 | ribosomal | nucleotide |
| | | | | | | | | | | | protein S6 | binding; magnesium ion |
| | | | | | | | | | | | kinase | binding; protein kinase |
| | | | | | | | | | | | polypeptide 1 | activity; protein |
| | | | | | | | | | | | | serine/threonine kinase |
| | | | | | | | | | | | | activity; ATP |
| | | | | | | | | | | | | binding; spindle; ribosome; |
| | | | | | | | | | | | | protein amino add |
| | | | | | | | | | | | | phosphorylation; protein |
| | | | | | | | | | | | | kinase |
| | | | | | | | | | | | | cascade; transferase |
| | | | | | | | | | | | | activity |
| Gng13 | YES | — | YES | YES | 1 | 2.43 | 5.76 | 3.81 | 1.25 | 64337 | guanine | GTPase activity; signal |
| | | | | | | | | | | | nucleotide | transducer |
| | | | | | | | | | | | binding protein | activity; heterotrimeric G- |
| | | | | | | | | | | | (G protein), | protein complex; plasma |
| | | | | | | | | | | | gamma 13 | membrane; signal |
| | | | | | | | | | | | | transduction; G-protein |
| | | | | | | | | | | | | coupled receptor protein |
| | | | | | | | | | | | | signaling |
| | | | | | | | | | | | | pathway; activation of |
| | | | | | | | | | | | | phospholipase C activity |
| | | | | | | | | | | | | by G-protein coupled |
| | | | | | | | | | | | | receptor protein signaling |
| | | | | | | | | | | | | pat |
| Rapgef2 | NO | n/a | n/a | NO | n/a | 1.38 | 3.28 | 2.18 | 1.25 | 76089 | Rap guanine | guanyl-nucleotide |
| | | | | | | | | | | | nucleotide | exchange factor |
| | | | | | | | | | | | exchange factor | activity; protein |
| | | | | | | | | | | | (GEF) 2 | binding; intracellular; plasma |
| | | | | | | | | | | | | membrane; signal |
| | | | | | | | | | | | | transduction; small |
| | | | | | | | | | | | | GTPase mediated signal |
| | | | | | | | | | | | | transduction |
| Polr3gl | YES | — | YES | YES | 3 | 2.51 | 5.93 | 3.89 | 1.24 | 69870 | polymerase | |
| | | | | | | | | | | | (RNA) III (DNA | |
| | | | | | | | | | | | directed) | |
| Clcn2 | YES | YES | YES | YES | 0 | 3.74 | 8.78 | 5.04 | 1.23 | 12724 | chloride | ion channel |
| | | | | | | | | | | | channel 2 | activity; voltage-gated ion |
| | | | | | | | | | | | | channel activity; voltage- |
| | | | | | | | | | | | | gated chloride channel |
| | | | | | | | | | | | | activity; transport; ion |
| | | | | | | | | | | | | transport; chloride |
| | | | | | | | | | | | | transport; membrane; integral |
| | | | | | | | | | | | | to |
| | | | | | | | | | | | | membrane; chloride ion |
| | | | | | | | | | | | | binding; chloride channel |
| | | | | | | | | | | | | complex; transmembrane |
| | | | | | | | | | | | | transport; cell differentia |
| Man2a2 | YES | — | | YES | 2 | 15.30 | 35.22 | 9.07 | 1.20 | 140481 | mannosidase 2, | mannosidase |
| | | | | | | | | | | | alpha 2 | activity; hydrolase |
| | | | | | | | | | | | | activity, hydrolyzing N- |
| | | | | | | | | | | | | glycosyl compounds |
| Ier3 | YES | YES | YES | YES | 1 | 2.46 | 5.65 | 3.80 | 1.20 | 15937 | immediate | membrane; integral to |
| | | | | | | | | | | | early response | membrane |
| Clint1 | YES | — | YES | NO | n/a | 127.14 | 285.78 | 15.15 | 1.17 | 216705 | clathrin | protein |
| | | | | | | | | | | | interactor 1 | binding; cytoplasm; endocytosis; |
| | | | | | | | | | | | | lipid |
| | | | | | | | | | | | | binding; membrane; cytoplasmic |
| | | | | | | | | | | | | vesicle |
| Klf9 | YES | YES | YES | YES | 1 | 3.59 | 8.07 | 4.86 | 1.17 | 16601 | Kruppel-like | nucleic acid binding; DNA |
| | | | | | | | | | | | factor 9 | binding; intracellular; nucleus; |
| | | | | | | | | | | | | transcription; embryo |
| | | | | | | | | | | | | implantation; zinc ion |
| | | | | | | | | | | | | binding; transcription |
| | | | | | | | | | | | | regulator |
| | | | | | | | | | | | | activity; regulation of |
| | | | | | | | | | | | | transcription; metal ion |
| | | | | | | | | | | | | binding; progesterone |
| | | | | | | | | | | | | receptor signaling |
| | | | | | | | | | | | | pathway |
| Rab3il1 | YES | YES | YES | YES | 3 | 2.20 | 4.94 | 3.44 | 1.17 | 74760 | RAB3A | guanyl-nucleotide |
| | | | | | | | | | | | interacting | exchange factor |
| | | | | | | | | | | | protein (rabin3) | activity; protein |
| | | | | | | | | | | | like 1 | binding; Rab guanyl- |
| | | | | | | | | | | | | nucleotide exchange |
| | | | | | | | | | | | | factor activity |
| Txnip | YES | NO (but | YES | YES | 1 | 11.19 | 24.97 | 8.13 | 1.16 | 56338 | thioredoxin | negative regulation of |
| | | confirmed | | | | | | | | | interacting | transcription from RNA |
| | | by | | | | | | | | | protein | polymerase II |
| | | PubMed | | | | | | | | | | promoter; enzyme |
| | | ID: | | | | | | | | | | inhibitor activity; protein |
| | | 18376310) | | | | | | | | | | binding; cytoplasm; protein |
| | | | | | | | | | | | | import into |
| | | | | | | | | | | | | nucleus; response to |
| | | | | | | | | | | | | oxidative stress; cell |
| | | | | | | | | | | | | cycle; platelet-derived |
| | | | | | | | | | | | | growth factor receptor |
| | | | | | | | | | | | | signaling pathway |
| Slc44a2 | YES | YES | YES | YES | 1 | 16.21 | 35.91 | 9.19 | 1.15 | 68682 | solute carrier | transport; membrane; integral |
| | | | | | | | | | | | family 44, | to membrane |
| | | | | | | | | | | | member 2 | |
| 38413 | YES | — | YES | YES | 1 | 3.72 | 8.25 | 4.94 | 1.15 | 320253 | membrane- | endosome; endocytosis; zinc |
| | | | | | | | | | | | associated ring | ion |
| | | | | | | | | | | | finger (C3HC4) 3 | binding; membrane; integral |
| | | | | | | | | | | | | to membrane; ligase |
| | | | | | | | | | | | | activity; modification- |
| | | | | | | | | | | | | dependent protein |
| | | | | | | | | | | | | catabolic |
| | | | | | | | | | | | | process; cytoplasmic |
| | | | | | | | | | | | | vesicle; metal ion binding |
| Cd59a | YES | — | — | YES | 1 | 1.44 | 3.15 | 2.18 | 1.13 | 12509 | CD59a antigen | plasma |
| | | | | | | | | | | | | membrane; external side |
| | | | | | | | | | | | | of plasma |
| | | | | | | | | | | | | membrane; anchored to |
| | | | | | | | | | | | | membrane |
| Atp2b4 | YES | — | — | YES | 0 | 20.11 | 43.89 | 9.79 | 1.13 | 381290 | ATPase, Ca++ | nucleotide binding; ATP |
| | | | | | | | | | | | transporting | binding; integral to |
| | | | | | | | | | | | plasma | plasma |
| | | | | | | | | | | | membrane 4 | membrane; transport; ion |
| | | | | | | | | | | | | transport; calcium ion |
| | | | | | | | | | | | | transport; integral to |
| | | | | | | | | | | | | membrane; hydrolase |
| | | | | | | | | | | | | activity |
| Cyth3 | YES | — | — | YES | 1 | 34.55 | 75.31 | 11.35 | 1.12 | 19159 | cytohesin 3 | ruffle; guanyl-nucleotide |
| | | | | | | | | | | | | exchange factor |
| | | | | | | | | | | | | activity; ARF guanyl- |
| | | | | | | | | | | | | nucleotide exchange |
| | | | | | | | | | | | | factor activity; protein |
| | | | | | | | | | | | | binding; phosphatidylinositol- |
| | | | | | | | | | | | | 3,4,5-trisphosphate |
| | | | | | | | | | | | | binding; intracellular; cytoplasm; |
| | | | | | | | | | | | | plasma |
| | | | | | | | | | | | | membrane; regulation of |
| | | | | | | | | | | | | ARF protein signal |
| | | | | | | | | | | | | transduction; positiv |
| Trib2 | YES | — | YES | YES | 1 | 2.81 | 6.12 | 4.11 | 1.12 | 217410 | tribbles | protein kinase |
| | | | | | | | | | | | homolog 2 | activity; protein kinase |
| | | | | | | | | | | | (Drosophila) | inhibitor activity; protein |
| | | | | | | | | | | | | binding; ATP |
| | | | | | | | | | | | | binding; cytoplasm; |
| | | | | | | | | | | | | cytoskeleton; |
| | | | | | | | | | | | | protein amino acid |
| | | | | | | | | | | | | phosphorylation |
| Nrgn | YES | — | YES | YES | 0 | 9.78 | 21.01 | 7.68 | 1.10 | 64011 | neurogranin | calmodulin |
| | | | | | | | | | | | | binding; cytoplasm; protein |
| | | | | | | | | | | | | kinase cascade; cell |
| | | | | | | | | | | | | junction; cell |
| | | | | | | | | | | | | projection; synapse |
| Pld6 | YES | — | — | NO | n/a | 1.70 | 3.65 | 2.64 | 1.10 | 194908 | phospholipase | catalytic |
| | | | | | | | | | | | D family, | activity; phospholipase D |
| | | | | | | | | | | | member 6 | activity; metabolic |
| | | | | | | | | | | | | process; membrane; integral |
| | | | | | | | | | | | | to |
| | | | | | | | | | | | | membrane; hydrolase |
| | | | | | | | | | | | | activity; NAPE-specific |
| | | | | | | | | | | | | phospholipase D activity |
| Mobkl2c | YES | — | YES | YES | 0 | 4.07 | 8.65 | 5.14 | 1.09 | 100465 | MOB1, Mps One | protein binding; zinc ion |
| | | | | | | | | | | | Binder kinase | binding; kinase |
| | | | | | | | | | | | activator-like | activity; metal ion binding |
| | | | | | | | | | | | 2C (yeast) | |
| E430018J23Rik | YES | — | — | YES | 0 | 1.42 | 3.02 | 2.10 | 1.09 | 101604 | RIKEN cDNA | nucleus; transcription; zinc |
| | | | | | | | | | | | E430018J23 | ion binding; metal ion |
| | | | | | | | | | | | gene | binding |
| Spry1 | YES | NO (but | — | YES | 0 | 4.42 | 9.31 | 5.36 | 1.08 | 24063 | sprouty | metanephros |
| | | confirmed | | | | | | | | | homolog 1 | development; ureteric |
| | | by | | | | | | | | | (Drosophila) | bud |
| | | PubMed | | | | | | | | | | development; induction of |
| | | ID: | | | | | | | | | | an organ; protein |
| | | 20054616) | | | | | | | | | | binding; cytoplasm; multicellular |
| | | | | | | | | | | | | organismal |
| | | | | | | | | | | | | development; negative |
| | | | | | | | | | | | | regulation of cell |
| | | | | | | | | | | | | proliferation; regulation of |
| | | | | | | | | | | | | signal |
| | | | | | | | | | | | | transduction; membrane; |
| | | | | | | | | | | | | negative regulation of |
| | | | | | | | | | | | | Ras GTPase ac |
| Fkbp11 | YES | — | — | YES | 0 | 21.16 | 44.44 | 9.88 | 1.07 | 66120 | FK506 binding | peptidyl-prolyl cis-trans |
| | | | | | | | | | | | protein 11 | isomerase |
| | | | | | | | | | | | | activity; protein |
| | | | | | | | | | | | | folding; membrane; integral |
| | | | | | | | | | | | | to membrane |
| Kit | YES | — | YES | YES | 5 | 46.21 | 97.02 | 12.13 | 1.07 | 16590 | kit oncogene | nucleotide |
| | | | | | | | | | | | | binding; myeloid |
| | | | | | | | | | | | | progenitor cell |
| | | | | | | | | | | | | differentiation; lymphoid |
| | | | | | | | | | | | | progenitor cell |
| | | | | | | | | | | | | differentiation; myeloid |
| | | | | | | | | | | | | leukocyte |
| | | | | | | | | | | | | differentiation; protein |
| | | | | | | | | | | | | kinase activity; protein |
| | | | | | | | | | | | | tyrosine kinase |
| | | | | | | | | | | | | activity; transmembrane |
| | | | | | | | | | | | | receptor protein tyrosine |
| | | | | | | | | | | | | kinase activity; receptor |
| Fam55c | NO | n/a | n/a | NO | n/a | 5.35 | 11.20 | 5.91 | 1.07 | 385658 | family with | |
| | | | | | | | | | | | sequence | |
| | | | | | | | | | | | similarity 55, | |
| | | | | | | | | | | | member C | |
| Lrfn1 | YES | — | YES | YES | 1 | 1.62 | 3.37 | 2.45 | 1.05 | 80749 | leucine rich | protein |
| | | | | | | | | | | | repeat and | binding; membrane; integral |
| | | | | | | | | | | | fibronectin type | to membrane; cell |
| | | | | | | | | | | | III domain | junction; synapse |
| | | | | | | | | | | | containing 1 | |
| Ier2 | YES | — | YES | YES | 2 | 22.77 | 47.27 | 10.07 | 1.05 | 15936 | immediate | molecular_function; cytoplasm; |
| | | | | | | | | | | | early response 2 | biological_process |
| Wdr60 | YES | — | — | YES | 1 | 3.93 | 8.14 | 5.00 | 1.05 | 217935 | WD repeat | |
| | | | | | | | | | | | domain 60 | |
| Glul | YES | — | — | YES | 1 | 53.96 | 110.54 | 12.54 | 1.03 | 14645 | glutamate- | nucleotide |
| | | | | | | | | | | | ammonia ligase | binding; catalytic |
| | | | | | | | | | | | (glutamine | activity; glutamate- |
| | | | | | | | | | | | synthetase) | ammonia ligase |
| | | | | | | | | | | | | activity; ATP |
| | | | | | | | | | | | | binding; intracellular; cytoplasm; |
| | | | | | | | | | | | | mitochondrion; glutamine |
| | | | | | | | | | | | | biosynthetic |
| | | | | | | | | | | | | process; nitrogen |
| | | | | | | | | | | | | compound metabolic |
| | | | | | | | | | | | | process; response to |
| | | | | | | | | | | | | glucose stimulus |
| Nags | YES | — | — | YES | 1 | 1.42 | 2.88 | 2.04 | 1.02 | 217214 | N- | urea |
| | | | | | | | | | | | acetylglutamate | cycle; acetylglutamate |
| | | | | | | | | | | | synthase | kinase activity; acetyl- |
| | | | | | | | | | | | | CoA:L-glutamate N- |
| | | | | | | | | | | | | acetyltransferase |
| | | | | | | | | | | | | activity; mitochondrion; arginine |
| | | | | | | | | | | | | biosynthetic |
| | | | | | | | | | | | | process; glutamate |
| | | | | | | | | | | | | metabolic |
| | | | | | | | | | | | | process; acyltransferase |
| | | | | | | | | | | | | activity; cellular amino |
| | | | | | | | | | | | | acid biosynthetic process |
| Gm129 | YES | YES | YES | NO | n/a | 1.56 | 3.14 | 2.29 | 1.01 | 229599 | predicted gene | |
| | | | | | | | | | | | 129 | |

We claim:

1. A method of expanding BFU-E cells comprising contacting one or more BFU-E cells with a hypoxia inducible factor 1 (HIF-1) activator and a glucocorticoid receptor (GR) agonist.

2. The method of claim 1, wherein the HIF-1 activator is a prolyl hydroxylase inhibitor (PHI).

3. The method of claim 1, wherein the method comprises culturing BFU-E in medium containing a HIF-1a activator and a glucocorticoid receptor (GR) agonist.

4. The method of claim 1, wherein the BFU-E cells are human cells.

5. The method of claim 1, wherein the method comprises administering a HIF-1 activator and a glucocorticoid receptor (GR) agonist to a subject.

6. The method of claim 5, wherein the subject has anemia.

7. The method of claim 5, wherein the subject has an Epo-resistant anemia.

8. The method of claim 5, wherein the subject is human.

9. A method of expanding BFU-E cells in vitro comprising culturing or more BFU-E cells in media comprising a HIF-1 activator, a GR agonist, or both.

10. The method of claim 9, wherein BFU-E cells are obtained from a starting population comprising fetal liver cells, fetal spleen cells, peripheral blood cells, bone marrow cells, or umbilical cord blood cells.

11. The method of claim 9, wherein BFU-Es are obtained by in vitro culture and differentiation of hematopoietic stem cells (HSCs).

12. The method of claim 9, wherein BFU-Es are obtained from human CD34+ cells.

13. The method of claim 9, wherein the method results in an increased number of CFU-Es.

14. The method of claim 9, wherein the method results in an increased number of mature, functional RBCs.

15. The method of claim 9, wherein the media contains a HIF-1 activator and a GR agonist.

16. The method of claim 9, wherein the HIF-1 activator is a PHI.

17. The method of claim 9, wherein the BFU-E cells are human BFU-E cells.

18. The method of claim 9, further comprising administering at least some expanded cells to a subject in need thereof.

19. A method of treating a subject suffering from or at risk of anemia, the method comprising administering a HIF-1 activator and a GR agonist to the subject.

20. The method of claim 19, wherein the HIF-1 activator is a PHI.

21. The method of claim 19, wherein the anemia is anemia of chronic disease, anemia associated with chemotherapy, anemia associated with renal disease, or anemia associated with infection.

22. The method of claim 19, wherein the subject is expected to undergo surgery within 4 weeks of administration of the HIF-1 activator and the GR agonist.

23. The method of claim 19, wherein the subject has experienced acute or chronic blood loss.

24. The method of claim 19, wherein the anemia results at least in part from hemolysis.

25. The method of claim 19, wherein the anemia is a bone marrow failure syndrome.

26. The method of claim 19, wherein the anemia is Diamond-Blackfan syndrome.

27. The method of claim 19, wherein the GR agonist is a glucocorticoid.

28. The method of claim 19, wherein the subject is human.

29. A method of treating a subject suffering from or at risk of an Epo-resistant anemia comprising administering a HIF-1 activator to the subject.

30. The method of claim 29, wherein the method comprises administering a GR agonist in combination with the HIF-1 activator.

31. The method of claim 29, wherein the Epo-resistant anemia is anemia of chronic disease, anemia associated with chemotherapy, anemia associated with renal disease, or anemia associated with infection.

32. The method of claim 29, wherein the Epo-resistant anemia is a bone marrow failure syndrome.

33. The method of claim 29, wherein the Epo-resistant anemia is Diamond-Blackfan syndrome.

34. The method of claim 29, wherein the HIF-1 activator is a PHI.

35. The method of claim 29, wherein the GR agonist is a glucocorticoid.

36. The method of claim 29, wherein the subject is human.

37. A method of treating a subject suffering from an Epo-resistant anemia, wherein the Epo-resistant anemia is an anemia that is not currently treated with a GR agonist, the method comprising administering to the subject a compound that enhances survival or self-renewal of BFU-E cells.

38. The method of claim 37, wherein the compound that enhances survival or self-renewal of BFU-E cells is a HIF-1 activator.

39. The method of claim 37, wherein the compound that enhances survival or self-renewal of BFU-E cells is GR agonist.

40. The method of claim 37, wherein the method comprises administering a HIF-1 activator and a GR agonist to the subject.

41. A composition comprising a HIF-1 activator and a GR agonist.

42. The composition of claim 41, wherein the HIF-1 activator is a PHI.

43. The composition of claim 41, wherein the GR agonist is a glucocorticoid.

44. The composition of claim 41, wherein the HIF-1 activator is a PHI and the GR agonist is a glucocorticoid.

45. The composition of claim 41, wherein the HIF-1 activator is a PHI and the GR agonist is a synthetic glucocorticoid.

46. A purified cell population, wherein at least 75% of the cells are BFU-E cells.

47. The purified cell population of claim 46, wherein the population contains no more than 2% CFU-G/M/Mk cells.

48. The purified cell population of claim 46, wherein the population contains no more than 1% GEMM cells.

49. A purified cell population, wherein at least 50% of the cells are CFU-E cells.

50. A method of purifying BFU-E cells from a population of cells that comprises one or more BFU-E cells, the method comprising steps of: (a) depleting the population of cells that are positive for Ter119, CD16, CD32, Sca-1, and/or CD41; and (b) selecting cells that are (i) c-kit positive and (ii) CD71^{10% low} or CD24a^{10% low}.

51. The method of claim 50, wherein step (b) comprises selecting cells that are CD71^{10% low} and CD24a^{10% low}.

52. The method of claim 50, wherein the cells are selected from the group consisting of bone marrow cells, umbilical cord blood cells, peripheral blood cells, and fetal liver cells.

53. The method of claim 50, wherein the cells are mouse cells.

54. A method of purifying CFU-E cells from a population of cells that comprises one or more CFU-E cells, the method comprising steps of: (a) depleting the population of cells that are positive for Ter119, CD16, CD32, Sca-1, and/or CD41; and (b) selecting cells that are (i) c-kit positive and (ii) CD71^{20% high}.

55. The method of claim 54, wherein the cells are selected from the group consisting of bone marrow cells, umbilical cord blood cells, peripheral blood cells, and fetal liver cells.

56. The method of claim 54, wherein the cells are mouse cells.

57. A method for determining whether a compound promotes expansion of BFU-E cells comprising (a) providing a purified cell population comprising at least 80% BFU-E cells; (b) contacting the purified cell population of (a) with a test compound; (c) assessing the extent to which the cell population increases during a subsequent culture period, wherein if the cell population increases to a greater extent than would be expected had the cell population not been contacted with the compound, then the compound promotes expansion of BFU-E cells.

58. The method of claim 57, wherein the method comprises assessing the number of BFU-E-derived colonies cells that develop from at least some of the cells contacted with the compound.

59. The method of claim 57, wherein the compound is a GR agonist.

60. The method of claim 57, wherein the method comprises screening a compound collection comprising at least 10 GR agonists.

61. A cell culture medium comprising a HIF-1a activator and a GR agonist.

62. The cell culture medium of claim 61, wherein the medium comprises Epo.

63. The cell culture medium of claim 61, wherein the medium comprises Epo and SCF.

64. The cell culture medium of claim 61, wherein the medium comprises Epo, SCF, and IGF-1.

65. The cell culture medium of claim 61, wherein the HIF-1a activator is a PHI.

66. The cell culture medium of claim 61, wherein the HIF-1a activator is DMOG.

67. The cell culture medium of claim 61, wherein the GR agonist is a glucocorticoid.

68. The cell culture medium of claim 61, wherein the GR agonist is dexamethasone.

69. The cell culture medium of claim 61, wherein the HIF-1a activator is a prolyl hydroxylase inhibitor and the GR agonist is a glucocorticoid.

70. The cell culture medium of claim 53, wherein the medium is serum-free.

71. A container containing the cell culture medium of claim 61, wherein said container is suitable for expanding cells.

72. The container of claim 71, wherein the container further contains erythroid progenitor cells.

73. The container of claim 71, wherein the container further contains human erythroid progenitor cells.

74. The cell culture medium of claim 61, wherein the medium is serum-free.

75. A pharmaceutical composition comprising a HIF-1a activator and a GR agonist.

76. The pharmaceutical composition of claim 75, wherein the HIF-1a activator is a PHI.

77. The pharmaceutical composition of claim 75, wherein the GR agonist is a glucocorticoid.

78. The pharmaceutical composition of claim 75, wherein the HIF-1a activator is a PHI and the GR agonist is a glucocorticoid.

79. The pharmaceutical composition of claim 75, wherein the pharmaceutical composition is suitable for oral administration.
